(19) United States
(12) Patent Application Publication
(10) Pub. No.: US 2004/0214868 A1
(43) Pub. Date: Oct. 28, 2004

(54) Amino nicotinate derivatives as glucokinase (GLK) modulators

(76) Inventors: Barry Raymond Hayter, Cheshire (GB); Gordon Stuart Currie, Cheshire (GB); Rodney Brian Hargreaves, Cheshire (GB); Roger James, Cheshire (GB); Clifford David Jones, Cheshire (GB); Darren McKerrecher, Cheshire (GB); Joanne Victoria Allen, Cheshire (GB); Peter William Rodney Caulkett, Cheshire (GB); Craig Johnstone, Cheshire (GB); Harold Gaskin, Cheshire (GB)

Correspondence Address: ROPES & GRAY LLP, ONE INTERNATIONAL PLACE, BOSTON, MA, 02110-2624, US

(21) Appl. No.: 10/482,572

(22) PCT Filed: Jun. 24, 2002
(86) PCT No.: PCT/GB02/02873

(30) Foreign Application Priority Data
Jun. 26, 2001 (SE) 0102300-1

(51) Int. Cl. C07D 213/72; A61K 31/44

(52) U.S. Cl. 514/352; 546/308

(57) ABSTRACT

The invention related to novel compounds of Formula (I) or a salt, solvate or prodrug thereof, wherein R¹, R², R³, n and m are as described in the specification, useful in the treatment of a

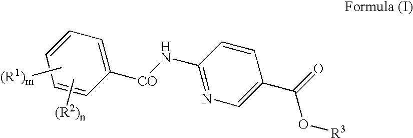

disease or condition mediated through glucokinase (GLK), such as type 2 diabetes. The invention also relates to methods for preparing compounds of Formula (I) and their use as medicaments in the treatment of diseases mediated by glucokinase.

### CROSS-REFERENCE TO RELATED APPLICATIONS

This application is a national stage filing under 35 U.S.C. 371 of International Application No. PCT/GBO2/02873, filed Jun. 24, 2002, which claims priority from United Kingdom Patent Application No. 0102300-1, filed Jun. 26, 2001, the specifications of which are incorporated by reference herein. International Application No. PCT/GB02/02873 was published under PCT Article 21(2) in English.

### FIELD OF THE INVENTION

The present invention relates to compounds which activate glucokinase (GLK), leading to a decreased glucose threshold for insulin secretion. In addition the compounds are predicted to lower blood glucose by increasing hepatic glucose uptake. Such compounds may have utility in the treatment of Type 2 diabetes and obesity. The invention also relates to pharmaceutical compositions comprising a compound of the invention, and use of such a compound in the conditions described above.

In the pancreatic β-cell and liver parenchymal cells the main plasma membrane glucose transporter is GLUT2. Under physiological glucose concentrations the rate at which GLUT2 transports glucose across the membrane is not rate limiting to the overall rate of glucose uptake in these cells. The rate of glucose uptake is limited by the rate of phosphorylation of glucose to glucose-6-phosphate (G-6-P) which is catalysed by glucokinase (GLK) [1]. GLK has a high (6-10 mM) Km for glucose and is not inhibited by physiological concentrations of G-6-P [1]. GLK expression is limited to a few tissues and cell types, most notably pancreatic β-cells and liver cells (hepatocytes) [1]. In these cells GLK activity is rate limiting for glucose utilisation and therefore regulates the extent of glucose induced insulin secretion and hepatic glycogen synthesis. These processes are critical in the maintenance of whole body glucose homeostasis and both are dysfunctional in diabetes [2].

In one sub-type of diabetes, Type 2 maturity-onset diabetes of the young (MODY-2), the diabetes is caused by GLK loss of function mutations [3, 4]. Hyperglycaemia in MODY-2 patients results from defective glucose utilisation in both the pancreas and liver [5]. Defective glucose utilisation in the pancreas of MODY-2 patients results in a raised threshold for glucose stimulated insulin secretion. Conversely, rare activating mutations of GLK reduce this threshold resulting in familial hyperinsulinism [6, 7]. In addition to the reduced GLK activity observed in MODY-2 diabetics, hepatic glucokinase activity is also decreased in type 2 diabetics [8]. Importantly, global or liver selective overexpression of GLK prevents or reverses the development of the diabetic phenotype in both dietary and genetic models of the disease [9-12]. Moreover, acute treatment of type diabetics with fructose improves glucose tolerance through stimulation of hepatic glucose utilisation [13]. This effect is believed to be mediated through a fructose induced increase in cytosolic GLK activity in the hepatocyte by the mechanism described below [13].

Hepatic GLK activity is inhibited through association with GLK regulatory protein (GLKRP). The GLK/GLKRP complex is stabilised by fructose-6-phosphate (F6P) binding to the GLKRP and destabilised by displacement of this sugar phosphate by fructose-1-phosphate (F1P). F1P is generated by fructokinase mediated phosphorylation of dietary fructose. Consequently, GLK/GLKRP complex integrity and hepatic GLK activity is regulated in a nutritionally dependent manner as F6P is elevated in the post-absorptive state whereas F1P predominates in the post-prandial state. In contrast to the hepatocyte, the pancreatic 1-cell expresses GLK in the absence of GLKRP. Therefore, β-cell GLK activity is regulated exclusively by the availability of its substrate, glucose. Small molecules may activate GLK either directly or through destabilising the GLK/GLKRP complex. The former class of compounds are predicted to stimulate glucose utilisation in both the liver and the pancreas whereas the latter are predicted to act exclusively in the liver. However, compounds with either profile are predicted to be of therapeutic benefit in treating Type 2 diabetes as this disease is characterised by defective glucose utilisation in both tissues.

GLK and GLKRP and the K_{ATP} channel are expressed in neurones of the hypothalamus, a region of the brain that is important in the regulation of energy balance and the control of food intake [14-18]. These neurones have been shown to express orectic and anorectic neuropeptides [15, 19, 20] and have been assumed to be the glucose-sensing neurones within the hypothalamus that are either inhibited or excited by changes in ambient glucose concentrations [17, 19, 21, 22]. The ability of these neurones to sense changes in glucose levels is defective in a variety of genetic and experimentally induced models of obesity [23-281. Intracerebroventricular (icv) infusion of glucose analogues, that are competitive inhibitors of glucokinase, stimulate food intake in lean rats [29, 30]. In contrast, icv infusion of glucose suppresses feeding [31]. Thus, small molecule activators of GLK may decrease food intake and weight gain through central effects on GLK. Therefore, GLK activators may be of therapeutic use in treating eating disorders, including obesity, in addition to diabetes. The hypothalamic effects will be additive or synergistic to the effects of the same compounds acting in the liver and/or pancreas in normalising glucose homeostasis, for the treatment of Type 2 diabetes. Thus the GLK/GLKRP system can be described as a potential “Diabesity” target (of benefit in both Diabetes and Obesity).

In WO0058293 and WO 01/44216 (Roche), a series of benzylcarbamoyl compounds are described as glucokinase activators. The mechanism by which such compounds activate GLK is assessed by measuring the direct effect of such compounds in an assay in which GLK activity is linked to NADH production, which in turn is measured optically—see details of the in vitro assay described in Example A.

In WO9622282/93/94/95 and WO9749707/8 are disclosed a number of intermediates used in the preparation of compounds useful as vasopressin agents which are related to those disclosed in the present invention. Related compounds are also disclosed in WO9641795 and JP8143565 (vasopressin antagonism), in JP8301760 (skin damage prevention) and in EP619116 (osetopathy).

We present as a feature of the invention the use of a compound of Formula (I) or a salt, pro-drug or solvate thereof, in the preparation of a medicament for use in the treatment or prevention of a disease or medical condition mediated through GLK:

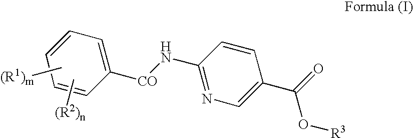

wherein

m is 0, 1 or 2;

n is 0, 1, 2, 3 or 4;

and n+m>0;

each R¹ is independently selected from OH, —(CH₂)₁₋₄OH, —CH₃₋ₐFₐ, —(CH₂)₁₋₄CH₃₋ₐFₐ, halo, C₁₋₆alkyl, C₂₋₆alkenyl, C₂₋₆alkynyl, NO₂, NH₂, —NH—C₁₋₄alkyl, —N-di-(C₁₋₄alkyl), CN or formyl;

each R² is the group Y—X—

wherein each X is a linker independently selected from:

—O—Z—, —O—Z—O—Z—, —C(O)O—Z—, —OC(O)—Z—, —S—Z—, —SO—Z—, —SO₂—Z—, —N(R⁶)—Z—, —N(R⁶)SO₂—Z—, —SO₂N(R⁶)—Z—, —(CH₂)₁₋₄—, —CH═CH—Z—, —C≡C—Z—, —N(R⁶)CO—Z—, —CON(R⁶)—Z—, —C(O)N(R⁶)S(O)₂—Z—, —S(O)₂N(R⁶)C(O)—Z—, —C(O)—Z— or a direct bond;

each Z is independently a direct bond or a group of the formula

—(CH₂)ₚ—C(R⁶)₂—(CH₂)_{q}—;

each Y is independently selected from aryl-Z¹-, heterocyclyl-Z¹-, C₃₋₇cycloalkyl-Z¹—, C₁₋₆alkyl, C₂₋₆alkenyl, C₂₋₆alkynyl or —(CH₂)₁₋₄CH₃₋ₐFₐ;

wherein each Y is independently optionally substituted by up to 3 R⁴ groups;

each R⁴ is independently selected from halo, —CH₃₋ₐFₐ, CN, NO₂, NH₂, C₁₋₆alkyl, —OC₁₋₆alkyl, —COOH, —C(O)OC₁₋₆alkyl, OH or phenyl, or R⁵—X¹—, where X¹ is independently as defined in X above and R⁵ is selected from hydrogen, C₁₋₆alkyl, —CH₃₋ₐFₐ, phenyl, naphthyl, heterocyclyl or C₃₋₇cycloalkyl; and R⁵ is optionally substituted by halo, C₁₋₆alkyl, —CH₃₋ₐFₐ, CN, NO₂, NH₂, COOH or —C(O)OC₁₋₆alkyl, wherein each phenyl, naphthyl or heterocyclyl ring in R⁵ is optionally substituted by halo, CH₃₋ₐFₐ, CN, NO₂, NH₂, C₁₋₆alkyl, —OC₁₋₆alkyl, COOH, —C(O)OC₁₋₆alkyl or OH;

each Z¹ is independently a direct bond or a group of the formula

—(CH₂)ₚ—C(R⁶)₂—(CH₂)_{q}—;

R³ is selected from hydrogen or C₁₋₆alkyl; and

R⁶ is independently selected from hydrogen, C₁₋₆alkyl or —C₂₋₄alkyl-O—C₁₋₄alkyl;

each a is independently 1, 2 or 3;

p is an integer between 0 and 2;

q is an integer between 0 and 2;

and p+q<4.

According to a further feature of the invention there is provided the use of a compound of Formula (Ia) or a salt, pro-drug or solvate thereof, in the preparation of a medicament for use in the treatment or prevention of a disease or medical condition mediated through GLK:

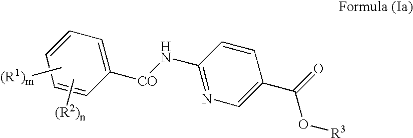

wherein

m is 0, 1 or 2;

n is 0, 1, 2, 3 or 4;

and n+m>0;

each R¹ is independently selected from OH, —(CH₂)₁₋₄OH, —CH₃₋ₐFₐ, —(CH₂)₁₋₄CH₃₋ₐFₐ, halo, C₂₋₆alkenyl, C₂₋₆alkynyl, NO₂, NH2, or CN;

each R² is the group Y—X—

wherein each X is a linker independently selected from:

—O(CH₂)₀₋₂—, —(CH₂)₀₋₂O—, —C(O)O(CH₂)₀₋₂—, —S(CH₂)₀₋₂—, —SO(CH₂)₀₋₂—, —SO₂(CH₂)₀₋₂-,

—NHSO₂, —SO₂NH—, —(CH₂)₁₄—, —CH═CH(CH₂)₀₋₂—, —C≡C(CH₂)₀₋₂—, —NHCO—, or —CONH—;

each Y is independently selected from phenyl(CH₂)₀₋₂, naphthyl(CH₂)0₂, heterocyclyl(CH₂)₀₋₂, C₃₋₇ cycloalkyl(CH₂)₀₋₂, C₁₋₆ alkyl, C₂₋₆ alkenyl or C₂₋₆ alkynyl; and each Y is independently optionally substituted by R⁴;

each R⁴ is independently selected from halo, —CH₃₋ₐFₐ, CN, NO₂, NH₂, C₁₋₆alkyl, —OC₁₋₆alkyl, COOH, —C(O)OC₁₋₆alkyl, OH, phenyl,

or R⁵—X¹—, where X¹ is independently as defined for X above, and R⁵ is selected from hydrogen, C₁₋₆alkyl, —CH₃₋ₐFₐ, phenyl, naphthyl, heterocyclyl or C₃₋₇cycloalkyl;

and R⁵ is optionally substituted by halo, C₁₋₆alkyl, —CH₃₋ₐFₐ, CN, NO₂, NH₂, COOH and —C(O)OC₁₋₆alkyl;

each a is independently 1, 2 or 3;

R³ is selected from hydrogen or C₁₋₆alkyl.

According to a further feature of the invention there is provide a compound of Formula (Ib) or a salt, solvate or pro-drug thereof;

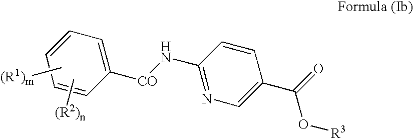

wherein

m is 0, 1 or 2;

n is 0, 1, 2, 3 or 4; and n+m>0;

each R¹ is independently selected from OH, —(CH₂)₁₋₄OH, —CH₃₋ₐFₐ, —(CH₂)₁₋₄CH₃₋ₐFₐ, halo, C₁₋₆alkyl, C₂₋₆alkenyl, C₂₋₆alkynyl, NO₂, Nf2, —NH—C₁₋₄alkyl, —N-di-(C₁₋₄alkyl), CN or formyl;

each R² is the group Y—X—

wherein each X is a linker independently selected from:

—O-Z-, —O-Z-O-Z-, —C(O)O-Z-, —OC(O)-Z-, —S-Z-, —SO-Z-, —SO₂-Z-, —N(R⁶)-Z-, —N(R⁶)SO₂-Z-, —SO₂N(R⁶)-Z-, —(CH₂)₁₋₄—, —CH═CH-Z-, —C≡C-Z-, —N(R⁶)CO-Z-, —CON(R⁶)-Z-, —C(O)N(R⁶)S(O)₂-Z-, —S(O)₂N(R⁶)C(O)-Z-, —C(O)-Z- or a direct bond;

each Z is independently a direct bond or a group of the formula

—(CH₂)ₚ—C(R⁶)₂—(CH₂)_{q}—;

each Y is independently selected from aryl-Z¹-, heterocyclyl-Z¹-, C₃₋₇cycloalkyl-Z¹-, C₁₋₆alkyl, C₂₋₆alkenyl, C₂₋₆alkynyl or —(CH₂)₁₋₄CH₃₋ₐFₐ;

wherein each Y is independently optionally substituted by up to 3 R⁴ groups;

each R⁴ is independently selected from halo, —CH₃₋ₐFₐ, CN, NO₂, NH₂, C₁₋₆alkyl, —OC₁₋₆alkyl, —COOH, —C(O)OC₁₋₆alkyl, OH or phenyl, or R⁵—X¹—, where X¹ is independently as defined in X above and R⁵ is selected from hydrogen, C₁₋₆alkyl, —CH₃₋ₐFₐ, phenyl, naphthyl, heterocyclyl or C₃₋₇cycloalkyl; and R⁵ is optionally substituted by halo, C₁₋₆alkyl, —CH₃₋ₐFₐ, CN, NO₂, NH₂, COOH or —C(O)OC₁₋₆alkyl, wherein each phenyl, naphthyl or heterocyclyl ring in R⁵ is optionally substituted by halo, CH₃₋ₐFₐ, CN, NO₂, NH₂, C₁₋₆alkyl, —OC₁₋₆alkyl, COOH, —C(O)OC₁₋₆alkyl or OH;

each Z¹ is independently a direct bond or a group of the formula

—(CH₂)ₚ—C(R⁶)₂—(CH₂)_{q}—;

R³ is selected from hydrogen or C₁₋₆alkyl; and

R⁶ is independently selected from hydrogen, C₁₋₆alkyl or —C₂₋₄alkyl-O—C₁₋₄alkyl;

each a is independently 1, 2 or 3;

p is an integer between 0 and 2;

q is an integer between 0 and 2;

and p+q<4.

with the proviso that:

(i) when R³ is hydrogen or methyl, m is 1 and n is 0 then R¹ cannot be 2-halo or 2-methyl;

(ii) when R³ is hydrogen or methyl, m is 2 and n is 0 then (R¹)ₘ is other than di-C₁₋₄alkyl, di-halo or mono-halo-mono-C₁₋₄alkyl;

(iii) when R³ is hydrogen, methyl or ethyl, m is 0, n is 1, R² is a substituent at the −2 position or 4-position and X is —O— or a direct bond then Y cannot be methyl, phenyl or benzyl and R⁴ (when present) cannot be methyl or trifluoromethyl;

(iv) when R³ is hydrogen, m is 0, n is 2, X is a direct bond then (R²)ₘ is other than 2,4-diphenyl;

(v) when R³ is hydrogen, m is 0 and n is 3 then at least one R² must be other than methoxy (preferably at least two of the R² groups must be other than methoxy, most preferably each R² must be other than methoxy); and

(vi) the following compound is excluded:

ethyl 6-[(3-tert-butyl-2-hydroxy-6-methyl-5-nitrobenzoyl)amino]nicotinate.

According to a further feature of the invention there is provided a compound of Formula (Ic) or a salt, solvate or pro-drug thereof;

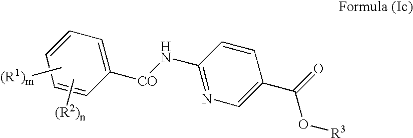

wherein

m is 0, 1 or 2;

n is 0, 1, 2, 3 or 4;

and n+m>0;

each R¹ is independently selected from OH, —(CH₂)₁₋₄OH, —CH₃₋ₐFₐ, —(CH₂)₁₋₄CH₃₋ₐFₐ, halo, C₂₋₆alkenyl, C₂₋₆alkynyl, NO₂, NH₂, or CN;

each R² is the group Y—X—

wherein each X is a linker independently selected from:

—O(CH₂)₀₋₂—, —(CH₂)₀₋₂O—, —C(O)O(CH₂)₀₋₂—, —S(CH₂)₀₋₂—, —SO(CH₂)₀₋₂—, —SO₂(CH₂)_{0-2—,}

—NHSO₂, —SO₂NH—, —(CH₂)₁₋₄—, —CH═CH(CH₂)₀₋₂—, —C≡C(CH₂)₀₋₂—, —NHCO—, or —CONH—;

each Y is independently selected from phenyl(CH₂)₀₋₂, naphthyl(CH₂)₀₋₂, heterocyclyl(CH₂)₀₋₂, C₃₋₇ cycloalkyl(CH₂)₀₋₂, C₁₋₆alkyl, C₂₋₆alkenyl or C₂₋₆alkynyl; and each Y is independently optionally substituted by R⁴;

each R⁴ is independently selected from halo, CH₃₋ₐFₐ, CN, NO₂, NH₂, C₁₋₆alkyl, OC₁₋₆alkyl, COOH, C(O)OC₁₋₆alkyl, OH, phenyl,

or R⁵—X¹—, where X is independently as defined for X above, and R⁵ is selected from hydrogen, C₁₋₆alkyl, CH₃₋ₐFₐ, phenyl, naphthyl, heterocyclyl or C₃₋₇cycloalkyl;

and R⁵ is optionally substituted by halo, C₁₋₆alkyl, —CH₃₋ₐFₐ, CN, NO₂, NH₂, COOH and —C(O)OC₁₋₆alkyl;

each a is independently 1, 2 or 3;

R³ is selected from hydrogen or C₁₋₆alkyl.

with the proviso that:

(i) when R³ is hydrogen or methyl, m is 1 and n is 0 then R¹ cannot be halo or methyl;

(ii) when R³ is hydrogen or methyl, m is 2 and n is 0 then (R¹)ₘ is other than di-C₁₋₄alkyl, di-halo or mono-halo-mono-C₁₋₄alkyl;

(iii) when R³ is hydrogen or methyl, m is 0, n is 1, R² is a substituent at the −2 position and X is—O— then Y cannot be methyl or benzyl; and

(iv) provided that when R³ is hydrogen, m is 0 and n is 3 then at least one R² must be other than methoxy (preferably at least two of the R² groups must be other than methoxy, most preferably each R² must be other than methoxy).

Compounds of the invention may form salts which are within the ambit of the invention. Pharmaceutically acceptable salts are preferred although other salts may be useful in, for example, isolating or purifying compounds.

The term “aryl” refers to phenyl, naphthyl or a partially saturated bicyclic carbocyclic ring containing between 8 and 12 carbon atoms, preferably between 8 and 10 carbon atoms. Example of partially saturated bicyclic carbocyclic ring include: 1,2,3,4-tetrahydronaphthyl, indanyl, indenyl, 1,2,4a,5,8,8a-hexahydronaphthyyl or 1,3a-dihydropentalene.

The term “halo” includes fluoro, chloro, bromo and iodo; preferably chloro, bromo and fluoro; most preferably fluoro.

The expression “—CH₃₋ₐFₐ” wherein a is an integer between 1 and 3 refers to a methyl group in which 1, 2 or all 3 hydrogen are replaced by a fluorine atom.

Examples include: trifluoromethyl, difluoromethyl and fluoromethyl An analogous notation is used with reference to the group —(CH₂)₁₋₄CH₃₋ₐFₐ, examples include: 2,2-difluoroethyl and 3,3,3-trifluoropropyl.

In this specification the term “alkyl” includes both straight and branched chain alkyl groups. For example, “C₁₋₄alkyl” includes propyl, isopropyl and t-butyl.

The term “heterocyclyl” is a saturated, partially saturated or unsaturated, mono or bicyclic ring containing 3-12 atoms of which at least one atom is chosen from nitrogen, sulphur or oxygen, which may, unless otherwise specified, be carbon or nitrogen linked, wherein a —CH₂— group can optionally be replaced by a —C(O)— and sulphur atoms in a heterocyclic ring may be oxidised to S(O) or S(O)₂ groups. Preferably a “heterocyclyl” is a saturated, partially saturated or unsaturated, mono or bicyclic ring (preferably monocyclic of 5 or 6 atoms) containing 9 or 10 atoms of which 1 to 3 atoms are nitrogen, sulphur or oxygen, which may, unless otherwise specified, be carbon or nitrogen linked, wherein a —CH₂— group can optionally be replaced by a —C(O)— or sulphur atoms in a heterocyclic ring may be oxidised to S(O) or S(O)₂ groups. Examples and suitable values of the term “heterocyclyl” are thiazolidinyl, pyrrolidinyl, pyrrolinyl, 2,5-dioxopyrrolidinyl, 2-benzoxazolinonyl, 1,1-dioxotetrahydrothienyl, 2,4-dioxoimidazolidinyl, 2-oxo-1,3,4-(4-triazolinyl), 2-oxazolidinonyl, 5,6-dihydrouracilyl, 1,3-benzodioxolyl, 1,2,4-oxadiazolyl, 2-azabicyclo[2.2.1]heptyl, 4-thiazolidonyl, morpholino, furanyl, 2-oxotetrahydrofuranyl, tetrahydrofuranyl, 2,3-dihydrobenzofuranyl, benzothienyl, isoxazolyl, tetrahydropyranyl, piperidyl, 1-oxo-1,3-dihydroisoindolyl, piperazinyl, thiomorpholino, 1,1-dioxothiomorpholino, tetrahydropyranyl, 1,3-dioxolanyl, homopiperazinyl, thienyl, isoxazolyl, imidazolyl, pyrrolyl, thiazolyl, thiadiazolyl, isothiazolyl, 1,2,4-triazolyl, 1,2,3-triazolyl, pyranyl, indolyl, pyrimidyl, pyrazinyl, pyridazinyl, pyridyl, 4-pyridonyl, quinolyl, tetrahydrothienyl 1,1-dioxide, 2-oxo-pyrrolidinyl and 1-isoquinolonyl. Preferred examples of “heterocyclyl” when referring to a ⅚ and 6/6 bicyclic ring system include chromanyl, benzofuranyl, benzimidazolyl, benzthiophenyl, benzthiazolyl, benzisothiazolyl, benzoxazolyl, benzisoxazolyl, pyridoimidazolyl, pyrimidoimidazolyl, quinolinyl, isoquinolinyl, quinoxalinyl, quinazolinyl, phthalazinyl, cinnolinyl, imidazo[2,1-b][1,3]thiazolyl and naphthyridinyl. Preferably the term “heterocyclyl” refers to 5- or 6-membered monocyclic heterocyclic rings, such as oxazolyl, isoxazolyl, pyrrolidinyl, 2-pyrrolidonyl, 2,5-dioxopyrrolidinyl, morpholino, furanyl, tetrahydrofliranyl, piperidyl, piperazinyl, thiomorpholino, tetrahydropyranyl, homopiperazinyl, thienyl, imidazolyl, 1,2,4-triazolyl, 1,3,4-triazolyl, indolyl, thiazolyl, thiadiazolyl, pyrazinyl, pyridazinyl and pyridyl.

The term “cycloalkyl” refers to a saturated carbocylic ring containing between 3 to 12 carbon atoms, preferably between 3 and 7 carbon atoms. Examples of C₃₋₇cycloalkyl include cycloheptyl, cyclohexyl, cyclopentyl, cyclobutyl or cyclopropyl. Preferably cyclopropyl, cyclopentyl or cyclohexyl.

Examples of C₁₋₆alkyl include methyl, ethyl, propyl, isopropyl, 1-methyl-propyl, sec-butyl, tert-butyl and 2-ethyl-butyl; examples of C₂₋₆alkenyl include: ethenyl, 2-propenyl, 2-butenyl, or 2-methyl-2-butenyl; examples of C₂₋₆alkynyl include: ethynyl, 2-propynyl, 2-butynyl, or 2-methyl-2-butynyl, examples of —OC₁₋₄alkyl include methoxy, ethoxy, propoxy and tert-butoxy; examples of —C(O)OC₁₋₆alkyl include methoxycarbonyl, ethoxycarbonyl and tert-butyloxycarbonyl; examples of —NH—C₁₋₄alkyl include:

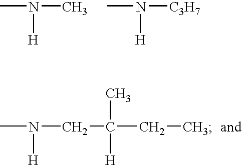

examples of —N-di-(C₁₋₄alkyl):

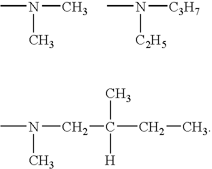

For the avoidance of doubt, in the definition of linker group ‘X’, the right hand side of the group is attached to the phenyl ring and the left hand side is bound to ‘Y’.

It is to be understood that, insofar as certain of the compounds of the invention may exist in optically active or racemic forms by virtue of one or more asymmetric carbon atoms, the invention includes in its definition any such optically active or racemic form which possesses the property of stimulating GLK directly or inhibiting the GLK/GLKRP interaction. The synthesis of optically active forms may be carried out by standard techniques of organic chemistry well known in the art, for example by synthesis from optically active starting materials or by resolution of a racemic form.

Preferred compounds of Formula (I) to (Ic) above or of Formula (II) to (IIf) below are those wherein any one or more of the following apply:

(1) m is 0 or 1;

n is 1 or 2; preferably n is 2;

most preferably m is 0 and n is 2.

(2) The R¹ and/or R²group(s) are attached at the 2-, 3- or 5-position relative to the carbonyl group; when n+m is 3, the groups are preferably at the 2-, 3- and 5-positions; when n+m is 2, the groups are preferably at the 3- and 5-positions; most preferably there are two groups in total, substituted at the 3- and 5-positions.

(3) each R¹ is independently selected from OH, CH₃₋ₐFₐ (preferably CF₃), halo, C₁₋₄alkyl (preferably methyl) and CN; preferably R¹ is selected from CH₃₋ₐFₐ (preferably CF₃), halo, C₁₋₄ alkyl (preferably methyl) and CN; most preferably R¹ is selected from —CH₃₋ₐFₐ (preferably —CF₃), or halo.

(4) each R² is the group Y—X—

wherein each X is independently selected from:

—O-Z-, —C(O)O-Z-, —S-Z-, —SO-Z-, —SO₂-Z-, —N(R⁶)CO-Z-, —CON(R⁶)-Z-, —SO₂N(R⁶)-Z-, —N(R⁶)SO₂-Z- or —CH═CH-Z-;

preferably each X is selected from:

—O-Z-, —S-Z-, —SO-Z-, —SO₂-Z-, —CON(R⁶)-Z-, —SO₂N(R⁶)-Z-, or —CH═CH-Z-;

further preferably each X is selected from:

—O-Z-, —N(R⁶)-Z-, —CH═CH-Z-, —SO₂N(R⁶)-Z- or —S-Z-;

Most preferably each X is selected from:

—O-Z-, —SO₂N(R⁶)-Z- or —N(R⁶)-Z-.

each Z is independently selected from:

a direct bond or —(CH₂)₁₋₂, or a group of the formula —(CH₂)ₚ—C(R⁶)₂—(CH₂)_{q}—, wherein one R⁶ group is hydrogen and the other R⁶ group is C₁₋₄alkyl;

preferably a direct bond, —(CH₂)₀₋₂— or

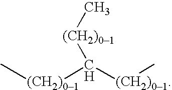

more preferably a direct bond or —CH₂—.

each Z¹ is independently selected from:

a direct bond or —(CH₂)₁₋₂, or a group of the formula —(CH₂)ₚ—C(R⁶)₂—(CH₂)_{q}—, wherein one R⁶ group is hydrogen and the other R⁶ group is C₁₋₄alkyl;

preferably a direct bond, —(CH₂)₀₋₂— or

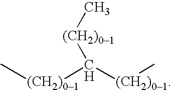

more preferably a direct bond, —CH₂—, —(CH₂)₂— or

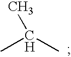

most preferably —CH₂— or a direct bond.

and each Y is independently selected from:

aryl-Z¹-, heterocyclyl-Z¹-, or C₃₋₇cycloalkyl-Z¹-,

C₁₋₆ alkyl or C₂₋₆ alkenyl;

preferably each Y is selected from:

phenyl-Z¹-, naphthyl-Z¹-, heterocyclyl-Z¹-, or C₁₋₆ alkyl (preferably a branched chain C₂₋₆ alkyl such as isopropyl or isobutyl);

wherein each Y is independently optionally substituted by R⁴.

(5) each R² is the group Y—X—, Z within the definition of X is a direct bond and Z¹ within the definition of Y is a group of the formula (CH₂)ₚ—C(R⁶)₂—(CH₂)_{q}—.

(6) each R⁴ is independently selected from:

halo, —CH₃₋ₐFₐ CN, NO₂, C₁₋₆alkyl, OC₁₋₆alkyl, —COOH, —C(O)OC₁₋₆alkyl, OH, heterocyclyl or phenyl;

preferably each R⁴ is selected from:

halo, —CH₃₋ₐFₐ, CN, C₁₋₆alkyl (preferably methyl), —COOH or phenyl.

Most preferably R⁴ is selected from: F, Cl, methyl or CN.

(7) R³ is selected from hydrogen or C₁₋₆alkyl; preferably R³ is selected from hydrogen or methyl; most preferably R³ is hydrogen.

According to a further feature of the invention there is provided the following preferred groups of compounds of the invention:

(I) a compound of Formula (II)

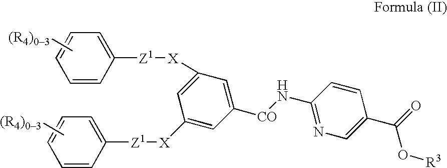

wherein:

X, Z¹, R³ and R⁴ are as defined above in a compound of Formula (I);

or a salt, solvate or pro-drug thereof.

(II) a compound of Formula (IIa)

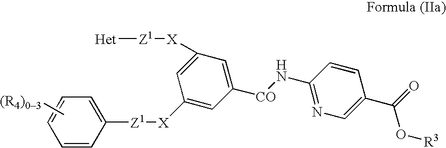

wherein:

Het is a monocyclic heterocyclyl, optionally substituted with up to 3 groups selected from R⁴ and,

X, Z¹, R³ and R⁴ are as defined above in a compound of Formula (I);

or a salt, solvate or pro-drug thereof.

(III) a compound of Formula (IIb)

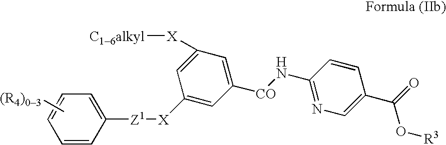

wherein:

the C₁₋₆alkyl group is optionally substituted with up to 3 groups selected from R⁴, preferably unsubstituted;

the C₁₋₆alkyl group optionally contains a double bond, preferably the C₁₋₆alkyl group does not contains a double bond; and

X, Z¹, R³ and R⁴ are as defined above in a compound of Formula (I);

or a salt, solvate or pro-drug thereof.

(IV) a compound of Formula (IIc)

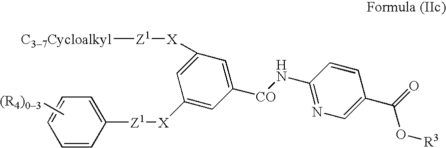

wherein:

the C₃₋₇cycloalkyl group is optionally substituted with up to 3 groups selected from R⁴, and

X, Z¹, R³ and R⁴ are as defined above in a compound of Formula (I);

or a salt, solvate or pro-drug thereof.

(V) a compound of Formula (IId)

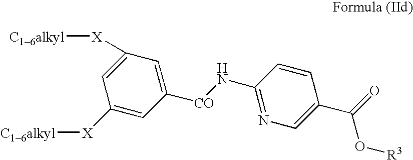

wherein:

the C₁₋₆alkyl groups are independently optionally substituted with up to 3 groups selected from R⁴, preferably one of the C₁₋₆alkyl groups is unsubstituted,

the C₁₋₆alkyl groups independently optionally contain a double bond, preferably only one of the C₁₋₆alkyl groups contain a double bond, preferably neither of the C₁₋₆alkyl group contains a double bond, and

X, R³ and R⁴ are as defined above in a compound of Formula (I);

or a salt, solvate or pro-drug thereof.

(VI) a compound of Formula (IIe)

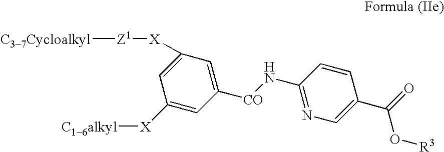

wherein:

the C₃₋₇cycloalkyl and C₁₋₆alkyl groups are independently optionally substituted with up to 3 groups selected from R⁴, preferably the C₁₋₆alkyl group is unsubstituted;

the C₁₋₆alkyl group optionally contains a double bond, preferably the C₁₋₆alkyl group does not contains a double bond; and

X, Z¹, R³ and R⁴ are as defined above in a compound of Formula (I);

or a salt, solvate or pro-drug thereof.

(VII) a compound of Formula (IIf)

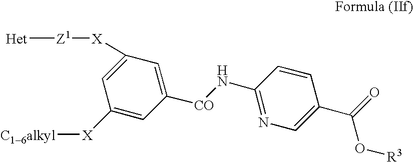

wherein:

Het is a monocyclic heterocyclyl,

the Het and C₁₋₆alkyl groups are independently optionally substituted with up to 3 groups selected from R⁴, preferably the C₁₋₆alkyl group is unsubstituted;

the C₁₋₆alkyl group optionally contains a double bond, preferably the C₁₋₆alkyl group does not contains a double bond; and

X, Z¹, R³ and R⁴ are as defined above in a compound of Formula (I);

or a salt, solvate or pro-drug thereof.

(VIII) a compound of Formula (IIg)

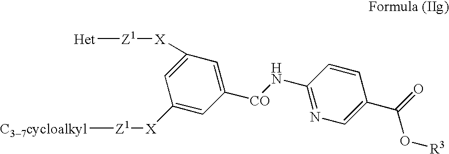

wherein:

Het is a monocyclic heterocyclyl,

the Het and C₃₋₇cycloalkyl groups are independently optionally substituted with up to 3 groups selected from R⁴, and

X, Z¹, R³ and R⁴ are as defined above in a compound of Formula (I);

or a salt, solvate or pro-drug thereof.

(IX) a compound of Formula (IIh)

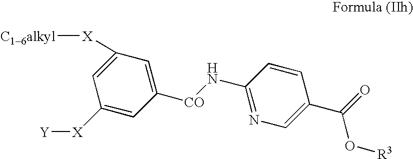

wherein:

Y is aryl-Z¹-, wherein aryl is preferably a partially saturated bicyclic carbocyclic ring;

Y and the C₁₋₆alkyl group are independently optionally substituted with up to 3 groups selected from R⁴, preferably the C₁₋₆alkyl group is unsubstituted,

the C₁₋₆alkyl group optionally contains a double bond, preferably the C₁₋₆alkyl group does not contains a double bond; and

X, Z¹, R³ and R⁴ are as defined above in a compound of Formula (I);

or a salt, solvate or pro-drug thereof.

(X) a compound of Formula (IIj)

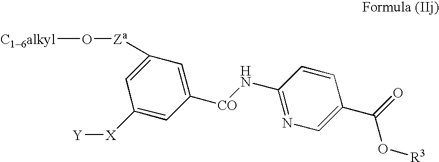

wherein:

X is selected from —SO₂N(R⁶)-Z- or —N(R⁶)SO₂-Z-, preferably X is —SO₂N(R⁶)-Z-;

Z is as described above, preferably Z is propylene, ethylene or methylene, more preferably Z is methylene;

Z^{a} is selected from a direct bond or a group of the formula —(CH₂)ₚ—C(R⁶)₂—(CH₂)_{q}—; preferably Z^{a} is selected from C₁₋₂alkylene or a direct bond; preferably Z^{a} is a direct bond;

R⁶ is selected from: C₁₋₄alkyl or hydrogen, preferably methyl or hydrogen;

Y is selected from aryl-Z¹- or heterocyclyl-Z¹-;

Y and the C₁₋₆alkyl group are independently optionally substituted with up to 3 groups selected from R⁴,

the C₁₋₆alkyl group optionally contains a double bond, preferably the C₁₋₆alkyl group does not contain a double bond, and

Z¹, R³ and R⁴ are as defined above in a compound of Formula (I);

or a salt, solvate or pro-drug thereof.

A further preferred groups of compounds of the invention in either of groups (I)-(IX) above is wherein:

X is independently selected from: —O-Z-, SO₂N(R⁶)-Z- or —N(R⁶)-Z-;

Z is a direct bond or —CH₂—;

Z¹ is selected from a direct bond, —CH₂——(CH₂)₂— or

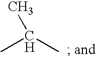

R³ is as defined above in a compound of Formula (I);

or a salt, solvate or pro-drug thereof.

The compounds of the invention may be administered in the form of a pro-drug. A pro-drug is a bioprecursor or pharmaceutically acceptable compound being degradable in the body to produce a compound of the invention (such as an ester or amide of a compound of the invention, particularly an in vivo hydrolysable ester). Various forms of prodrugs are known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Academic Press, 1985);

b) A Textbook of Drug Design and Development, edited by Krogsgaard-Larsen;

c) H. Bundgaard, Chapter 5 “Design and Application of Prodrugs”, by H. Bundgaardp. 113-191 (1991);

d) H. Bundgaard, Advanced Drug Delivery Reviews, 8, 1-38 (1992);

e) H. Bundgaard, et al., Journal of Pharmaceutical Sciences, 77, 285 (1988); and

f) N. Kakeya, et al., Chem Pharm Bull, 32, 692 (1984).

The contents of the above cited documents are incorporated herein by reference.

Examples of pro-drugs are as follows. An in-vivo hydrolysable ester of a compound of the invention containing a carboxy or a hydroxy group is, for example, a pharmaceutically-acceptable ester which is hydrolysed in the human or animal body to produce the parent acid or alcohol. Suitable pharmaceutically-acceptable esters for carboxy include C₁ to C₆alkoxymethyl esters for example methoxymethyl, C₁ to C₆alkanoyloxymethyl esters for example pivaloyloxymethyl, phthalidyl esters, C₃ to ₈cycloalkoxycarbonyloxyC₁ to ₆alkyl esters for example 1-cyclohexylcarbonyloxyethyl; 1,3-dioxolen-2-onylmethyl esters, for example 5-methyl-1,3-dioxolen-2-onylmethyl; and C₁₋₆alkoxycarbonyloxyethyl esters.

An in-vivo hydrolysable ester of a compound of the invention containing a hydroxy group includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxy-methoxy. A selection of in-vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

A suitable pharmaceutically-acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically-acceptable salt of a benzoxazinone derivative of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

A further feature of the invention is a pharmaceutical composition comprising a compound of Formula (I) to (Ic) or (II) to (IIj) as defined above, or a salt, solvate or prodrug thereof, together with a pharmaceutically-acceptable diluent or carrier.

According to another aspect of the invention there is provided a compound of Formula (Ib) or (Ic), or (II) to (IIj) as defined above for use as a medicament; with the proviso that when R³ is hydrogen or methyl, m is 2 and n is 0 then (R¹)ₘ is other than di-C₁₋₄alkyl.

Further according to the invention there is provided a compound of Formula (Ib) or (Ic), or (II) to (IIj) for use in the preparation of a medicament for treatment of a disease mediated through GLK, in particular type 2 diabetes.

The compound is suitably formulated as a pharmaceutical composition for use in this way.

According to another aspect of the present invention there is provided a method of treating GLK mediated diseases, especially diabetes, by administering an effective amount of a compound of Formula (Ib) or (Ic), or (II) to (IIj) to a mammal in need of such treatment.

Specific disease which may be treated by the compound or composition of the invention include: blood glucose lowering in Diabetes Mellitus type 2 without a serious risk of hypoglycaemia (and potential to treat type 1), dyslipidemea, obesity, insulin resistance, metabolic syndrome X, impaired glucose tolerance.

Specific disease which may be treated by the compound or composition of the invention include: blood glucose lowering in Diabetes Mellitus type 2 (and potential to treat type 1); dyslipidaemia; obesity; insulin resistance; metabolic syndrome X; impaired glucose tolerance; polycystic ovary syndrome.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound of the Formula (I), (Ia), (Ib) or (Ic) will naturally vary according to the nature and severity of the conditions, the age and sex of the animal or patient and the route of administration, according to well known principles of medicine.

In using a compound of the Formula (I), (Ia), (Ib) or (Ic) for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

The elevation of GLK activity described herein may be applied as a sole therapy or may involve, in addition to the subject of the present invention, one or more other substances and/or treatments. Such conjoint treatment may be achieved by way of the simultaneous, sequential or separate administration of the individual components of the treatment. Simultaneous treatment may be in a single tablet or in separate tablets. For example in the treatment of diabetes mellitus chemotherapy may include the following main categories of treatment:

1) Insulin and insulin analogues;

2) Insulin secretagogues including sulphonylureas (for example glibenclamide, glipizide) and prandial glucose regulators (for example repaglinide, nateglinide);

3) Insulin sensitising agents including PPARg agonists (for example pioglitazone and rosiglitazone);

4) Agents that suppress hepatic glucose output (for example metformin).

5) Agents designed to reduce the absorption of glucose from the intestine (for example acarbose);

6) Agents designed to treat the complications of prolonged hyperglycaemia;

7) Anti-obesity agents (for example sibutramine and orlistat);

8) Anti-dyslipidaemia agents such as, HMG-CoA reductase inhibitors (statins, eg pravastatin); PPARα agonists (fibrates, eg gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations);

9) Antihypertensive agents such as, β blockers (eg atenolol, inderal); ACE inhibitors (eg lisinopril); Calcium antagonists (eg. nifedipine); Angiotensin receptor antagonists (eg candesartan), a antagonists and diuretic agents (eg. furosemide, benzthiazide);

10) Haemostasis modulators such as, antithrombotics, activators of fibrinolysis and antiplatelet agents; thrombin antagonists; factor Xa inhibitors; factor VIIa inhibitors); antiplatelet agents (eg. aspirin, clopidogrel); anticoagulants (heparin and Low molecular weight analogues, hirudin) and warfarin; and

11) Anti-inflammatory agents, such as non-steroidal anti-infammatory drugs (eg. aspirin) and steroidal anti-inflammatory agents (eg. cortisone).

According to another aspect of the present invention there is provided individual compounds produced as end products in the Examples set out below and salts thereof.

A compound of the invention, or a salt, pro-drug or solvate thereof, may be prepared by any process known to be applicable to the preparation of such compounds or structurally related compounds. Such processes are illustrated by the following representative schemes (1 and 2) in which variable groups have any of the meanings defined for Formula (I) unless stated otherwise. Functional groups may be protected and deprotected using conventional methods. For examples of protecting groups such as amino and carboxylic acid protecting groups (as well as means of formation and eventual deprotection), see T. W. Greene and P. G. M. Wuts, “Protective Groups in Organic Synthesis”, Second Edition, John Wiley & Sons, New York, 1991. Note abbreviations used have been listed immediately before the Examples below.

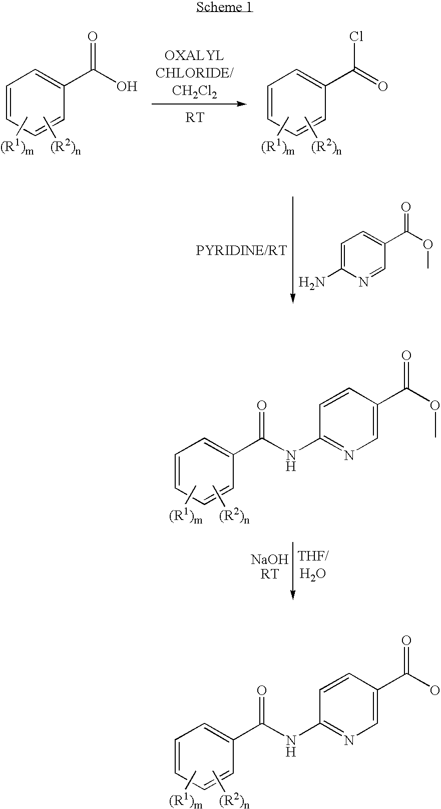

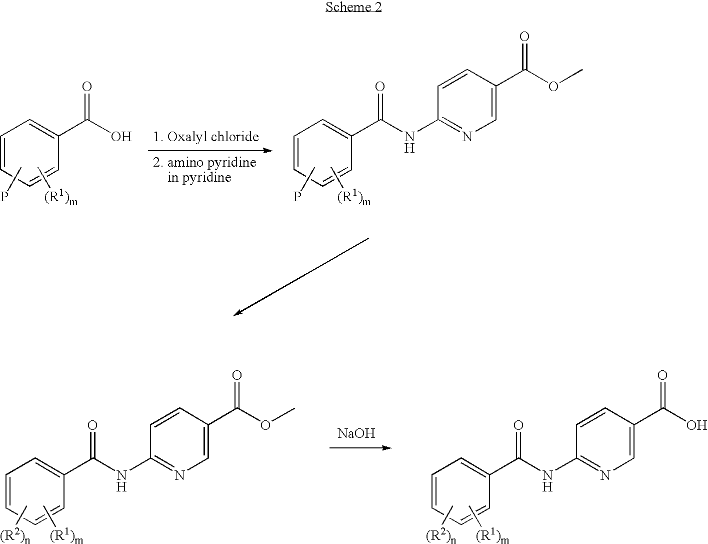

In Scheme 2 P represents a protecting group for a functional group within R² or alternatively P is a precursor group for conversion to a functional group or substituent R².

Processes for the synthesis of compounds of Formula (I) are provided as a further feature of the invention. Thus, according to a further aspect of the invention there is provided a process for the preparation of a compound of Formula (I) which comprises:

(a) reaction of a compound of Formula (IIIa) with a compound of Formula (IIIb),

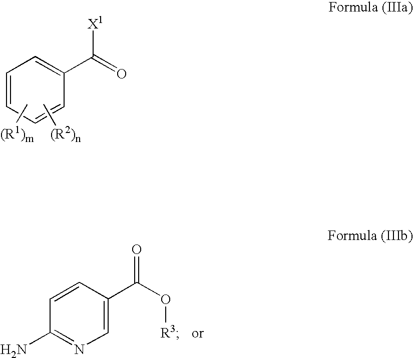

wherein X¹ is a leaving group

(b) for compounds of Formula (I) wherein R³ is hydrogen, de-protection of a compound of Formula (IIIc),

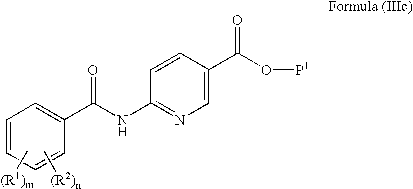

wherein P¹ is a protecting group;

(c) for compounds of Formula (I) wherein n is 1, 2, 3 or 4, reaction of a compound of Formula (IIId) with a compound of Formula (IIIe),

Y—X″ Formula (IIId)

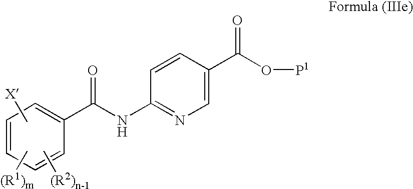

wherein X′ and X″ comprises groups which when reacted together form the group X;

(d) for a compound of Formula (I) wherein n is 1, 2, 3 or 4 and X or X¹ is —SO-Z- or —SO₂-Z-, oxidation of the corresponding compound of Formula (I) wherein X or X¹ respectively is —S-Z-;

(e) reaction of a compound of Formula (IIIf) with a compound of Formula (IIIg),

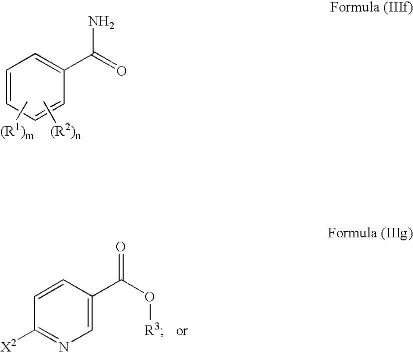

wherein X² is a leaving group

and thereafter, if necessary:

i) converting a compound of Formula (I) into another compound of Formula (I);

ii) removing any protecting groups;

iii) forming a salt, pro-drug or solvate thereof.

Specific reaction conditions for the above reactions are as follows:

Process a)—as described above;

Process b)—as described above;

Process c)—examples of this process are as follows:

(i) to form a group when X is —O-Z-, X′ is a group of formula HO-Z- and X″ is a leaving group (alternatively X′ is a group of formula L²-Z- wherein L² is a leaving group and X″ is a hydroxyl group), compounds of Formula (IIId) and (IIIe) are reacted together in a suitable solvent, such as DMF or THF, with a base such as sodium hydride or potassium tert-butoxide, at a temperature in the range 0 to 100° C., optionally using metal catalysis such as palladium on carbon or cuprous iodide;

(ii) to form a group when X is N(R⁶)-Z-, X′ is a group of formula H—(R⁶)N-Z- and X″ is a leaving group (alternatively X′ is a group of formula L²-Z- wherein L² is a leaving group and X″ is a group or formula —N(R⁶)—H), compounds of Formula (IIId) and (IIIe) are reacted together in a suitable solvent such as THF, an alcohol or acetonitrile, using a reducing agent such as sodium cyano borohydride or sodium trisacetoxyborohydride at room temperature;

(iii) to form a group when X is —SO₂N(R⁶)-Z-, X′ is a group of formula H—N(R⁶)-Z- wherein L² is a leaving group and X″ is an activated sulphonyl group such as a group of formula —SO₂—Cl, compounds of Formula (IIId) and (IIIe) are reacted together in a suitable solvent such as methylene chloride, THF or pyridine, in the presence of a base such as triethylamine or pyridine at room temperature;

(iv) to form a group when X is —N(R⁶)SO₂-Z-, X′ is an activated sulphonyl group such as a group of formula Cl—SO₂-Z- group and X″ is a group of formula —N(R⁶)-L² wherein L² is a leaving group, compounds of Formula (IIId) and (IIIe) are reacted together in a suitable solvent such as methylene chloride, THF or pyridine, in the presence of a base such as triethylamine or pyridine at room temperature;

(v) to form a group when X is —C(O)N(R⁶)-Z-, X′ is a group of formula H—N(R⁶)-Z- wherein L² is a leaving group and X″ is an activated carbonyl group such as a group of formula —C(O)—Cl, compounds of Formula (IIId) and (IIIe) are reacted together in a suitable solvent such as THF or methylene chloride, in the presence of a base such as triethylamine or pyridine at room temperature;

(vi) to form a group when X is —N(R⁶)C(O)-Z-, X′ is an activated carbonyl group such as a group of formula Cl—C(O)-Z- group and X″ is a group of formula —N(R⁶)-L² wherein L² is a leaving group, compounds of Formula (IIId) and (IIIe) are reacted together in a suitable solvent such as THF or methylene chloride, in the presence of a base such as triethylamine or pyridine at room temperature;

(vii) to form a group when X is —CH═CH-Z-, a Wittag reaction or a Wadsworth-Emmans Homer reaction can be used. For example, X′ terminates in an aldehyde group and Y—X″ is a phosphine derivative of the formula Y—C—H—P⁺PH₃ which can be reacted together in a strong base such as sodium hydride or potassium tert-butoxide, in a suitable solvent such as THF at a temperature between room temperature and 100° C.

Process d)—the oxidization of a compound of Formula (I) wherein X or X¹ is —S-Z- is well known in the art, for example, reaction with metachloroperbenzoic acid (MCPBA) is the presence of a suitable solvent such as dichloromethane at ambient temperature. If an excess of MCPBA is used a compound of Formula (I) wherein X is —S(O₂)— is obtained.

Process e)—reaction of a Formula (IIIf) with a compound of Formula (IIIg) can be performed in a polar solvent, such as DMF or a non-polar solvent such as THF with a strong base, such as sodium hydride or potassium tert-butoxide at a temperature between and 100° C., optionally using metal catalysis, such as palladium on carbon or cuprous iodide.

Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Specific examples of protecting groups are given below for the sake of convenience, in which “lower” signifies that the group to which it is applied preferably has 1-4 carbon atoms. It will be understood that these examples are not exhaustive. Where specific examples of methods for the removal of protecting groups are given below these are similarly not exhaustive. The use of protecting groups and methods of deprotection not specifically mentioned is of course within the scope of the invention.

A carboxy protecting group may be the residue of an ester-forming aliphatic or araliphatic alcohol or of an ester-forming silanol (the said alcohol or silanol preferably containing 1-20 carbon atoms). Examples of carboxy protecting groups include straight or branched chain (C₁₋₁₂)alkyl groups (e.g. isopropyl, t-butyl); lower alkoxy lower alkyl groups (e.g. methoxymethyl, ethoxymethyl, isobutoxymethyl; lower aliphatic acyloxy lower alkyl groups, (e.g. acetoxymethyl, propionyloxymethyl, butyryloxymethyl, pivaloyloxymethyl); lower alkoxycarbonyloxy lower alkyl groups (e.g. 1-methoxycarbonyloxyethyl, 1-ethoxycarbonyloxyethyl); aryl lower alkyl groups (e.g. p-methoxybenzyl, o-nitrobenzyl, p-nitrobenzyl, benzhydryl and phthalidyl); tri(lower alkyl)silyl groups (e.g. trimethylsilyl and t-butyldimethylsilyl); tri(lower alkyl)silyl lower alkyl groups (e.g. trimethylsilylethyl); and (2-6C)alkenyl groups (e.g. allyl and vinylethyl).

Methods particularly appropriate for the removal of carboxylprotecting groups include for example acid-, metal- or enzymically-catalysed hydrolysis.

Examples of hydroxy protecting groups include lower alkenyl groups (e.g. allyl); lower alkanoyl groups (e.g. acetyl); lower alkoxycarbonyl groups (e.g. t-butoxycarbonyl); lower alkenyloxycarbonyl groups (e.g. allyloxycarbonyl); aryl lower alkoxycarbonyl groups (e.g. benzoyloxycarbonyl, p-methoxybenzyloxycarbonyl, o-nitrobenzyloxycarbonyl, p-nitrobenzyloxycarbonyl); tri lower alkyl/arylsilyl groups (e.g. trimethylsilyl, t-butyldimethylsilyl, t-butyldiphenylsilyl); aryl lower alkyl groups (e.g. benzyl) groups; and triaryl lower alkyl groups (e.g. triphenylmethyl).

Examples of amino protecting groups include formyl, aralkyl groups (e.g. benzyl and substituted benzyl, e.g. p-methoxybenzyl, nitrobenzyl and 2,4-dimethoxybenzyl, and triphenylmethyl); di-p-anisylmethyl and furylmethyl groups; lower alkoxycarbonyl (e.g. t-butoxycarbonyl); lower alkenyloxycarbonyl (e.g. allyloxycarbonyl); aryl lower alkoxycarbonyl groups (e.g. benzyloxycarbonyl, p-methoxybenzyloxycarbonyl, o-nitrobenzyloxycarbonyl, p-nitrobenzyloxycarbonyl; trialkylsilyl (e.g. trimethylsilyl and t-butyldimethylsilyl); alkylidene (e.g. methylidene); benzylidene and substituted benzylidene groups.

Methods appropriate for removal of hydroxy and amino protecting groups include, for example, acid-, base, metal- or enzymically-catalysed hydrolysis, or photolytically for groups such as o-nitrobenzyloxycarbonyl, or with fluoride ions for silyl groups.

Examples of protecting groups for amide groups include aralkoxymethyl (e.g. benzyloxymethyl and substituted benzyloxymethyl); alkoxymethyl (e.g. methoxymethyl and trimethylsilylethoxymethyl); tri alkyl/arylsilyl (e.g. trimethylsilyl, t-butyldimethylsilyl, t-butyldiphenylsilyl); trialkyl/arylsilyloxymethyl (e.g. t-butyldimethylsilyloxymethyl, t-butyldiphenylsilyloxymethyl); 4-alkoxyphenyl (e.g. 4-methoxyphenyl); 2,4-di(alkoxy)phenyl (e.g. 2,4-dimethoxyphenyl); 4-alkoxybenzyl (e.g. 4-methoxybenzyl); 2,4-di(alkoxy)benzyl (e.g. 2,4-di(methoxy)benzyl); and alk-1-enyl (e.g. allyl, but-1-enyl and substituted vinyl e.g. 2-phenylvinyl).

Aralkoxymethyl, groups may be introduced onto the amide group by reacting the latter group with the appropriate aralkoxymethyl chloride, and removed by catalytic hydrogenation. Alkoxymethyl, tri alkyl/arylsilyl and tri alkyl/silyloxymethyl groups may be introduced by reacting the amide with the appropriate chloride and removing with acid; or in the case of the silyl containing groups, fluoride ions. The alkoxyphenyl and alkoxybenzyl groups are conveniently introduced by arylation or alkylation with an appropriate halide and removed by oxidation with ceric ammonium nitrate. Finally alk-1-enyl groups may be introduced by reacting the amide with the appropriate aldehyde and removed with acid.

The following examples are for illustration purposes and are not intended to limit the scope of this application. Each exemplified compound represents a particular and independent aspect of the invention. In the following non-limiting Examples, unless otherwise stated:

(i) evaporations were carried out by rotary evaporation in vacuo and work-up procedures were carried out after removal of residual solids such as drying agents by filtration;

(ii) operations were carried out at room temperature, that is in the range 18-25° C. and under an atmosphere of an inert gas such as argon or nitrogen;

(iii) yields are given for illustration only and are not necessarily the maximum attainable;

(iv) the structures of the end-products of the Formula (I) were confirmed by nuclear (generally proton) magnetic resonance (NMR) and mass spectral techniques; proton magnetic resonance chemical shift values were measured on the delta scale and peak multiplicities are shown as follows: s, singlet; d, doublet; t, triplet; m, multiplet; br, broad; q, quartet, quin, quintet;

(v) intermediates were not generally fully characterised and purity was assessed by thin layer chromatography (TLC), high-performance liquid chromatography (HPLC), infra-red (IR) or NMR analysis;

(vi) chromatography was performed on silica (Merck Silica gel 60, 0.040-0.063 mm, 230-400 mesh); and

(vi) Biotage cartridges refer to pre-packed silica cartridges (from 40 g up to 400 g), eluted using a biotage pump and fraction collector system; Biotage UK Ltd, Hertford, Herts, UK.

Abbreviations

| | |
|---|---|
| ADDP | azodicarbonyl)dipiperidine; |
| DCM | dichloromethane; |
| DEAD | diethyldiazocarboxylate; |
| DIAD | di-i-propyl azodicarboxylate; |
| DMSO | dimethyl sulphoxide; |
| DMF | dimethylformamide; |
| DtAD | di-t-butyl azodicarboxylate; |
| EDAC | 1-(3-dimethylaminopropyl)-3-ethylcarbodiimide |
| | hydrochloride; |
| HATU | O-(7-azabenzotriazol-1-yl)-N,N,N′,N′- |
| | tetramethyluronium hexafluorophosphate; |
| LCMS | liquid chromatography/mass spectroscopy; |
| MPLC | medium pressure liquid chromatography; |
| RT | room temperature; and |
| THF | tetrahydrofuran. |

### EXAMPLE A

### 6-[(3,5-Dibenzyloxybenzoyl)amino]-3-pyridinecarboxylic acid (Route 1)

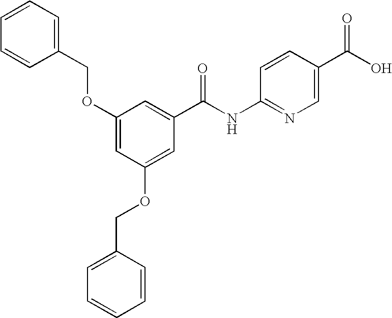

The methyl ester (267 mg 0.57 mM) of the title compound was stirred with lithium hydroxide (150 mg [excess]) in a mixture of tetrahydrofuran (THF) (10 ml) and water (1 ml) at room temperature overnight. The solvent was removed and water (10 ml) added. After acidification with 11.0M hydrochloric acid, to Ph=4, the precipitated solid was filtered off, washed with water and dried ‘in vacuo’. This gave the title compound (43 mg 17%); H¹ NMR δ (d₆-DMSO) 5.17 (4H s) 6.86 (1H s) 7.30-7.47 (12H m) 8.25 (2H s) 8.86 (1H s) 11.02 (1H b); MS [MH]⁺ 455

The methyl ester starting material was prepared as follows:

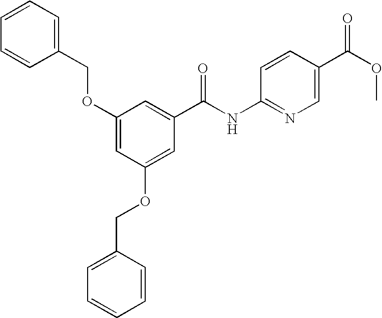

3,5-Dibenzyloxbenzoic acid (334 mg 1.0 mM) was suspended in methylene chloride with stirring. Oxalyl chloride (0.146 mg, 1.147 Mm) and N,N-dimethylformamide (DMF) (1 drop) were added and the mixture was stirred at room temperature for 2 hours. The solvent was removed and the residue was redissolved in methylene chloride (5 ml). This solution was then added to a suspension of methyl-6-aminonicotinate (152 mg 1.0 mM) in methylene chloride (5 ml) and pyridine (80 μl), after stirring at room temperature overnight the reaction mixture was partitioned between methylene chloride and saturated ammonium chloride, dried over magnesium sulphate, filtered and the solvent removed by distillation ‘in vacuo’ to give the crude product. This was purified by elution down a silica column using ethyl acetate/isohexane as solvent. This gave methyl 6-[(3,5-dibenzyloxybenzoyl)amino]3-pyridinecarboxylate as a white solid (267 mg 57%). MS [MH]⁺ 469

### EXAMPLE B

### 6-[(3,5-Di-(2-methylbenzyloxy)benzoyl)amino]-3-pyridinecarboxylic acid (Route 2)

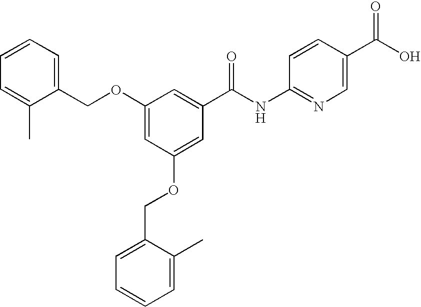

Methyl 6-[(3,5-di-(2-methylbenzyloxy)benzoyl)amino]-3-pyridinecarboxylate (61 mgs) was stirred at ambient temperature in a mixture of THF (4 ml), methanol (1 ml) and water (1 ml) with 2M sodium hydroxide (0.3 ml, xs). After four hours the solvent was removed, under reduced pressure, water (5 ml) added and the pH adjusted to neutral. This gave a white precipitate which was filtered off, washed with water, dried to give the title compound (56 mgs, 94%). MS [MH]⁺ 483

The starting methyl ester was prepared as follows:—

3,5-Diacetoxybenzoic acid (15 g, 63 mM) was suspended in dichloromethane (100 mls), THF(20 mls) with oxalyl chloride (7.34 mls, 69.3 mM) and DMF(2-3 drops) added. The resultant mixture was stirred for three hours at ambient temperature in a flask fitted with a gas bubbler. This gave a pale brown solution. After concentration ‘in vacuo’ the residue was triturated with diethyl ether. This gave a colourless solid, 3,5-diacetoxybenzoyl chloride (15.95 g) which was used for the next stage without further purification.

Diacetoxybenzoyl chloride (15.95 g, 62 mM) suspended in methylene chloride (3 ml) added to a solution of methyl 2-aminopyridine-5-carboxylate (9.57 g, 62 mM) dissolved in pyridine (5 ml). Resultant mixture stirred for 18 hrs at ambient temperature, pyridine azeotroped off with toluene and the residue purified by elution down a silica column using a 10:90 mixture of ethyl acetate:dichloromethane as eluent. This gave methyl 6-[(3,5-di-acetoxybenzoyl)amino]-3-pyridinecarboxylate (12.67 g); H¹ NMR δ (CDCl3) 3.95 (3H s), 7.19 (1H m), 7.58 (2H d), 8.39 (2H m), 8.70 (1H bs), 8.92(1H m)

Methyl 6-[(3,5-di-acetoxybenzoyl)amino]-3-pyridinecarboxylate (6 g, 16.1 mM) was stirred at ambient temperature in THF (50 ml) and sodium methoxide solution (14.8 ml of 25% in methanol, 64.4 mM) added slowly. The resultant solution was stirred for one hour, poured into 1M hydrochloric acid and the pH adjusted to pH=4 with sodium bicarbonate solution, extracted with ethyl acetate, extracts combined, washed with brine and dried over anhydrous magnesium sulphate. The solvent was removed by distillation under reduced pressure to give a yellow solid. This solid was triturated with hot methanol, filtered, to give methyl 6-[(3,5-dihydroxybenzoyl)amino]-3-pyridinecarboxylate as a pale yellow solid (3.51 g, 77%); H¹ NMR δ (d₆-DMSO) 3.85 (3H s) 6.41 (1H s) 6.80 (2H d)8.28 (2 Hm)8.85 (1 Hd)9.52 (2 H s)

Alpha-bromo-O-xylene (272 mgs, 1.5 mM), silver carbonate (402 mgs, 3.7 mM) and methyl 6-[(3,5-dihydroxybenzoyl)amino]-3-pyridinecarboxylate (200 mgs, 0.7 Mm) were stirred at ambient temperature in DMF (4 mls) for 18 hrs. The solvent was removed under reduced pressure, the residue dissolved in methylene chloride and purified by elution down a silica bond-elute column using methylene chloride/ethyl acetate as eluent. This gave methyl 6-[(3,5-di-(2-methylbenzyloxy)benzoyl)amino]-3-pyridinecarboxylate (61 mgs).

MS [MH]⁺ 497

### EXAMPLE C

### 6-{[3-(2-Methylbenzyloxy)-5-(5-methylisoxazol-3-ylmethoxy)benzoyl]amino}-3-pyridinecarboxylic acid (Route 3)

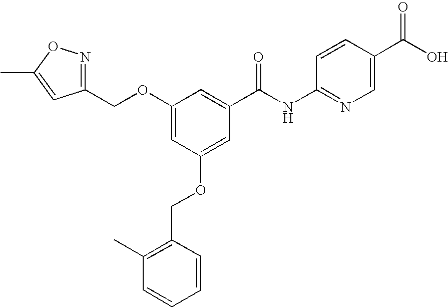

Methyl 6-{[3-(2-methylbenzyloxy)-5-(5-methylisoxazol-3-ylmethoxy)benzoyl]amino}-3-pyridinecarboxylate (98 mg, 0.201 mM) was dissolved in THF (4 ml) and a solution of NaOH (24 mg, 0.603 mM) in water (0.24 ml) was added. Water (4 ml) was added to the reaction mixture until it became monophasic. The reaction was stirred for 16 hours at ambient temperature and was then acidified to pH=1 with 1N aqueous HCl. The white solid which precipitated from the mixture was isolated by filtration and was dried ‘in vacuo’ to give the title compound as a white solid (67 mg, 70% yield); H¹ NMR δ (d⁶-DMSO) 2.30 (3H s) 2.39 (3H s) 5.16 (2H s) 5.22 (2H s) 6.33 (1H s) 6.91 (1H s) 7.11-7.42 (6H m) 8.30 (2H s) 8.87 (1H s). MS [MH]⁺ 474

The starting material was prepared as follows:

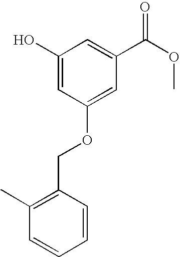

To a solution of methyl 3,5-dihydroxybenzoate (50 g, 0.30M) in N,N-dimethylformamide (500 ml) at 0° C. was added sodium hydride (10.8 g, 0.27M) portionwise, maintaining the reaction temperature below 10° C. The reaction was allowed to warm to 15° C. and was stirred for 20 minutes. The mixture was cooled to 0° C. and a solution of 2-methylbenzyl bromide (36 ml, 0.27M) in N,N-dimethylformamide (50 ml) was added over 30 minutes. The reaction was warmed to ambient temperature and concentrated ‘in vacuo’. Ethyl acetate (500 ml) was added to the residue and the resulting organic solution was washed first with water (2×250 ml) and then with a saturated aqueous sodium chloride solution (200 ml). The organic layer was dried with magnesium sulfate and then concentrated ‘in vacuo’. The crude product was chromatographed on Kieselgel 60, eluting with a gradient of 0-100% ethyl acetate in iso-hexane to give methyl 3-hydroxy-5-(2-methylbenzyloxy)-benzoate as a colourless solid (21.9 g); H¹ NMR δ (d⁶-DMSO) 2.39 (3H s) 3.90 (3H s) 5.02 (2H s) 5.61 (1H s) 6.69 (1H t) 7.15-7.42 (6H, m). MS [MH]⁺ 488

The starting material was prepared as follows:

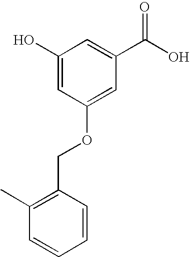

To a solution of methyl 3-hydroxy-5-(2-methylbenzyloxy) benzoate (21.72 g, 79.9 mM) in methanol (480 ml) and water (167 ml) was added 2M sodium hydroxide (160 ml, 320 mM). The reaction was stirred for 2 hours at ambient temperature and then for 1 hour at 60° C. The mixture was reduced ‘in vacuo’ to ⅓ volume and was acidified with 2N aqueous HCl which resulted in the precipitation of a white solid. The mixture was filtered and the solid was washed with water before being dried ‘in vacuo’ to give 3-hydroxy-5-(2-methylbenzyloxy) benzoic acid as a white solid (19.92 g).

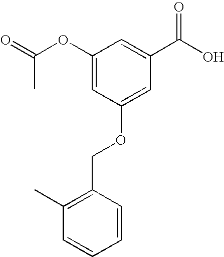

3-Hydroxy-5-(2-methylbenzyloxy) benzoic acid (20.30 g, 78.6 mM) and acetic anhydride (125 ml, 1.32M) in acetic acid (125 ml) were refluxed for 16 hours. The reaction was cooled and the solvent evaporated ‘in vacuo’. Acetic acid (125 nml) and water (125 ml) were added to the resulting residue and the mixture was stirred for 1 hour at 50° C. Toluene (100 ml) was added and the solvent distilled off ‘in vacuo’ to give 3-acetoxy-5-(2-methylbenzyloxy) benzoic acid as a colourless solid (23.6 g); H¹ NMR δ (d⁶-DMSO) 2.25 (3H s) 2.32 (3H s) 5.12 (2H s) 7.09-7.25 (7H, m).

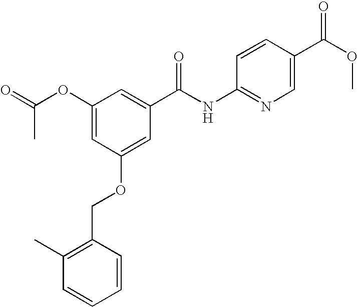

To a solution of 3-acetoxy-5-(2-methylbenzyloxy) benzoic acid (12 g, 40 mM) in methylene chloride (125 ml) was added oxalyl chloride (3.8 ml, 44 mM). N,N-dimethylformamide (5 drops) was then added slowly to the reaction mixture followed by THF (20 ml). The reaction was stirred for 2 hours before the solvent was removed under reduced pressure. Toluene (100 ml) was added and the resulting mixture was again concentrated to give a brown solid to which was added DCM (100 ml). The resulting solution was added to a mixture of methyl-6-amino-nicotinate (5.78 g, 38 mM) in pyridine (140 ml) and the reaction was stirred for 16 hours at ambient temperature. The reaction was concentrated under reduced pressure and ethyl acetate (100 ml) and water (100 ml) w ere added to the resulting brown residue. This mixture was sonicated and filtered to give a colourless solid which was washed with ethyl acetate (50 ml) and water (50 ml). The solid was then dried under reduced pressure to yield the product as a colourless solid (10.65 g). The filtrates were separated and the organic phase was reduced under reduced pressure and the resulting residue was purified by flash column chromatography eluting with a gradient of 0-5% ethyl acetate in methylene chloride to give methyl 6-{[3-acetoxy-5-(2-methylbenzyloxy)benzoyl]amino}-3-pyridinecarboxylate as a colourless solid (1.24 g) which was combined with previously obtained precipitate to give total yield (11.89 g); H¹ NMR δ (d⁶-DMSO) 2.25 (3H s) 2.31 (3H s) 3.85 (3H s) 5.19 (2H s) 7.04-7.12 (1H m) 7.15-7.30 (3H m) 7.39-7.45 (2H m) 7.65 (1H s) 8.31 (2H s) 8.91 (1H s). LCMS [M+H] 435, [M−H] 433.

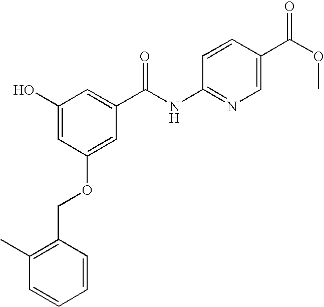

Methyl 6-{[3-acetoxy-5-(2-methylbenzyloxy)benzoyl]amino}-3-pyridinecarboxylate (11.64 g, 26.8 mM) was dissolved in THF (150 ml) and sodium methoxide (25% in methanol) (11.6 ml, 53.6 mM) was added. The resulting yellow solution was stirred for 20 minutes at ambient temperature and was then added to dilute hydrochloric acid. The pH of the mixture was adjusted to pH=4 by the addition of sodium bicarbonate and acetic acid before ethyl acetate (50 ml) and water (25 ml) were added. This resulted in the precipitation of a colourless solid which was isolated by filtration and washed with water and ethyl acetate before being dried over magnesium sulphate, filtered, to give methyl 6-{[3-hydroxy-5-(2-methylbenzyloxy)benzoyl]amino}-3-pyridinecarboxylate as a colourless solid (9.62 g); H¹ NMR δ (d⁶-DMSO) 2.33 (3H s) 3.85 (3H s) 5.11 (2H s) 6.61 (1H s) 7.01 (1H s) 7.18-7.29 (4H m) 7.40 (1H d) 8.32 (2H s) 8.90 (1H s) 9.77 (1H s) 11.04 (1H s).

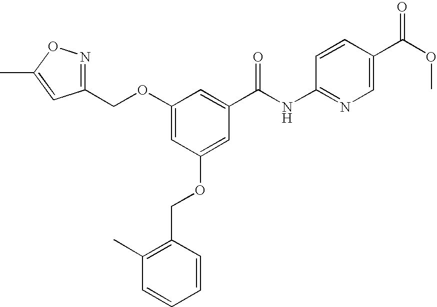

Methyl 6-{[3-hydroxy-5-(2-methylbenzyloxy)benzoyl]amino}-3-pyridinecarboxylate (150 mg, 0.38 mM), potassium iodide (13 mg, 0.08 mM) and potassium carbonate (56 mg, 0.41 mM) in acetone (3 ml) were heated to 55° C. and a solution of 3-chloromethyl-5-methyl isoxazole (55 mg, 0.421 mM) in acetone (2 ml) was added. The reaction was stirred for 1 hour at 55° C. and a further addition of 3-chloromethyl-5-methyl isoxazole (33 mg, 0.25 mM) in acetone (1 ml) was made. The reaction was stirred for 24 hours at 55° C. before being allowed to cool to ambient temperature. Ethyl acetate (15 ml) was added and the resulting mixture was washed with 1N aqueous HCl (10 ml), saturated aqueous sodium bicarbonate solution (10 ml) and water (10 ml). The solvent was removed under reduced pressure to give methyl 6{[3-(2-methylbenzyloxy)-5-(5-methylisoxazol-3-ylmethoxy)benzoyl]amino}-3-pyridinecarboxylate as a white solid (252 mg); H¹ NMR δ (d⁶-DMSO) 2.24 (3H s) 2.26 (3H s) 3.85 (3H s) 5.08 (2H s) 5.15 (s 2H) 6.28-6.35 (1H m) 6.88 (1H s) 7.17-7.43 (7H, m), 8.29 (1H s), 8.9 (11H d). MS [MH]+488

### EXAMPLE D

### 6-[(3-isobutoxy-5-isopropoxybenzoyl)amino]-3-pyridinecarboxylic acid (Route 4)

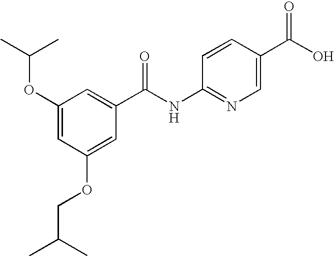

Methyl 6-[(3-isobutoxy-5-isopropoxybenzoyl)amino]-3-pyridinecarboxylate (230 mg, 0.62 mM) was dissolved in THF (8 ml) and a 2M NaOH solution (1.2 ml, 2.40 mM) was added. Water (7 ml) was added to the reaction mixture until it became monophasic. The reaction was stirred for 6 hours at ambient temperature and was then acidified to pH=1 with 1N aqueous HCl. The white solid which precipitated from the mixture was isolated by filtration and dried to give the title compound as a colourless solid (195 mg); H¹ NMR δ (d⁶-DMSO) 0.99 (6H d) 1.12 (6H d) 2.00 (1H sept) 3.80 (2H d) 4.65 (1H sept) 6.62 (1H s) 7.19 (2H s) 8.30 (2H s) 8.86 (1H s) 11.09 (1H s br); [M+H]373; [M−H] 371.

Preparation of the starting methyl ester was by the following stages:

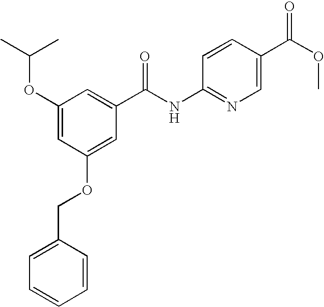

Methyl 6-[(3-benzyloxy-5-hydroxybenzoyl)amino]-3-pyridinecarboxylate(2.20 g, 5.81 mM), triphenylphosphine (1.59 g, 6.10 mM), iso-propanol (0.445 ml, 5.81 mM) and THF (50 ml) were combined and diisopropylazodicarboxylate (1.2 ml, 6.10 mM) was added dropwise. The reaction was stirred for 72 hours at ambient temperature. The mixture was concentrated in vacuo and the resulting brown oil was purified by column chromatography on Kieselgel 60, eluting with a gradient of 50-100% methylene chloride in iso-hexane and then 5% EtOAc in methylene chloride to give methyl 6-[(3-benzyloxy-5-isopropoxybenzoyl)amino]-3-pyridinecarboxylate as a colourless oil (1.92 g); H¹ NMR δ (d⁶-CDCl₃) 1.36 (6H d) 3.95 (3H s) 4.60 (1H sept) 5.09 (2H s) 6.72 (1H s) 7.02 (1H s) 7.10 (1H s) 7.30-7.50 (4H m) 8.39 (2H ddd) 8.68 (1H s br) 8.92 (1H s). [M+H] 421; [M-H] 419.

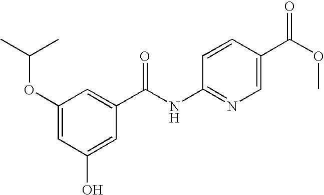

Methyl 6-[(3-benzyloxy-5-isopropoxybenzoyl)amino]-3-pyridinecarboxylate (1.92 g, 4.57 mM) was dissolved in THF (100 ml) and then ethanol (100 ml) and 10% palladium on carbon (250 mg) were added. The reaction was stirred at ambient temperature under an atmosphere of hydrogen (balloon) for 20 hours and was then filtered through diatomaceous earth. The filtrates were concentrated under reduced pressure to give methyl 6-[(3-hydroxy-5-isopropoxybenzoyl)amino]-3-pyridinecarboxylate as a colourless solid (1.42 g); H¹ NMR δ (d⁶-DMSO) 1.24 (6H d) 3.85 (3H s) 4.62 (1H sept) 6.49 (1H s) 6.97 (1H s) 7.04 (1H s) 8.30 (2H s) 8.89 (1H s) 9.67 (1H s) 11.01 (1H s br); [M+H]-331; [M−H] 329.

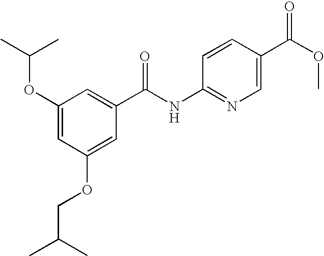

Methyl 6-[(3-hydroxy-5-isopropoxybenzoyl)amino]-3-pyridinecarboxylate (0.300 g, 0.91 mM), triphenylphosphine (0.238 g, 0.91 mM), iso-butanol (0.084 ml, 0.91 mM) and THF (8 ml) were combined and diisopropylazodicarboxylate (0.18 ml, 0.91 mM) was added dropwise. The mixture was stirred for 15 mins at ambient temperature. The reaction was concentrated under reduced pressure and the resulting brown oil was purified by column chromatography on Kieselgel 60, eluting with a gradient of 50-100% methylene chloride in iso-hexane and then 20% ethyl acetate in methylene chloride to give methyl 6-[(3-isobutoxy-5-isopropoxybenzoyl)amino]-3-pyridinecarboxylate as a colourless solid (0.232 g); [M+H]⁺ 387; [M−H]⁻ 385.

### EXAMPLE E

### 6-{[3,5-Di-(2-methylbenzoylamino)benzoyl]amino}-3-pyridinecarboxylic acid (Route 5)

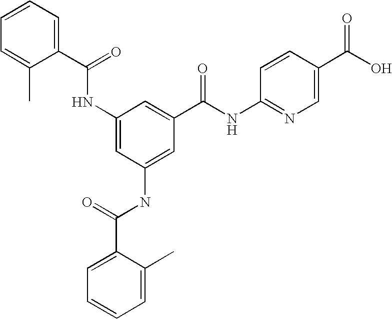

Methyl 6-{[3,5-di-(2-methylbenzoylamino)benzoyl]amino}-3-pyridinecarboxylate (130 mg 0.25 mM) was stirred at room temperature overnight with lithium hydroxide (52.5 mg 1.25 mM) in water (2 ml) and THF (10 ml). The mixture was then evaporated to remove the THF and acidified with 1.0N hydrochloric acid to pH=3. The precipitated solid was filtered, washed with water and vacuum dried at room temperature (70 mg 72.1%). Recrystallisation from ethyl acetate/methanol gave the title compound (16 mg 16.5%).

H¹ NMR δ (d⁶-DMSO) 2.52 (6H s) 7.32 (4H m) 7.42 (2H m) 7.52 (2H m) 8.08 (2H s)8.37(2Hs)8.48(1Hs)8.91 (1 Hs) 10.53(2Hs) 11.13(1Hs) 13.2(1Hb); MS [MH]⁺ 509.

The methyl ester intermediate was prepared by the following method:

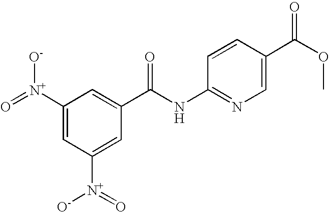

3,5-Dinitrobenzoic acid (4.24 g 20 mM) was stirred with oxalyl chloride (3.5 ml, xs) in methylene chloride (50 ml) and DMF (1 drop) at room temperature for 4 hours. The mixture was evaporated and then redissolved in methylene chloride (20 ml). This solution was added to a solution of methyl-6-aminonicotinate (3.0 g 20 mM) in pyridine (100 ml). After stirring at room temperature overnight the pyridine was evaporated off and the residue was chromatographed on silica using v/v ethyl acetate/isohexane to give methyl 6-[(3,5-dinitrobenzoyl)amino]-3-pyridinecarboxylate (5.2 g 75%). H¹ NMR δ (d⁶-DMSO)3.9(3H s) 8.35 (2H q) 8.95 (2H m) 9.18 (2 Hs)

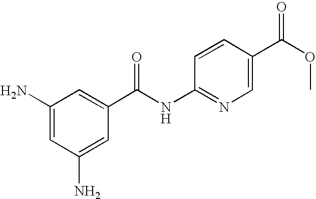

Methyl 6-[(3,5-dinitrobenzoyl)amino]-3-pyridinecarboxylate (4.9 g 14 mM) was dissolved in THF and 10% Pd/C (800 mg) was added. The mixture was hydrogenated until the uptake was complete and then filtered through diatomaceous earth. Evaporation of the filtrate gave a solid product (1.0 g). Further washing of the filter cake with large volumes of THF gave a further yield (850 mg) giving give methyl 6-[(3,5-diaminobenzoyl)amino]-3-pyridinecarboxylate as total weight of 1.85 g (46%); H¹ NMR δ (d⁶-DMSO) 3.85 (3H s 4.93 (4H bs) 6.0 (1H s) 6.38 (2H s)8.28 (2H m) 8.85 (1H s)10.41 (1H bs); MS [MH]⁺ 287

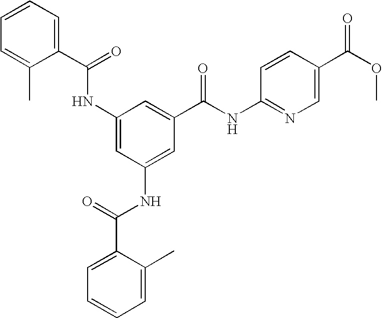

Methyl 6-[(3,5-diaminobenzoyl)amino]-3-pyridinecarboxylate (286 mg, 1 mM) was stirred at room temperature with 2-methylbenzoic acid (248 mg, 1.8 mM), HATU (950 mg, 2.5 mM) and di-isopropylethylamine (1.4 ml, 8 mM) in DMF (20 ml). The mixture was stirred overnight at room temperature and then poured into water and extracted with ethyl acetate. The extracts were dried (magnesium sulphate) filtered and evaporated to give an oil. Chromatography on silica using a gradient of ethyl acetate/hexane to give methyl 6-{[3,5-di-(2-methylbenzoylamino)benzoyl]amino}-3-pyridinecarboxylate (130 mg, 25%); H¹ NMR δ (d₆-DMSO) 2.5 (6H s) 3.9 (3H s) 7.25-7.55 (8H m) 8.05 (2H s) 8.3-8.45(3H mn) 8.9 (1H s) 10.55 (2H s) 11.2 (1 H s); MS [MH]⁺ 523

### EXAMPLE F

### 6-{1[35-diphenoxymethylbenzoyl]amino}-3-pyridinecarboxylic Acid (Route 6)

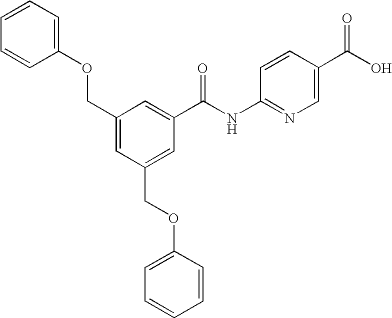

Methyl 3,5-diphenoxymethylphenylcarbamoyl pyridine-3-carboxylate (225 mg, 0.46 mM) was stirred at ambient temperature with 2.0M sodium hydroxide (1.2 ml, 2.4 mM), in water (10 ml) and THF (25 ml), overnight. After evaporating to half volume the mixture was acidified with dilute hydrochloric acid to give a precipitate. The precipitate was filtered off, washed with water and dried under vacuum to give a solid. This product was stirred in methanol (20 ml) at reflux, cooled, filtered and dried under vacuum to give the title compound as a colourless solid (148 mg 68%); H¹ NMR δ (d₆-DMSO) 5.2 (4H s) 6.95 (2H t) 7.05 (4H d) 7.3 (4H t) 7.78 (1H s) 8.1 (2H s) 8.3 (2H s) 8.88 (1H s) 11.2 (1H s) 13.25 (1H b); MS [MH]⁺ 455.

The starting methyl ester intermediate was prepared as follows:

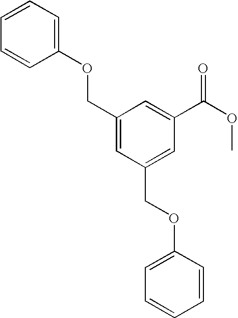

Methyl 3,5-dihydroxymethylbenzoate (500 mg 2.55 mM), triphenylphosphine (2.0 g 7.65 mM) and phenol (480 mg 5.1 mM) were dissolved in THF (20 ml) at ambient temperature. Di-isopropylazodicarboxylate (1.5 ml 7.65 mM) was added dropwise over 30 minutes. After stirring for a further 10 minutes the mixture was concentrated in vacuo and the residue was purified using MPLC (using silica and isohexane/dichloromethane as eluant) to give methyl 3,5-diphenoxymethylbenzoate as a colourless solid (534 mg 60%); H¹ NMR δ (d₆-DMSO) 3.92 (3H s) 5.1 (4H s) 6.92-7.02 (6H m) 7.12-7.36 (4H m) 7.72 (1H s) 8.07 (2H s); MS [MH]⁻ 347

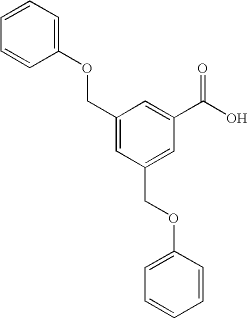

Methyl 3,5-diphenoxymethylbenzoate (525 mg 1.51 mM) 2.0M sodium hydroxide (2.3 n 4.6 mm) methanol (5 ml) water (3 ml) and THF (10 ml) were stirred together at room temperature for 3 hours. After concentrating to ½ volume the mixture was acidified with 2.0M hydrochloric acid and partitioned between ethyl acetate and water. The organic extracts were washed with water, dried (magnesium sulphate) filtered and evaporated to give 3,5-diphenoxymethylbenzoic acid as a colourless solid (500 mg, 99%); H¹ NMR δ (d₆-DMSO) 5.19 (4H s) 6.9-7.18 (6H m) 7.28 (4H t) 7.78 (1H s) 7.95 (2H s); MS [MH]⁻ 333.

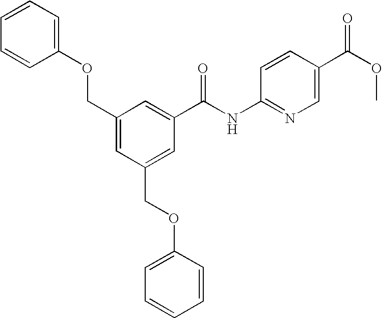

3,5-Diphenoxymethylbenzoic acid (500 mg 1.49 mM) was stirred with oxalyl chloride (1.4 ml 1.65 mM) in dichloromethane (20 ml) and DMF (1 drop) for 2 hours at ambient temperature. The solvent was removed by azeotroping with a small volume of toluene. The residue was dissolved in dichloromethane (10 ml) and added to a solution of methyl-6-aminonicotinate (250 mg 1.65 mM) in pyridine. The mixture was stirred at ambient temperature for 30 minutes and then the solvent evaporated to leave a brown residue. This was purified by MPLC on silica using ethyl acetate/isohexane as eluent This gave methyl 6-{[3,5-diphenoxymethylbenzoyl]amino}-3-pyridinecarboxylate (273 mg, 39%); H¹ NMR δ (d₆-DMSO) 3.95 (3H s) 5.15 (4H s) 6.96-7.05 (6H m) 7.21-7.29 (4H m) 7.75 (1H s) 7.95 (2H s) 8.3-8.52 (2H m) 8.9 (1H s) 8.93 (1H s)

### EXAMPLE G

### 2-{(3-amino-5-[2-(4-methyl-thiazol-5-yl) ethoxy]benzoylamino}-5-pyridine Carboxylic Acid (Route 7)

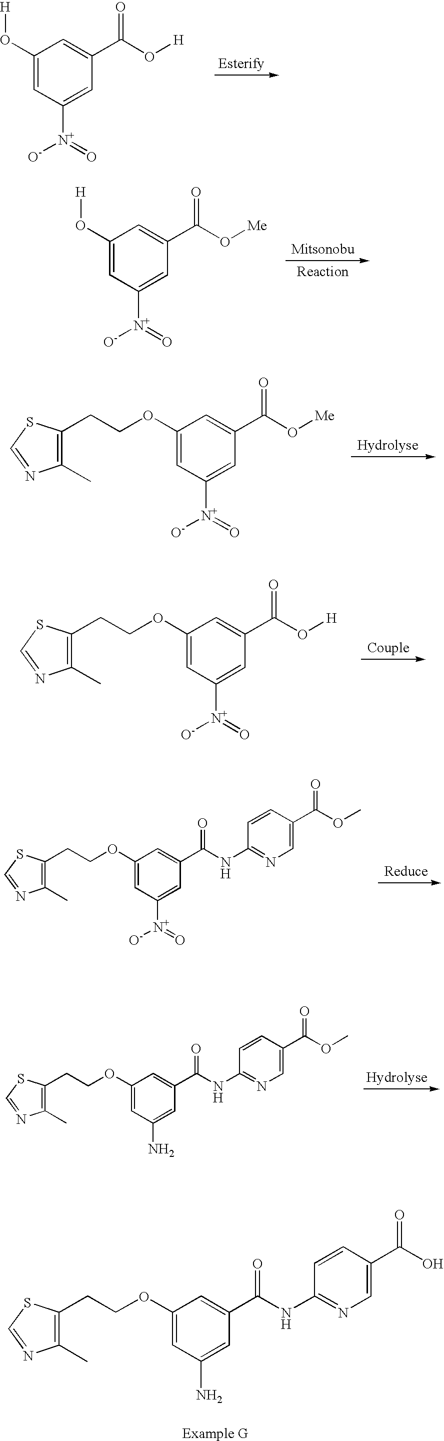

2M NaOH (1.5 ml, 3 mM) was added to a solution of methyl 6-[3-amino-5-(4-methyl-thiazol-5-yl) ethoxy]-3-pyridine carboxylate (0.40 g, 0.97 mM) in THF (30 ml)/water (30 ml). After 1 hr the reaction mixture was neutralised with 2M HCl then concentrated in vacuo. The pH was adjusted to 3-4 with 2M HCl, filtered, dried under high vacuum to give the title compound as a pale yellow solid (0.32 g, 83%); ¹H NMR δ (d₆-DMSO): 2.34 (s, 3H), 3.18 (dd, 2H), 4.13 (dd, 2H), 6.31 (m, 1H), 6.80 (m, 2H), 8.25 (s, 2H), 8.82 (s, 1H), 8.85 (s, 1H), 10.80 (bs, 1H).

The starting methyl ester intermediate was prepared as follows:

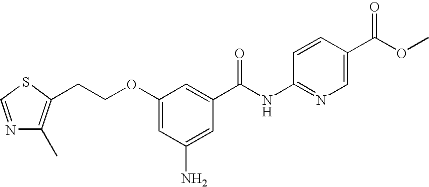

10% Palladium on carbon (0.20 g) was added under an argon atmosphere to a solution of methyl 2-[3-nitro-5-(4-methyl-thiazol-5-yl) ethoxy benzoyl]amino-5-pyridine carboxylate (1.05 g, 1.7 mM) in ethyl acetate (50 ml)/ethanol (50 ml). Hydrogen gas was introduced and the reaction mixture stirred vigorously for 18 hrs before filtering through diatomaceous earth, concentration in vacuo and replacement of the catalyst (80 mg). After stirring under hydrogen gas for a further 18 hrs a final catalyst change was carried out, after which the crude aniline was purified on silica gel (1% to 4% MeOH/DCM) to give the title compound as a colourless solid (0.43 g, 60%); ¹H NMR δ (d₆-DMSO): 2.36 (s, 3H), 3.18 (dd, 2H), 3.88 (s, 3H), 4.12 (dd, 2H), 5.32 (bs, 2H), 6.33 (m, 1H), 6.79 (m, 2H), 8.30 (m, 2H), 8.81 (s, 1H), 8.88 (m, 1H), 10.90 (bs, 1H).

The starting methyl 2-[3-nitro-5-(4-methyl-thiazol-5-yl) ethoxy benzoyl]amino-5-pyridine carboxylate was prepared according to the oxalyl chloride coupling method starting from 3-nitro-5-(4-methyl-thiazol-5-yl) ethoxy] benzoic acid, described in Example A:

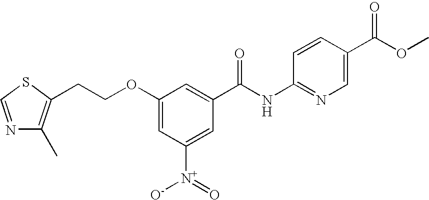

¹H NMR δ (d₆-DMSO): 2.35 (s, 3H), 3.28 (m, 2H), 3.87 (s, 3H), 4.37 (dd, 2H), 7.87 (m, 1H), 8.03 (m, 1H), 8.33 (m, 2H), 8.38 (m, 1H), 8.82 (s, 1H), 8.91 (m, 1H), 11.59 (bs, 1H).

The required 3-nitro-5-(4-methyl-thiazol-5-yl) ethoxy] benzoic acid was prepared by standard methodology starting from 3-nitro-5-hydroxy benzoic acid, according to the following scheme:

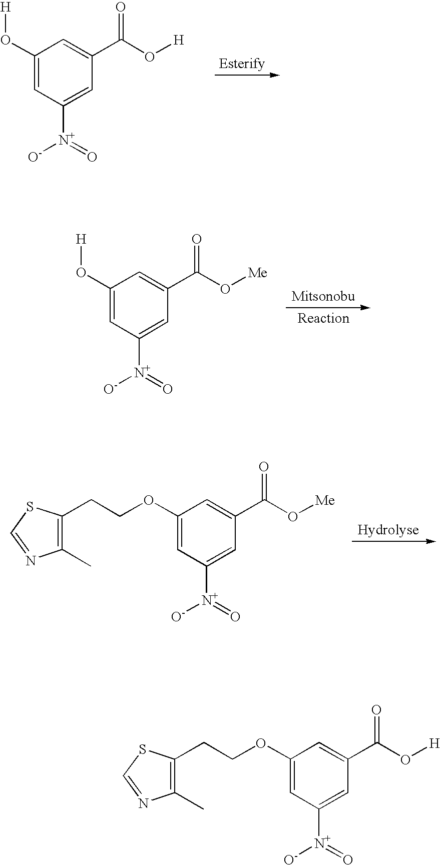

DIAD (3.16 ml, 16.1 mM) was added to a stirred solution of methyl 3-nitro-5-hydroxy benzoate (2.11 g, 10.7 mM), 2-(4-methylthiazol-5-yl) ethanol (1.55 ml, 12.8 mM) and triphenylphosphine (4.21 g, 16.1 mM) in THF (50 ml) under an argon atmosphere at room temperature. After 1 hr reaction mixture concentrated in vacuo, and the residue triturated with diethyl ether to give a colourless solid (triphenylphosphine oxide). Diethyl ether conc. to give a dark brown gum, purification on silica gel (50% to 75% EtOAc/iso-hexane) gave the product contaminated with reduced DIAD and triphenylphosphine oxide (6.8 g). The crude product was dissolved/suspended in MeOH (80 ml), 2M NaOH (20 ml, 40 mM) added, heated at 65° C. for 4 hrs then cooled and concentrated. The residue was diluted with water (140 ml)/2M NaOH (40 ml), the precipitated triphenylphosphine oxide filtered, then acidified with c. HCl to pH=1-2. The precipitate was filtered, washed with water, dried under high-vacuum to give 3-nitro-5-(4-methyl-thiazol-5-yl) ethoxy] benzoic acid as a colourless solid (3.12 g, 79% over 2 steps); ¹H NMR δ (d₆-DMSO): 2.39 (s, 3H), 3.23 (t, 2H), 4.35 (t, 2H), 7.78 (s. 1H), 7.90 (m, 1H), 8.22 (s, 1H), 8.93 (s, 1H).

### EXAMPLE H

### 2-{3-dimethylamino-5-[2-(4-methyl-thiazol-5-yl)ethoxy]benzoylamino}-5-pyridine Carboxylic Acid (Route 8)

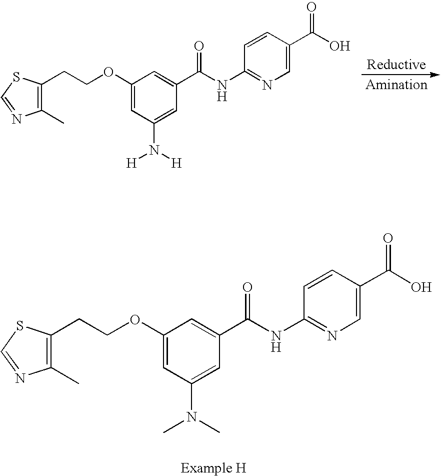

Formaldehyde (37% wt. in water) (0.021 ml, 0.75 mM) was added to a solution of 2-[3-amino-5-(4-methyl-thiazol-5-yl) ethoxy benzoyl]amino-5-pyridine carboxylic acid (0.10 g 0.25 mM) and 4A molecular sieves (0.25 g) in methanol (15 ml), under an inert atmosphere at room temperature. After 1 hr sodium cyanoborohydride (0.019 g, 0.3 mM) was added and the reaction mixture stirred for 40 hrs. The reaction mixture was filtered, concentrated in vacuo, 2M NaOH added to pH=11-12 then acidified with 2M HCl to precipitate a solid. The solid was filtered, washed with water, dried and purified on silica gel (5% to 12% MeOH/DCM) to give the title compound as a pale yellow solid (0.020 g, 19%); ¹H NMR δ (d₆-DMSO): 2.36 (s, 3H), 2.95 (m, 2H), 4.19 (dd, 2H), 6.39 (s, 1H), 6.92 (m, 2H), 6.99 (s, 1H), 8.27 (s, 2H), 8.83 (s, 1H), 8.88 (s, 1H), 11.02 (bs, 1H).

The 2-[3-amino-5-(4-methyl-thiazol-5-yl) ethoxy benzoyl]amino-5-pyridine carboxylic acid starting material was prepared as described in Example G.

### EXAMPLE I

### 2-{3-(2-methylbenzylamino)-5-[2-(4-methyl-thiazol-5-yl) ethoxy]benzoylamino}-5-pyridine Carboxylic Acid (Route 9)

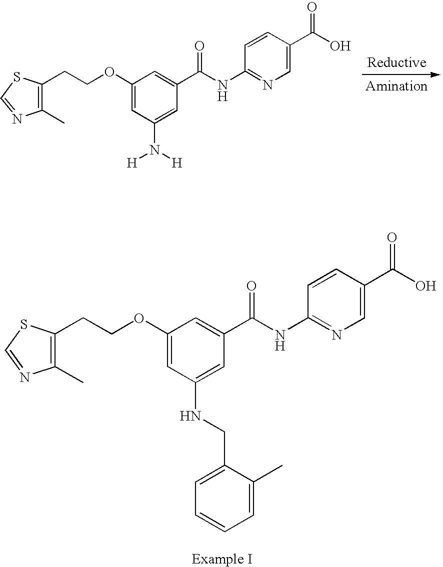

2-Methylbenzaldehyde (0.035 ml, 0.3 mM) was added to a solution of 2-[3-amino-5-(4-methyl-thiazol-5-yl) ethoxy benzoyl]amino-5-pyridine carboxylic acid (0.10 g 0.25 mM) and 4A molecular sieves (0.25 g) in methanol (15 ml), under an inert atmosphere at room temperature. After 1 hr sodium cyanoborohydride (0.019 g, 0.3 mM) was added and the reaction mixture stirred for 40 hrs. The reaction mixture was filtered, concentrated in vacuo, 2M NaOH added to pH=11-12 then acidified with 2M HCl to precipitate a colourless solid. The solid was filtered, washed with water to give the title compound as a colourless solid (0.12 g, 96%); ¹H NMR δ (d₆-DMSO): 2.33 (m, 6H), 3.19 (dd, 2H), 4.13 (dd, 2H), 4.26 (s, 2H), 6.33 (s, 1H), 6.83 (s, 1H), 6.90 (s, 1H), 7.09-7.19 (m, 3H), 7.26 (s, 1H), 8.28 (s, 2H), 8.83 (s, 1H), 8.88 (s, 1H), 10.87 (s, 1H), 13.09 (bs, 1H).

The 2-[3-amino-5-(4-methyl-thiazol-5-yl) ethoxy benzoyl]amino-5-pyridine carboxylic acid starting material was prepared as described in Example G.

### EXAMPLE J

### 2-[3-isopropyloxy-5-{(2-fluorophenoxy)methyl}benzoylamino]-5-pyridine Carboxylic Acid (Route 10)

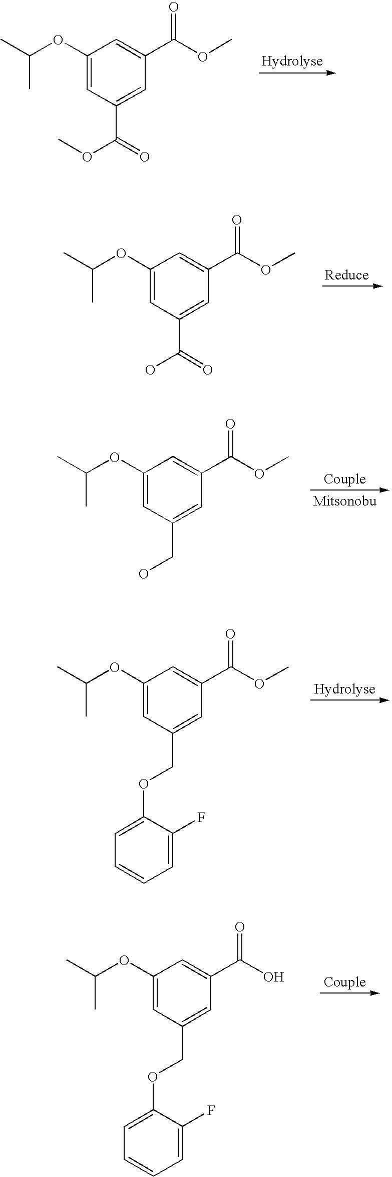

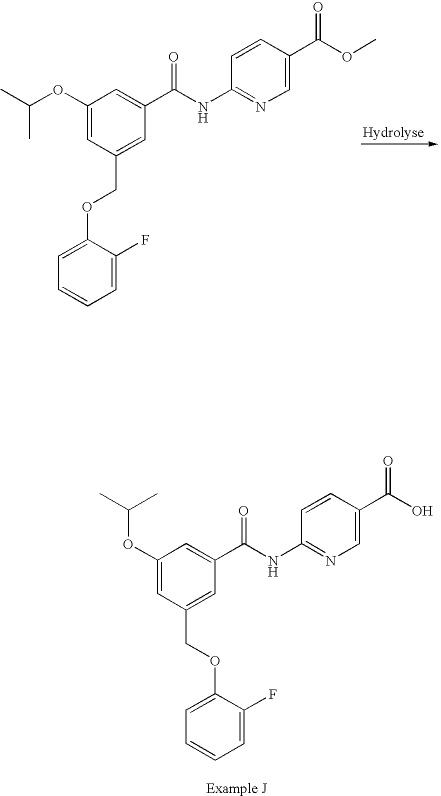

2M NaOH (0.55 ml, 1.1 mM) was added to methyl 2-[3-isopropyloxy-5-(2-fluorophenoxy) methyl benzoyl]amino-5-pyridine carboxylate (0.16 g, 0.36 mM) in THF (10 ml)/water (10 ml) at ambient temperature. After 4 hrs the reaction mixture was neutralised to pH=4-5 with 2M HCl, concentrated, filtered, washed with water, and dried under high-vacuum to give the title compound as a colourless solid (0.15 g, 98%);

¹H NMR δ (d₆-DMSO): 1.28 (d, 6H), 4.74 (m, 1H), 5.20 (s, 2H), 6.87-6.97 (m, 1H), 7.10 (m, 1H), 7.16-7.26 (m, 3H), 7.54 (s, 1H), 7.66 (s, 1H), 8.28 (s, 2H), 8.84 (s, 1H), 11.78 (bs, 1H).

The requisite intermediate methyl ester was prepared as follows:

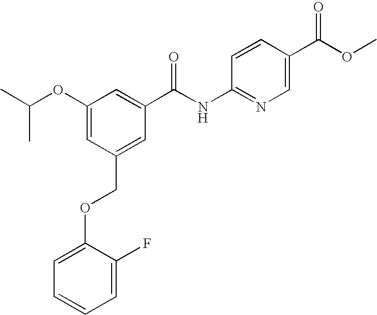

Oxalyl chloride (0.20 ml, 2.35 mM) was added to 3-isopropyloxy-5-(2-fluorophenoxy) methyl benzoic acid (0.20 g, 0.66 mM) in dichloromethane (10 ml) containing DMF (2 drops) under an argon atmosphere at room temperature. After 2 hrs the reaction mixture was concentrated in vacuo. The acid chloride and methyl 2-amino-pyridine-5-carboxylate (0.1 g, 0.66 mM) were dissolved in pyridine (5 ml) and stirred under argon overnight. The reaction mixture was concentrated and triturated with MeOH to give the title compound as a colourless solid (0.19 g, 66%); ¹H NMR δ (d₆-DMSO): 1.29 (d, 6H), 3.85 (s, 3H), 4.74 (m, 1H), 5.18 (s, 2H), 6.93 (m, 1H), 7.10 (m, 1H), 7.16-7.26 (m, 3H), 7.53 (s, 1H), 7.66 (s, 1H), 8.32 (s, 2H), 8.89 (s, 1H), 11.21 (bs, 1H).

The requisite 3-isopropyloxy-5-(2-fluorophenoxy) methyl benzoic acid startting material was prepared as follows:

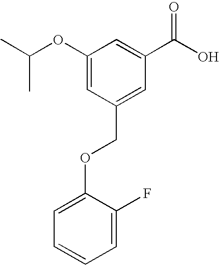

2M NaOH (4.2 ml, 8.4 mM) was added to a solution of methyl 3-isopropyloxy-5-(2-fluorophenoxy) methyl benzoate (0.67 g, 2.1 mM) in MeOH (20 ml)/THF (4 ml). After 5 hrs, the reaction mixture was concentrated, acidified to pH=1-2 (2M HCl), filtered and dried under high vacuum to give the title compound as a colourless solid (0.62 g, 97%);

¹H NMR δ (d₆-DMSO): 1.25 (d, 6H), 4.61 (m, 1H), 5.18 (s, 2H), 6.92 (m, 1H), 7.05-7.24 (m, 4H), 7.34 (s, 1H), 7.54 (s, 1H).

The requisite methyl 3-isopropyloxy-5-(2-fluorophenoxy) methyl benzoate starting material was prepared as follows:

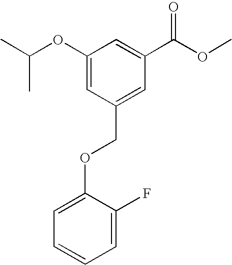

DIAD (0.74 ml, 3.7 mM) was added to methyl 3-isopropyloxy-5-hydroxymethyl benzoate (0.56 g, 2.5 mM), triphenylphosphine (0.98 g, 3.7 mM) and 2-fluorophenol (0.24 ml, 2.7 mM) in DCM (40 ml) under argon at ambient temperature. After 10 mins the reaction mixture was concentrated and purified on silica gel (10-15% EtOAc/iso-hexane) to give the title compound as a pale yellow oil, which solidified under high-vacuum (0.71 g, 90%); ¹H NMR δ (d₆-DMSO): 1.26 (d, 6H), 3.82 (s, 3H), 4.64 (m, 1H), 5.21 (s, 2H), 6.92 (m, 1H), 7.09 (m, 1H), 7.16-7.26 (m, 3H), 7.35 (s, 1H), 7.58 (s, 1H).

The requisite methyl 3-isopropyloxy-5-hydroxymethyl benzoate starting material was prepared as follows:

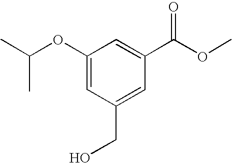

Mono-methyl-5-isopropyloxy-isophthalate (5.15 g, 21.6 mM) was dissolved in THF (180 ml), cooled to 2° C. and borane. THF complex (72 ml of 1.5M solution in THF, 0.11 mM) added dropwise over 15 mins, maintaining an internal temperature of <5° C. After mins the reaction mixture was warmed to ambient temperature, stirred for 3 hrs before cooling (ice bath) and quenching with pieces of ice. When no further reaction observed brine (150 ml)/diethyl ether (150 ml) added. The organic layer was removed, aqueous extracted with additional diethyl ether (1×100 ml), combined organics washed with brine (1×100 ml), dried (MgSO₄), filtered and concentrated. Purified on silica gel (20-25% EtOAc/isohexane) to give the title compound as a colourless solid (3.57 g, 74%); ¹H NMR δ (d₆-DMSO): 1.26 (d, 6H), 3.82 (s, 3H), 4.50 (d, 2H), 4.63 (m, 1H), 5.26 (t, 1H (—OH)), 7.10 (s, 1H), 7.25 (s, 1H), 7.47 (s, 1H).

The requisite mono-methyl-5-isopropyloxy-isophthalate starting material was prepared as follows:

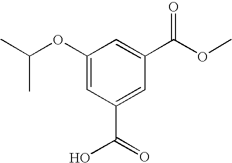

2M NaOH (1.03 g, 25.9 mM) in MeOH (9 ml) was added to a solution of dimethyl 5-isopropyloxy-isophthalate (5.68 g, 22.5 mM) in acetone (45 ml) and stirred at ambient temperature overnight. The reaction mixture was concentrated, acidified (2M HCl) to pH=1-2, filtered, washed with water and dried under high vacuum to give a colourless solid (5.25 g, 98%) (contains 15-20% diacid); MS (M−H⁺)⁻ 237.

The requisite dimethyl 5-isopropyloxy-isophthalate starting material was prepared as follows:

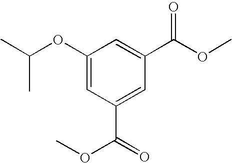

Dimethyl-5-hydroxy-isophthalate (5.2 g, 24.6 mM), potassium carbonate (4.07 g, 29.5 mM), potassium iodide (0.82 g, 4.9 mM) and 2-bromopropane (2.4 ml, 25.8 mM) in DMF (50 ml) were heated at 90° C. for 3 hrs, after which time additional 2-bromopropane (2.4 ml), potassium carbonate (2.2 g) were added, and heating continued for a further 4 hrs. The reaction mixture was then cooled to room temperature and concentrated. EtOAc (150 ml) was added then washed with water, brine, dried (MgSO₄), filtered and concentrated to give a pale yellow oil which solidified on standing (6.0 g, 97%); MS (MH⁺) 253.

### EXAMPLE K

### 2-[3-isopropyloxy-5-{(2-fluorobenzylamino)methyl}benzoylamino]-5-pyridine Carboxylic Acid (Route 11)

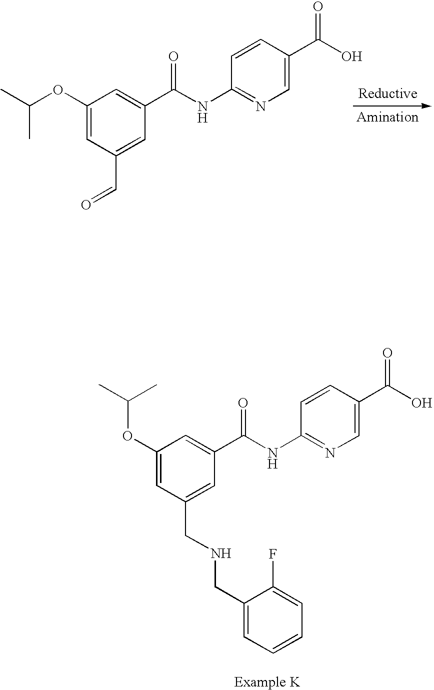

2-(3-isopropyloxy-5-carboxy-benzoyl) amino-5-pyridine carboxylic acid (0.10 g, 0.30 mM), 4A molecular sieves (0.3 g) and 2-fluorobenzylamine were stirred in MeOH at ambient temperature for 2 hrs then sodium cyanoborohydride (0.023 g, 0.36 mM) added. After a further 2 hrs the reaction mixture was filtered, residue washed with MeOH and the filtrate concentrated in vacuo. Water was added, then acidified with 2M HCl to precipitate a colourless solid which was filtered, washed with water and dried under high-vacuum to give the title compound as a light brown solid (00.10 g, 76%); ¹H NMR δ (d₆-DMSO): ¹H NMR δ (d₆-DMSO): 1.29 (d, 6H), 4.13 (d, 2H), 4.74 (m, 1H), 7.20-7.30 (m, 3H), 7.43 (m, 1H), 7.58 (m, 2H), 7.68 (s, 1H), 8.28 (s, 2H), 8.87 (s, 1H), 11.10 (bs, 1H).

The requisite aldehyde intermediate was prepared as follows:

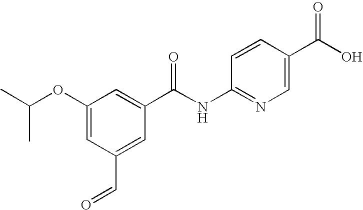

To 2-(3-isopropoxy-5-hydroxymethyl-benzoyl) amino-5-pyridine carboxylic acid (0.33 g, 1.0 mM) in THF (20 ml) under argon, Dess-Martin periodinane (0.46 g, 1.1 mM) was added in one portion. After 45 mins satd. potassium carbonate (20 ml) was added and the THF removed in vacuo. Residue was stirred with 2.0M Na₂S₂O₃ (3.5 ml, 7 mM) for 35 mins then acidified cautiously to pH=1 with 2M HCl. Resulting suspension was filtered, washed with water, diethyl ether, DCM and dried under high-vacuum to give 2-(3-isopropyloxy-5-carboxy-benzoyl) amino-5-pyridine carboxylic acid as a pale yellow solid (0.3 g, 93%); ¹H NMR δ (d₆-DMSO): 1.32 (d, 6H), 4.82 (m, 1H), 7.58 (m, 1H), 7.84 (m, 1H), 8.11 (s, 1H), 8.29 (s, 2H), 8.87 (s, 1H), 10.02 (s, 1H), 11.34 (bs, 1H).

The requisite intermediate methyl alcohol (Example L) was prepared as described below.

### EXAMPLE L

2-(3-isopropoxy-5-hydroxymethyl-benzoylamino)-5-pyridine carboxylic acid (Route 12)

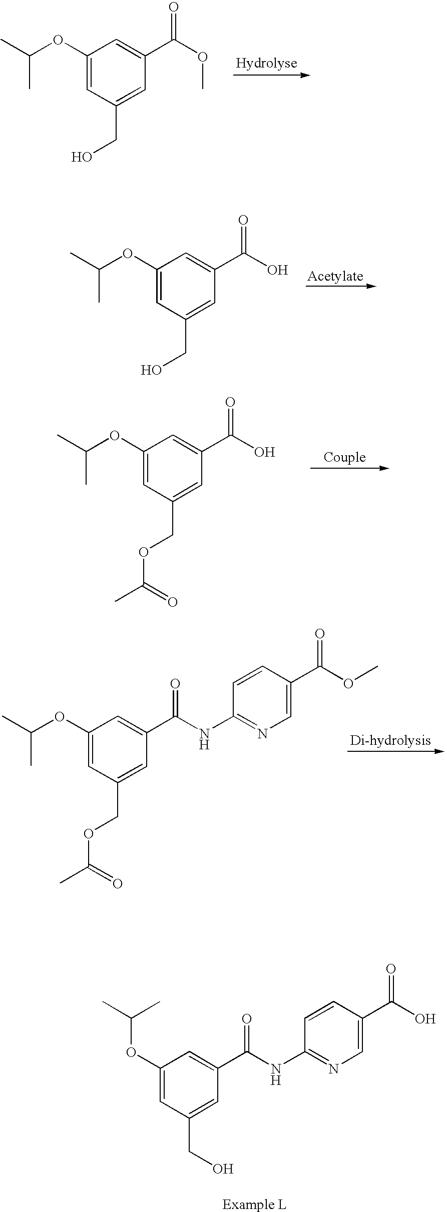

The title compound was prepared using standard hydrolysis conditions (2M NaOH/THF/MeOH) starting from methyl 2-(3-isopropoxy-5-acetoxymethyl) benzoylamino-5-pyridine carboxylate (0.85 g, 2.2 mM), giving the title compound as a colourless solid (0.13 g, 92%); ¹H NMR δ (d₆-DMSO): 1.28 (d, 6H), 4.50 (s, 2H), 4.72 (m, 1H), 7.06 (s, 1H), 7.42 (s, 1H), 7.53 (s, 1H), 8.29 (s, 2H), 8.87 (s, 1H), 11.09 (bs, 1H).

The requisite diester intermediate was prepared as follows:

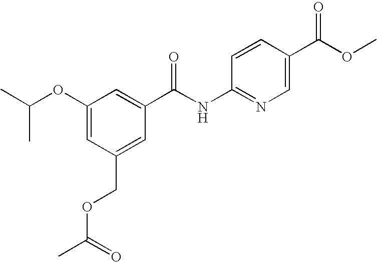

Standard amide coupling (oxalyl chloride/DMF in dichlorormethane) between 3-isopropoxy-5-acetoxymethyl benzoic acid and methyl 2-aminopyridine-5-carboxylate gave methyl 2-(3-isopropoxy-5-acetoxymethyl) benzoylamino-5-pyridine carboxylate as a colourless solid (1.0 g, 72%); ¹H NMR δ (d₆-DMSO): 1.29 (d, 6H), 2.08 (s, 3H), 3.85 (s, 3H), 4.74 (m, 1H), 5.07 (s, 2H), 7.10 (s, 1H), 7.53 (s, 1H), 7.55 (s, 1H), 8.31 (s, 2H), 8.89 (s, 1H), 11.19 (bs, 11H).

The requisite acetoxymethyl benzoic acid intermediate was prepared as follows:

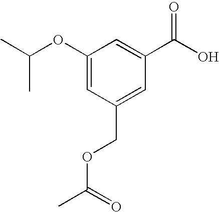

3-isopropoxy-5-hydroxymethyl benzoic acid (0.77 g, 3.7 mM) was dissolved in DCM (20 ml), pyridine (1.18 ml, 14.6 mM) added, cooled (ice bath) then acetyl chloride (0.55 ml, 7.7 mM) added. The reaction mixture was warmed to ambient temperature, after 2 hrs water (20 ml) was added and stirred overnight. After which organic layer washed with 0.05M HCl (1×20 ml), dried (MgSO₄), filtered and concentrated to give 3-isopropoxy-5-hydroxymethyl benzoic acid as a pale yellow solid (1.12 g, 93%); ¹H NMR δ (d₆-DMSO): 1.25 (d, 6H), 2.06 (s, 3H), 4.64 (m, 1H), 5.06 (s, 2H), 7.12 (s, 1H), 7.31 (s, 1H), 7.46 (s, 1H).

The requisite hydroxymethyl methyl benzoic acid intermediate was prepared as follows:

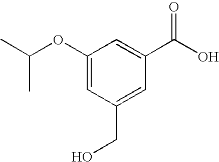

Standard ester hydrolysis (2M NaOH/THF/MeOH) of methyl 3-isopropyloxy-5-hydroxymethyl benzoate (described in Example J) (1.12 g, 5.0 mM) gave 3-isopropoxy-5-hydroxymethyl benzoic acid as a colourless solid (0.98 g, 94%); ¹H NMR δ (d₆-DMSO): 1.25 (d, 6H), 4.47 (s, 2H), 4.60 (m, 1H), 5.23 (bs, 1H), 7.06 (s, 1H), 7.24 (s, 1H), 7.45 (s, 1H).

### EXAMPLE M

2-{3-isopropyloxy-5-[2-(2-pyridyl)ethenyl]benzoylamino}-5-pyridine carboxylic acid (Route 13)

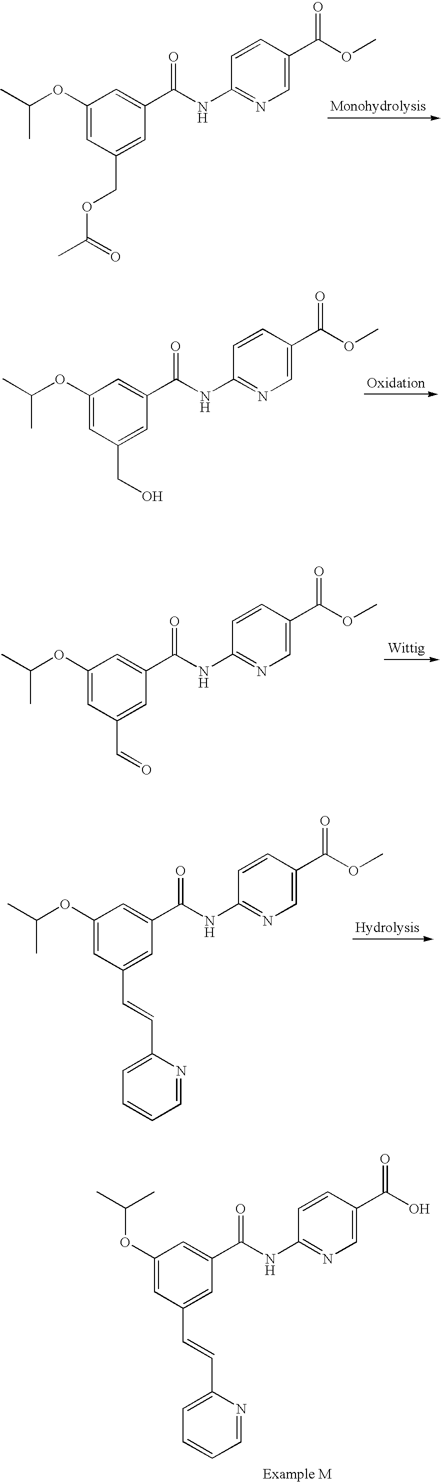

Standard ester hydrolysis (2M NaOH/THF) of methyl 2-{3-isopropyloxy-5-[2-(2-pyridyl)ethenyl]benzoyl}amino-5-pyridine carboxylate gave the title compound as a pale yellow solid (0.024 g, 34%); ¹H NMR δ (d₆-DMSO): ¹H NMR δ (d₆-DMSO): 1.32 (d, 6H), 4.82 (m, 1H), 7.40 (s, 1H), 7.49-7.58 (m, 1H), 7.61 (d, 1H), 7.62 (m, 1H), 7.72 (m, 1H), 7.91 (s, 1H), 8.03 (d, 1H), 8.13 (d, 1H), 8.32 (m, 2H), 8.74 (m, 1H), 8.89 (m, 1H), 11.28 (bs, 1H).

The requisite methyl ester intermediate was prepared as follows:

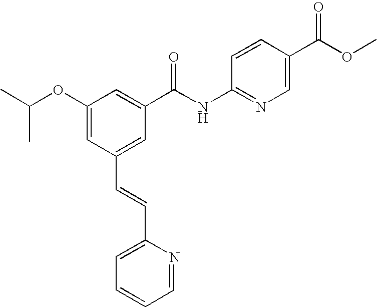

Triphenyl(2-pyridylmethyl)phosphonium chloride hydrochloride (0.12 g, 0.28 mM) was suspended in THF (10 ml) and potassium tert-butoxide (1.0M in THF) (0.55 ml, 0.55 mM) added under an argon atmosphere. After 15 mins the solution was transferred via syringe into a cooled (ice bath) solution of methyl 2-(3-isopropyloxy-5-carboxy-benzoyl) amino-5-pyridine carboxylate (0.079 g, 0.23 mM) in THF (10 ml) under an argon atmosphere. The reaction mixture was allowed to warm to room temperature overnight then water added, concentrated in vacuo, extracted with ethyl acetate, organic extracts dried (MgSO₄), filtered and concentrated in vacuo. Purification on silica gel (10 g bond elute, loaded in DCM, eluting with 15% to 30% EtOAc/iso-hexane) gave methyl 2-{3-isopropyloxy-5-[2-(2-pyridyl)ethenyl]benzoyl}amino-5-pyridine carboxylate as a colourless film (0.07 g, 73%); MH⁺=418

The requisite aldehyde intermediate was prepared as follows:

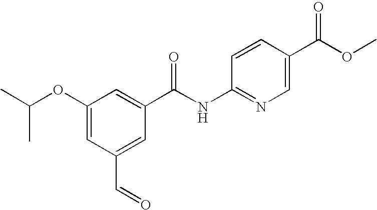

Standard Dess-Martin periodinane oxidation (described in Example K) of methyl 2-(3-isopropyloxy-5-hydroxymethyl benzoyl) amino-5-pyridine carboxylate (0.37 g, 1.1 mM) gave methyl 2-(3-isopropyloxy-5-carboxy-benzoyl) amino-5-pyridine carboxylate as a colourless solid (0.32 g, 87%); ¹H NMR δ (d₆-DMSO): 1.32 (d, 6H), 3.85 (s, 3H), 4.82 (m, 1H), 7.58 (m, 1H), 7.84 (m, 1H), 8.08 (s, 1H), 8.32 (s, 2H), 8.89 (s, 1H), 10.02 (s, 1H), 11.40 (bs, 1H).

The requisite intermediate methyl alcohol was prepared as follows:

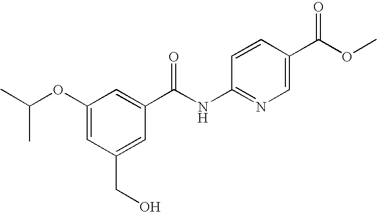

Potassium carbonate (0.197 g, 1.42 mM) was added to a solution of methyl 2-(3-isopropyloxy-5-acetoxymethyl)benzoyl amino-5-pyridine carboxylate (0.55 g, 1.42 mM) in MeOH (25 ml)/water (2.5 ml). After stirring at ambient temperature for 2 hrs the reaction mixture was acidified with 2M HCl to precipitate a solid, which was collected by filtration and dried under high vacuum to give the title compound as a colourless solid (0.40 g, 82%); ¹H NMR δ (d₆-DMSO): 1.3 (d, 6H), 3.85 (s, 3H), 4.55 (d, 2H), 4.75 (hept, 1H), 5.25 (t, 1H), 7.05 (s, 1H), 7.45 (s, 1H), 7.55 (s, 1H), 8.35 (d, 2H), 8.9(d, 1H), 11.1 (bs, 1H); m/z 345 (MH)⁺, 343 (M−H)⁻

The requisite methyl 2-(3-isopropyloxy-5-acetoxymethyl)benzoyl amino-5-pyridine carboxylate was prepared as described in Example L.

### EXAMPLE N

2-{3-isopropyloxy-5-[(N-methyl) 4-toluenesulfonylaminomethyl]benzoylamino}-5-pyridine carboxylic acid (Route 14)

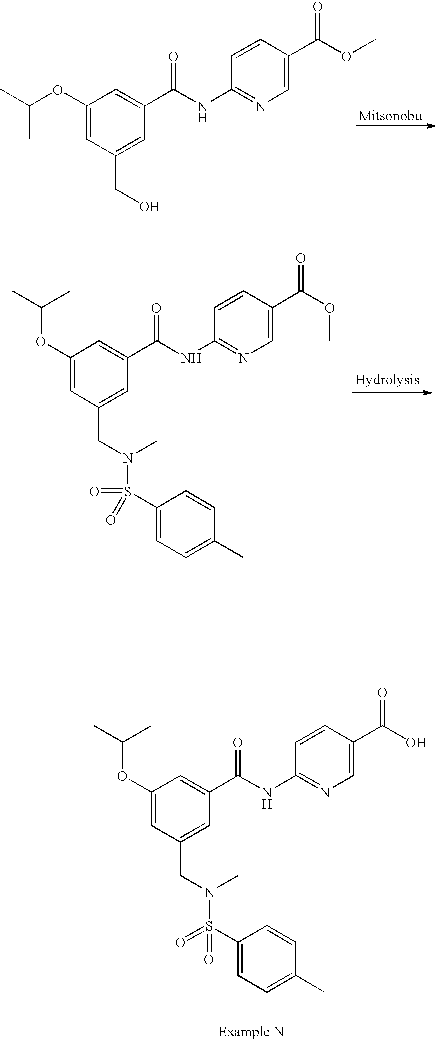

Standard ester hydrolysis (2M NaOH/THF), as described in Example A, of methyl 2-{3-isopropyloxy-5-[(N-methyl) 4-toluenesulfonylaminomethyl]benzoyl}amino-5-pyridine carboxylate gave the title compound as a pale yellow solid, ¹H NMR δ (d₆-DMSO): 1.23 (d, 6H), 2.40 (s, 3H), 2.58 (s, 3H), 4.13 (s, 2H), 4.62-4.72 (m, 1H), 7.70 (s, 1H), 7.41-7.52 (m, 4H), 7.73 (d, 2H), 8.31 (s, 2H), 8.84 (s, 1H), 11.16 (s, 1H) m/z 498 (MH)⁺, 496 (M−H)⁻.

The requisite methyl ester starting material was prepared as follows:

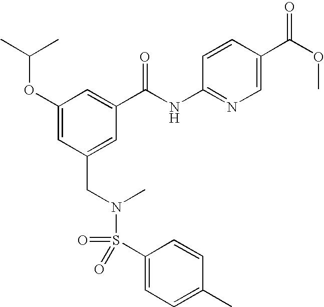

Methyl 2-(3-isopropyloxy-5-hydroxymethyl benzoyl)amino-5-pyridine carboxylate (100 mg, 0.29 mM), tributylphosphine (88 mg, 0.44 mM) and N-methyl-p-toluenesulfonamide (82 mg, 0.44 mM) were successively dissolved in anhydrous toluene, with stirring under an argon atmosphere at 0° C. Solid 1,1′-(azodicarbonyl)dipiperidine (ADDP) (111 mg, 0.44 mM) was then added to the solution. After 10 minutes, the reaction mixture was brought to room temperature and stirring continued for 24 hrs. Hexane was added to the reaction mixture and dihydro-ADDP separated out and was removed by filtration. The product was purified on silica gel (gradient 0-100% EtOAc/iso-hexane) to yield the product as a colourless solid (51 mg, 0.1 mM, 34%);

¹H NMR δ (d₆-DMSO): 1.25 (d, 6H), 2.4 (s, 3H), 2.59 (s, 3H), 3.83 (s, 3H), 4.14 (s, 2H), 4.62-4.72 (m, 1H), 7.00 (s, 1H), 7.42 (d, 2H), 7.48 (s, 2H), 7.72 (d, 2H), 8.34 (s, 2H), 8.90 (s, 1H), 11.21 (bs, 1H).

The requisite benzyl alcohol starting material was prepared as described in Example M.

### EXAMPLE O

2-[3-(2-fluorobenzyloxy)-5-(5-methylisoxazol-3-ylmethoxymethyl)-benzoylamino]-5-pyridine carboxylic acid (Route 15)

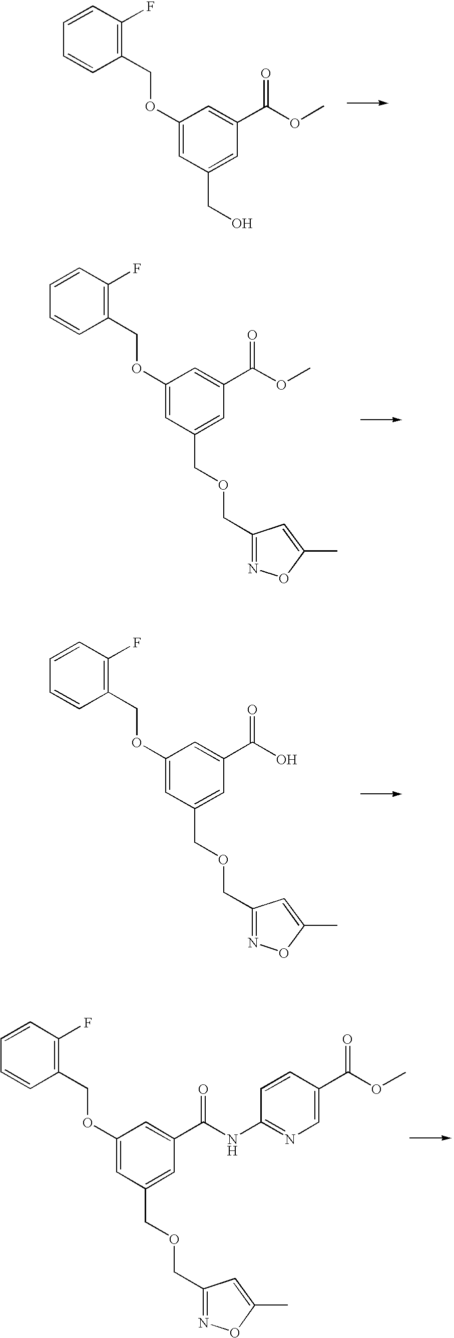

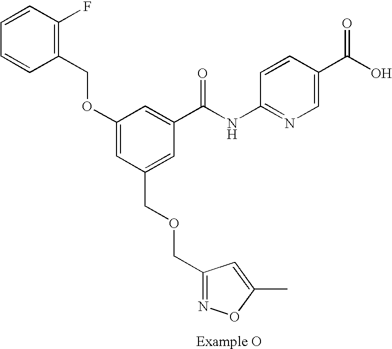

Standard ester hydrolysis (2M NaOH/THF), as described in Example A, of methyl 2-[3-(2-fluorobenzyloxy)-5-(5-methyl isoxazol-3-yl methoxy) methyl benzoyl]aminopyridine-5-carboxylate gave the title compound as a colourless solid, ¹H NMR δ (300 MHz, d₆-DMSO): 2.40 (s, 3H); 4.58 (s, 4H), 5.22 (s, 2H); 6.26 (s, 1H); 7.21-7.30 (m, 3H); 7.38-7.45 (m, 1H); 7.55-7.60 (ap d, 1H); 7.60 (s, 1H); 7.64 (s, 1H); 8.32 (s, 2H); 8.86 (s, 1H); 11.16 (br s, 1H); m/z 492 (M+H)⁺, 490 (M−H)⁻

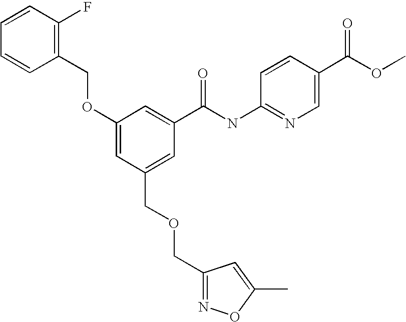

The requisite methyl ester starting material was prepared by a standard oxalyl chloride coupling, starting from 3-(2-fluorobenzyloxy)-5-(5-methyl isoxazol-3-yl methoxy) methyl benzoic acid, as described in Example A (Route 1), to give methyl 2-[3-(2-fluorobenzyloxy)-5-(5-methyl isoxazol-3-yl methoxy) methyl benzoyl]aminopyridine-5-carboxylate, ¹H NMR δ (d₆-DMSO): 2.40 (s, 3H); 3.86 (s, 3H); 4.58 (ap d, 4H); 5.22 (s, 2H); 6.27 (s, 1H), 7.20-7.30 (m, 3H); 7.39-7.46 (m, 1H); 7.59 (d, 1H); 7.61 (s, 2H); 7.68 (s, 1H); 8.37 (s, 2H); 8.91 (s, 1H); 11.22 (br s, 1H); m/z 506 (M+H)⁺.

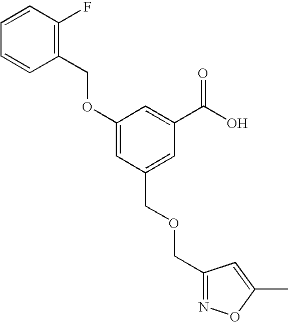

The requisite 3-(2-fluorobenzyloxy)-5-(5-methyl isoxazol-3-yl methoxy) methyl benzoic acid starting material was prepared by a standard hydrolysis of methyl 3-(2-fluorobenzyloxy)-5-(5-methyl isoxazol-3-yl methoxy) methyl benzoate as described in the generic Alkylation Methods, and in the manner outlined in Examples C and E; ¹H NMR δ (d₆-DMSO): 2.40 (s, 3H); 4.54 (s, 2H); 4.57 (s, 2H); 5.20 (s, 2H); 6.24 (s, 1H); 7.18-7.28 (m, 3H); 7.39-7.47 (m, 2H); 7.50-7.60 (m, 2H); m/z 370 (M−H)⁻.

The requisite methyl 3-(2-fluorobenzyloxy)-5-(5-methyl isoxazol-3-yl methoxy) methyl benzoate starting material was prepared as follows:

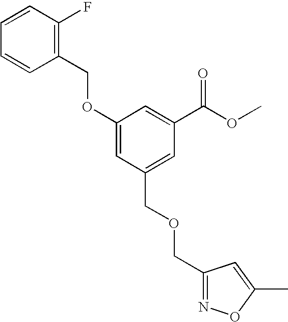

Sodium hydride (60% dispersion in oil, 83 mg, 2.07 mM) was added to a solution of methyl 3-(2-fluorobenzyloxy)-5-hydroxymethyl benzoate (400 mg, 1.38 mM) in THF (10 ml) at 0° C. The reaction mixture was allowed to warm to ambient temperature before adding 3-chloromethyl-5-methylisoxazole (272 mg, 2.07 mM). The reaction mixture was stirred at room temperature for 24 hrs. The reaction was quenched with water (5 ml), then diluted with ethyl acetate (10 ml). The organic phase was separated and dried over magnesium sulfate and concentrated in vacuo to a yellow oil (462 mg, 1.2 mM, 87%) which was used without further purification; ¹H NMR δ (d₆-DMSO): 2.39 (s, 3H); 3.82 (s, 3H); 4.56 (s, 2H); 4.58 (s, 2H); 5.20 (s, 2H); 6.24 (s, 1H); 7.18-7.28 (m, 3H); 7.38-7.42 (t, 1H); 7.48 (s, 1H); 7.50-7.58 (m, 2H); m/z 386 (M+H)⁺.

The requisite methyl 3-(2-fluorobenzyloxy)-5-hydroxymethyl benzoate starting material was prepared as described in footnote (f).

### EXAMPLE P

2-[3-isopropyloxy-5-(2-fluorophenylsulfonylmethyl)benzoylamino]-5-pyridine carboxylic acid (Route 16)

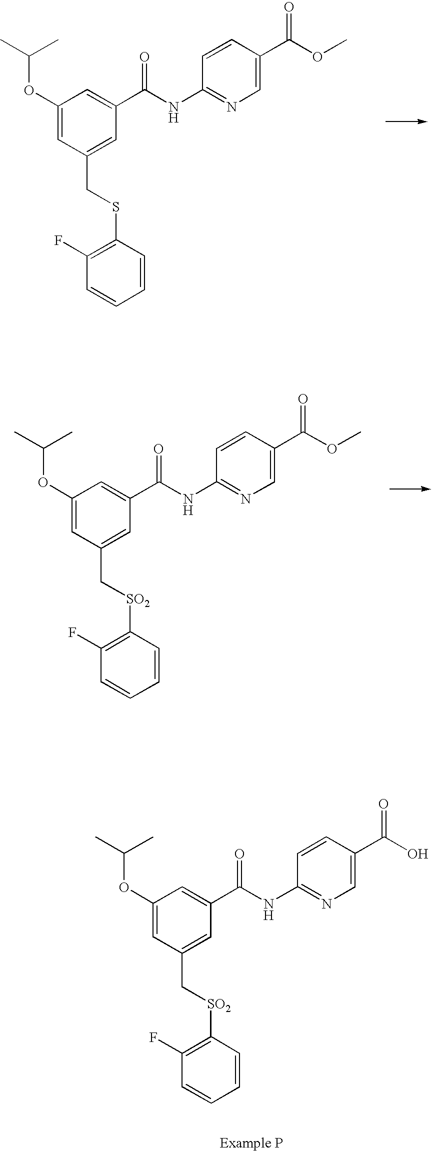

Standard ester hydrolysis (2M NaOH/THF), as described in Example A, of methyl 2-[3-isopropyloxy-5-(2-fluorophenylsulfonyl) methyl benzoyl]aminopyridine-5-carboxylate gave the title compound as a pale yellow solid, ¹H NMR δ (300 MHz, d₆-DMSO): 1.12 (d, 6H); 4.58-4.66 (m, 1H); 4.79 (s, 2H); 6.98 (s, 1H); 7.30-7.41 (m, 2H); 7.43 (s, 1H); 7.48-7.63 (m, 2H); 7.72-7.81 (m, 1H); 8.30 (s, 2H); 8.86 (S, 1H); 11.08 (br s, 1H); m/z 473 (M+H)⁺, 471

(M−H)⁻ 0.4

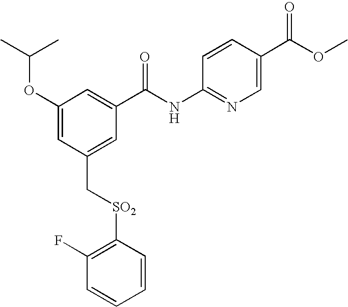

To a stirred solution of methyl 2-[3-isopropyloxy-5-(2-fluorophenylsulfanyl) methyl benzoyl]aminopyridine-5-carboxylate (300 mg, 0.66 mM) in glacial acetic acid (10 ml) was added a solution of potassium permanganate (151 mg, 0.96 mM) in water (8 ml). The resulting brown solution was allowed to stir at room temperature for 2 hrs. Sodium sulfite solid was added until the reaction mixture became clear and colourless. Ethyl acetate was added and the organic phase was washed with a saturated solution of sodium hydrogen carbonate (4×50 ml). The organic phase was separated, dried over magnesium sulfate and concentrated in vacuo to give a yellow oil. This was purified on silica gel (gradient 0-100% EtOAc/iso-hexane) to yield methyl 2-[3-isopropyloxy-5-(2-fluorophenylsulfonyl) methyl benzoyl]aminopyridine-5-carboxylate as a colourless solid (70 mg, 0.14 mM, 21%); m/z 487 (M+H)⁺.

The requisite sulfide starting material was prepared as described in Example J (Route 10).

### EXAMPLE Q

2-[3-isobutyloxy-5-(3-thienyl) benzoylmino]-5-pyridine carboxylic acid (Route 17)

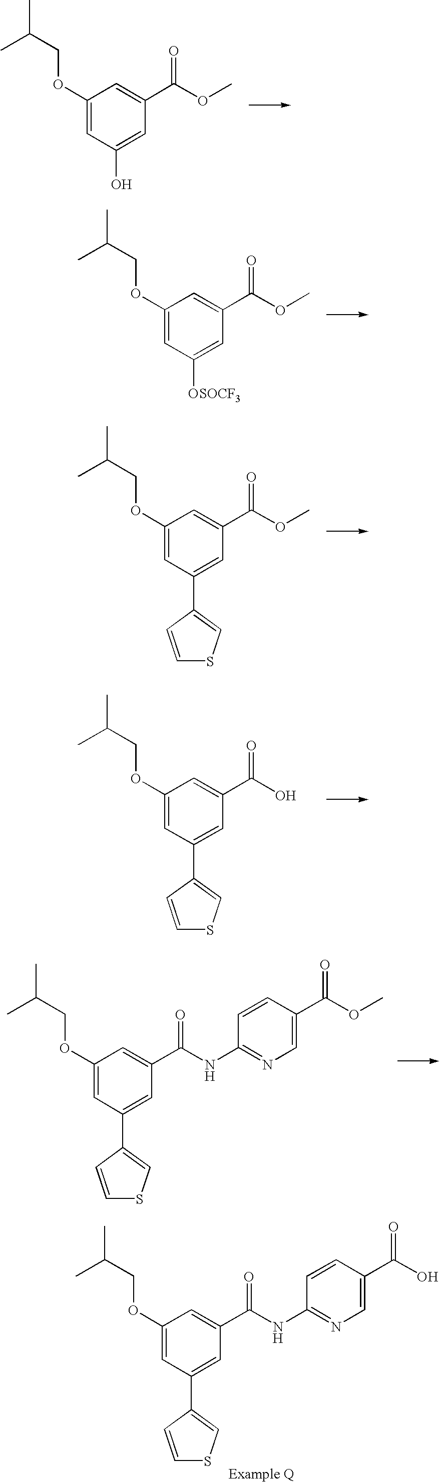

Standard ester hydrolysis (2M NaOH/THF), as described in Example A, of methyl 2-[3-isobutyloxy-5-(3-thienyl) benzoyl]aminopyridine-5-carboxylate gave the title compound as a pale yellow solid, m/z 397 (M+H)⁺ 395 (M−H)⁻; LC-MS: retention time 2.84 mins, 93% purity.

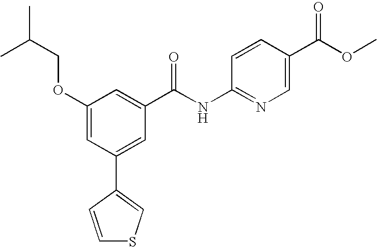

The requisite methyl ester starting material was prepared by a standard oxalyl chloride coupling, starting from 2-[3-isobutyloxy-5-(3-thienyl) benzoic acid, as described in Example A (Route 1), to give methyl 2-[3-isobutyloxy-5-(3-thienyl) benzoyl]aminopyridine-5-carboxylate, ¹H NMR δ (d₆-DMSO): 1.01 (d, 6H), 2.03 (m, 1H), 3.85 (d, 2H), 7.33 (m, 1H), 7.47 (m, 2H), 7.63 (m, 1H), 7.68 (m, 1H), 7.98 (m, 1H), 8.47 (m, 2H), 8.92 (s, 1H), 11.27 (br 5, 1H); m/z 411 (M+H)⁺.

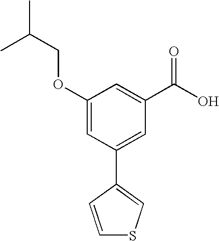

The requisite 2-[3-isobutyloxy-5-(3-thienyl) benzoic acid starting material was prepared by a standard hydrolysis of methyl 2-[3-isobutyloxy-5-(3-thienyl) benzoate as dscribed in the generic Alkylation Methods, and in the manner outlined in Examples C and E; ¹H NMR δ (d₆-DMSO): 0.99 (d, 6H), 2.03 (m, 1H), 3.84 (d, 2H), 7.32 (m, 1H), 7.46 (m, 1H), 7.57 (m, 1H), 7.62 (m, 1H), 7.76 (s, 1H), 7.97 (m, 1H).

The requisite methyl 2-[3-isobutyloxy-5-(3-thienyl) benzoate starting material was prepared as follows:

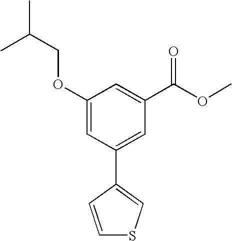

Thiophene-3-boronic acid (0.134 g, 1.0 mM), methyl 3-isobutyloxy-5-(trifluoromethanesulfonyloxy) benzoate (“triflate”) (0.34 g, 0.95 mM), and bis(triphenylphosphine)palladium dichloride (0.067 g, 0.09 mM) were suspended in a mixture of toluene and satd. aq.NaHCO₃ (5 ml of each) and heated at 100° C. under an argon atmosphere. After 3 hrs the reaction mixture was cooled, satd. Aq. NH₄Cl added, the organic layer separated and the aqueous layer then extracted with EtOAc (2×10 ml). The combined organics were dried (MgSO₄), filtered, concentrated in vacuo to yield a black oil.

Purification on silica gel (iso-hexane then 2% EtOAc/iso-hexane) gave methyl 3-isobutyloxy-5-(3-thienyl) benzoate as a colourless oil (0.205 g, 7-4%); ¹H NMR δ (d₆-DMSO):0.99 (d, 6H), 2.03 (m, 1H), 3.84 (m, 5H), 7.33 (m, 1H), 7.51 (m, 1H), 7.58 (m, 1H), 7.63 (m, 1H), 7.79 (s, 1H), 7.99 (m, 1H).

The requisite triflate starting material was prepared as follows:

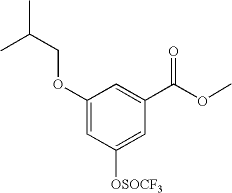

Trifluoromethanesulphonic anhydride (2.3 ml, 13.9 mM) was added dropwise over 2 mins to a solution of the methyl 3-isobutyloxy-5-hydroxy benzoate (2.97 g, 13.2 mM) in DCM (80 ml) at −78° C. under an argon atmosphere. After 1 hr the solution was warmed to ambient temperature, stirred for 30 mins then sat.aq. NaHCO₃ added. The organic layer was separated, dried (MgSO₄), filtered and concentrated in vacuo to give a yellow oil. Purification on silica gel (5% EtOAc/iso-hexane) gave methyl 3-isobutyloxy-5-(trifluoromethanesulfonyloxy) benzoate as a colourless oil (2.64 g, 56%); ¹H NMR δ (d₆-DMSO):0.97 (d, 6H), 2.02 (m, 1H), 3.85 (m, 5H), 7.42 (m, 1H), 7.47 (m, 1H), 7.53 (m, 1H).

The requisite methyl 3-isobutyloxy-5-hydroxy benzoate starting material was prepared as described in generic Alkylation Method B; ¹H NMR δ (d₆-DMSO): 0.98 (d, 6H); 1.90-2.03 (m, 1H); 3.70 (d, 2H); 3.79 (s, 3H); 6.57 (t, 1H); 6.88 (s, 1H); 6.94 (s, 1H); 9.78 (s, 1H); m/z 225 (M+H)⁺, 223 (M−H)⁻.

### EXAMPLE R

2-{3-[2-(thien-2-yl)-ethoxy]-5-(4-chlorophenoxy)benzoylamino}-5-pyridine Carboxylic Acid (Route 18)

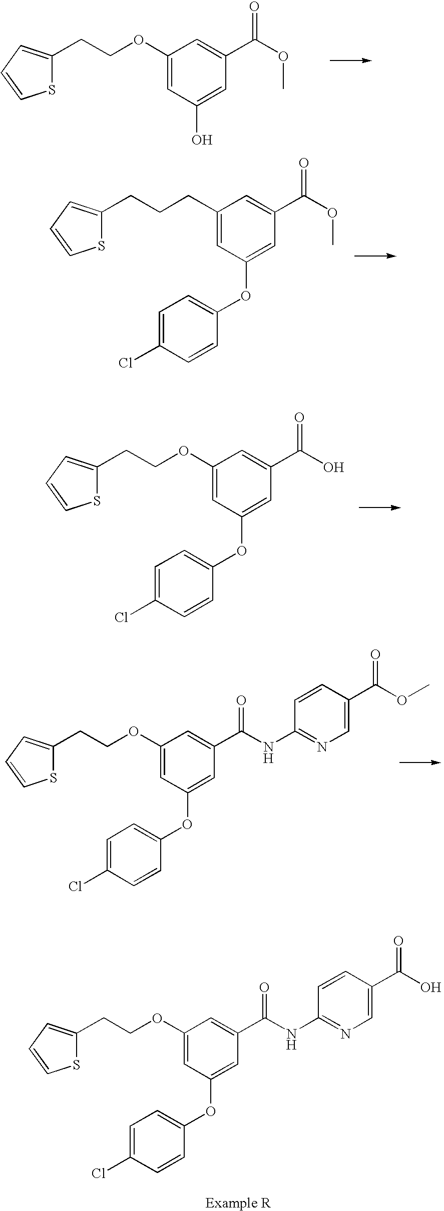

1M NaOH (0.263 ml, 0.26 mM) was added to a solution of methyl 2-{3-[2-(thien-2-yl)-ethoxy]-5-(4-chlorophenoxy)}benzoyl amino-5-pyridine carboxylate (44.7 mg, 0.088 mM) in THF (1 ml)/methanol (50 μl). After 17 hr the reaction mixture was neutralised with 1M citric acid, then concentrated in vacuo. The pH was adjusted to 3-4 with 1M citric acid, filtered, dried under high vacuum to give the title compound as a pale yellow solid (16.1 mg, 37%); ¹H NMR δ (d₆-DMSO): 3.27 (2H, t), 4.30 (2H, t), 6.85. (1H, m), 6.98 (2H, m), 7.10 (2H, m), 7.22 (1H, m), 7.33 (1H, m), 7.46 (3H, m), 8.28 (2H, m), 8.88 (1H, s), 11.19 (1H, br s).

The starting methyl ester intermediate was prepared as follows:

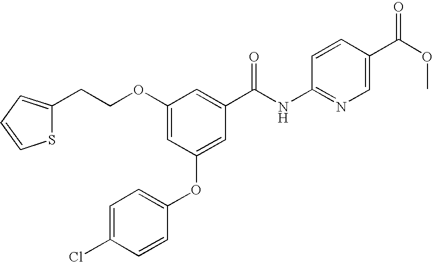

A solution of 3-(4-chlorophenoxy)-5-(2-thiophen-2-yl)ethoxy benzoic acid (67.5 mg, 0.18 mM) and the methyl-6-amino-nicotinate (35 mg, 0.22 mM) in anhydrous pyridine (1 ml), was treated with phosphorous oxychloride (24 μl, 2.3 mM) The mixture was left to stir at room temperature under argon for 18 hours. The solvent was removed in vacuo and the residues treated with H₂O (5 ml) and acidified to pH=3-4 with 1M citric acid. The aqueous was extracted with EtOAc (2×20 ml) and the organics washed with brine (10 ml), dried (MgSO₄) and evaporated in vacuo to give a brown oil which was purified on silica gel (10% to 50% EtOAc in isohexane) to afford methyl 2-{3-[2-(thien-2-yl)-ethoxy]-5-(4-chlorophenoxy)}benzoyl amino-5-pyridine carboxylate as a clear colourless oil (44.7 mg, 49%). ¹H NMR δ (CDCl₃): 3.32 (2H, t), 3.94 (3H, s), 4.22 (2H, t), 6.77 (1H, s), 6.91-7.00 (3H, br m), 7.09 (1H, s), 7.19 (2H, m), 7.34 (2H, m), 8.34 (1H, m), 8.42 (1H, m), 8.63 (1H, s), 8.92 (1H, s); m/z 511 (M+H)⁺, 509 (M+H)⁺.

The requisite 3-(4-chlorophenoxy)-5-(2-thiophen-2-yl)ethoxy benzoic acid was prepared as follows:

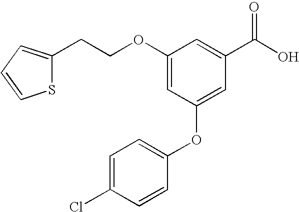

1M NaOH (1.0 ml, 1.0 mM) was added to a solution of methyl 3-(4-chlorophenoxy)-5-(2-thiophen-2-yl)ethoxy benzoate (119 mg, 0.31 mM) in THF (4 ml)/methanol (0.25 ml).

After 17 hr the reaction mixture was neutralised with 1 M citric acid, then concentrated in vacuo. The pH was adjusted to 3-4 with 1M citric acid, extracted with EtOAc (30 ml), washed with brine dried (MgSO₄) and concentrated in vacuo to give 3-(4-chlorophenoxy)-5-(2-thiophen-2-yl)ethoxy benzoic acid as a pale yellow solid (67.5 mg, 58%); ¹H NMR δ (CDCl₃): 3.30 (2H, t), 4.20 (2H, t), 6.79 (1H, m), 6.88 (1H, m), 6.95 (3H, m), 7.16 (1H, d), 7.26-7.40 (4H, br m).

The requisite methyl 3-(4-chlorophenoxy)-5-(2-thiophen-2-yl)ethoxy benzoate was prepared in a manner similar to that given in Tet. Lett. 39 (1998) 2933-2936:

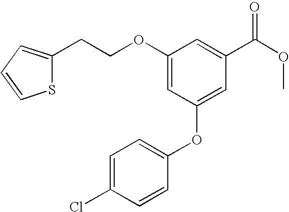

A stirred slurry of methyl 3-hydroxy-5-(2-thiophen-2-yl)ethoxy benzoate (840 mg, 3.0 mM), 4-chlorophenylboronic acid (1.42 g, 9.0 mM), and triethylamine (1.26 ml, 9.0 mM) in toluene (50 ml) was treated with the copper (II) acetate (822 mg, 4.5 mM), and heated to 60° C. for 2 hours under an inert atmosphere, before being left to cool down to room temperature overnight. A further 0.71 g of 4-chlorophenylboronic acid, 0.411 g of copper (II)acetate and 0.63 ml of triethylamine were added and the mixture heated to 110° C. for hours under an inert atmosphere before being cooled to room temperature. The solvent was removed in vacuo and the resulting dark turquoise solid was purified on silica gel (10% EtOAc in isohexane) to give an off white oily solid (119 mg, 10%); ¹H NMR δ (CDCl₃): 3.31 (2H, t), 3.88 (3H, s), 4.22 (2H, t), 6.76 (1H m), 6.91 (1H, m), 6.95 (3H, m), 7.16 (1H, d), 7.23 (1H, m), 7.30 (1H, m), 7.33 (2H, m).

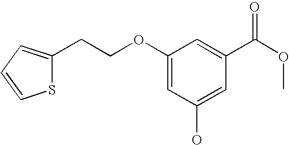

The requisite methyl 3-hydroxy-5-(2-thiophen-2-yl)ethoxy benzoate was prepared using Mitsonobu conditions analagous to the method given in generic Alkylation Method B, to yield the methyl ester as a waxy solid, ¹H NMR δ (d₆ DMSO): 3.25 (2H, t), 3.8 (3H, s), 4.2 (2H, t), 6.6 (1H m), 6.95 (1H, m), 7.0 (3H, m), 7.35 (1H, m), 9.8 (1H, br s).

### EXAMPLE S

The following table lists examples S₁ to S₈₁ which were made using analogous methods to those described above. In this table:

(1) Route refers to method of preparation of final compound, as follows:

| | |
|---|---|
| Route 1 | see Example A; |
| Route 2 | see Example B; |
| Route 3 | see Example C; |
| Route 4 | see Example D; |
| Route 6 | see Example F; |
| Route 7 | see Example G; |
| Route 10 | see Example J; |
| Route 11 | see Example K; |
| Route 12 | see Example L; |
| Route 13 | see Example M; |
| Route 14 | see Example N; |
| Route 15 | see Example O; |
| Route 16 | see Example P; |
| Route 17 | see Example Q; and |
| Route 18 | see Example R. |

(2) Coupling Method (CM) refers to the method used to effect the amide coupling between the alkyl 6-amino nicotinate and the appropriate acid:

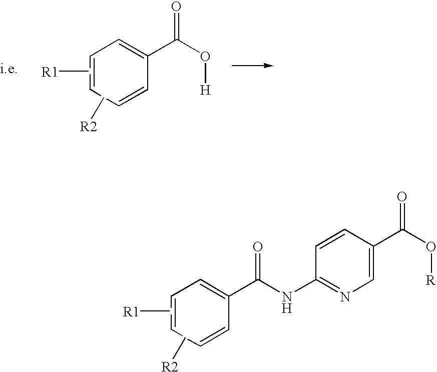

i.e.

(a) Coupling Method A (CM A) refers to Oxalyl chloride coupling, as exemplified in Example A;

(b) Coupling Method B (CM B) refers to EDAC ( ) or similar peptide coupling agent, with or without the addition of a base (eg. di-isopropyl ethylamine or dimethylamino pyridine) or other additives.

For example:

3-isopropyloxy-5-(2-thienyl)methyloxy benzoic acid (740 mg, 2.53 mmol) was dissolved in dry DMF (9 ml), and treated sequentially with dimethyl amino pyridine (900 mg, 7.4 mmol, 3 eq), methyl 6-amino nicotinate (580 mg, 3.8 mmol, 1.5 eq) and EDAC (600 mg, 3.2 mmol, 1.25 eq), and the resulting solution stirred at ambient temperature overnight. The reaction solution was diluted with ethyl actate (100 ml) and the solution washed twice with water, once with citric acid solution (1M) and once with brine, dried (MgSO₄), and evaporated to give methyl 6-[{3-isopropyloxy-5-(2-thienylmethyloxy)benzoyl}amino]-3-pyridinecarboxylate as a pale cream solid (540 mg), MS [MH]⁺ 427, 72% by LC/MS.

(3) Alkylation Method (AM) refers to the generic alkylation method used to synthesise the appropriate acid starting material:

(a) Alkylation Method A (AM A)—synthesis of symmetrical diethers (R1=R2)

For example synthesis of Compound (a)

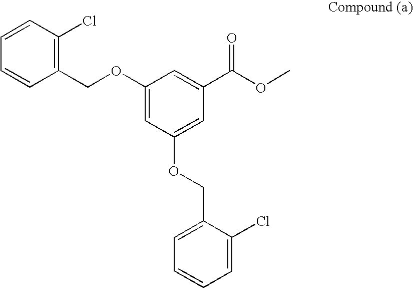

Methyl 3,5-dihydroxybenzoate (74.1 g, 0.44M) was dissolved in dimethylformamide (400 ml), potassium carbonate (152 g, 1.10M) added, stirred for 15 mins then 2-chlorobenzylchloride (117 ml, 0.92M) added and heated at 100° C. under an argon atmosphere. After 3 hrs the reaction mixture was cooled to ambient temperature, concentrated in vacuo, diluted with water (800 ml), extracted with ethyl acetate (2×600 ml). The organic extracts were washed with brine (300 ml), dried (MgSO₄), filtered, concentrated in vacuo to yield a brown oil which was triturated with diethyl ether/isohexane to give compound (a) as an off-white solid (195 g, 100%); ¹H nmr (d6-DMSO, δ values): 3.81 (3H, s); 5.18 (4H, s); 6.98 (1H, m); 7.16 (1H, d); 7.36 (4H, m); 7.50 (2H, m); 7.58 (2H, m).

(b) Alkylation Method B (AM B)—synthesis of unsymmetrical diethers (R1≠R2)

For example, synthesis of compound (b)

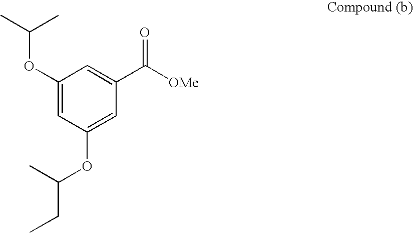

Methyl 3,5-dihydroxybenzoate (16.8 g, 0.1 mol) was dissolved in dimethylformamide (180 ml), powdered potassium carbonate (27.6 g, 0.2 mol) added, followed by 2-iodopropane (10 ml, 0.1 mol), and the resulting suspension stirred overnight at ambient temperature under an argon atmosphere. The reaction mixture was diluted with water (11) and extracted with diethyl ether (2×200 ml). The organic extracts were washed sequentially with water and brine, dried (MgSO₄), filtered and concentrated in vacuo to yield a pale golden oil which was triturated with toluene and filtered to remove unreacted starting material. The filtrate was concentrated in vacuo and the residue chromatographed (2×90 g Biotage cartridges, eluting with isohexane containing ethyl acetate (10% v/v increasing to 15% v/v) to give methyl 3-hydroxy 5-isopropyloxy benzoate as a colourless solid (5.3 g, 25%); ¹H nmr (d6-DMSO, δ values): 1.2 (6H, d); 3.8 (3H, s); 4.6 (1H, hept); 6.55 (1H, m); 6.85 (1H, m); 6.95 (1H, m); 9.8 (1H, s). Methyl 3-hydroxy 5-isopropyloxy benzoate (1.5 g, 7.2 mmol) was dissolved in dimethylformamide (10 ml), potassium carbonate (2.5 g, 18 mmol) added, followed by 2-bromobutane (1.2 ml, 11 mmol), and the resulting suspension stirred for 7 hours at 80 deg C. under an argon atmosphere. The reaction mixture was cooled to ambient temperature, diluted with hexane/ethyl acatate (1:1 v/v) and washed sequentially with water and brine, dried (MgSO₄), filtered and concentrated in vacuo to yield a colourless oil which was chromatographed (flash column on silica (20 g), eluting with isohexane containing ethyl acetate (5% v/v) to give methyl 3-(2-butyloxy) 5-isopropyloxy benzoate as a colourless oil (1.06 g); ¹H nmr (d6-DMSO, δ values): 0.9 (3H, t); 1.2 (3H, d+6H, d); 1.6 (2 h, m); 3.85 (3H, s); 4.4 (1H, hept); 4.55 (1H, hept); 6.7 (1H, m); 7.0 (2H, m); m/z 267 (M+H)+.

(c) Alkylation Method C (AM C)—synthesis of unsymmetrical diethers (R1≠R2)

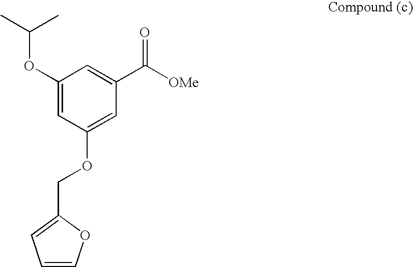

Methyl 3-hydroxy 5-isopropyloxy benzoate (0.5 g, 2.4 mmol) was dissolved in dichloromethane (10 ml) and cooled to 0 deg C. whilst stirring under an argon atmosphere; the solution was treated sequentially with triphenyl phosphine (Polymer supported, 1.19 g, 3.6 mmol), furfuryl alcohol (0.23 ml, 2.7 mmol) and di-t-butyl azodicarboxylate (DtAD, 0.082 g, 3.5 mmol) added dropwise in dichloromethane (4 ml), and the resulting solution stirred for 1.5 hours. The reaction was monitored by hplc and further reagents were added until the starting phenol was consumed—total reagents added were triphenyl phosphine (Polymer supported, 2.38 g, 3 eq), furfuryl alcohol (0.53 ml, 2.5 eq) and DtAD (1.64 g, 3 eq). The reaction mixture was concentrated in vacuo and purified by chromatography (flash column on silica, eluting with isohexane containing ethyl acetate (5% v/v) to give methyl 3-(2-furyl methoxy) 5-isopropyloxy benzoate as a colourless oil, (0.225 g); ¹H nmr (d6-DMSO, δ values): 1.25 (6H, d); 3.85 (3H, s); 4.65 (1H, hept); 5.1 (2H, s); 6.45 (1H, m); 6.6 (1H, m); 6.85 (1H, m); 7.05 (1H, m); 7.15 (1H, m) 7.75 (1H, m).

(d) Alkylation Method D (AM D)—synthesis of unsymmetrical diethers (R1≠R2)

For example, synthesis of Compound (d)

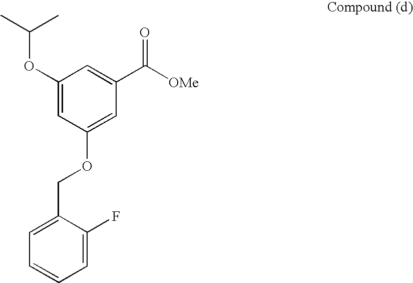

Di-i-propyl azodicarboxylate (DIAD, 0.74 ml, 3.7 mM) was added to methyl (5-isopropoxy-3-hydroxymethyl)-benzoate (0.56 g, 2.5 mM), triphenylphosphine (0.98 g, 3.7 mM) and 2-fluorophenol (0.24 ml, 2.7 mM) in DCM (40 ml) under argon at ambient temperature. After 10 mins concentrated, purified on silica gel (10-15% EtOAc/iso-hexane) gave the title compound as a pale yellow oil, which solidified under high-vacuum (0.71 g, 90%); ¹H NMR δ (d₆-DMSO): 1.26 (d, 6H), 3.82 (s, 3H), 4.64 (m, 1H), 5.21 (s, 2H), 6.92 (m, 1H), 7.09 (m, 1H), 7.16-7.26 (m, 3H), 7.35 (s, 1H), 7.58 (s, 1H).

The above generic methods are for illustration only; it will be appreciated that alternative conditions that may optionally be used include: use of alternative solvents (such as acetone or tetrahydrofuran), alternative stoichiometries of reagents, alternative reaction temperatures and alternative methods of purification.

The esters resulting from the above alkylation methods were hydrolysed using aqueous sodium hydroxide and a water-miscible solvent (eg methanol or THF) in the appropriate quantities, in the manner outlined in Examples C and E.

(4) the letters in parenthesis i.e. ‘(a)’ refer to notes at the bottom of the table

| No | Route | Structure | MS | NMR |
|---|---|---|---|---|
| 1 | 2 (a) | 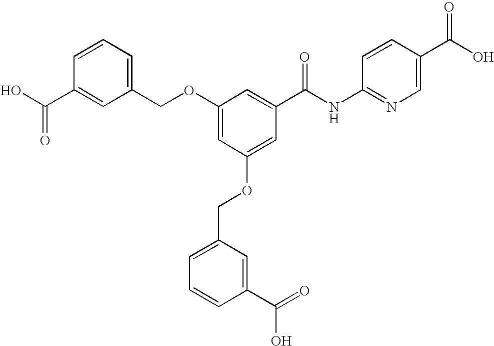 | | δ_{H} (300MHz, DMSO-d₆) 10.96 (1H, s), 8.84 (1H, s), 8.27-8.15 (2H, m), 8.03 (2H, s), 7.88 (2H, d), 7.63 (2H, d), 7.47 (2H, t), 7.35 (2H, s), 6.92 (1H, s), and 5.25 (4H, s). |
| 2 | 3 (a) | 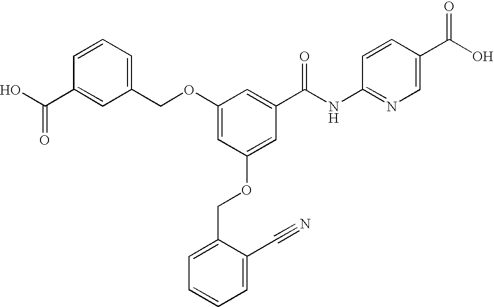 | 524 | — |
| 3 | 3 (b) | 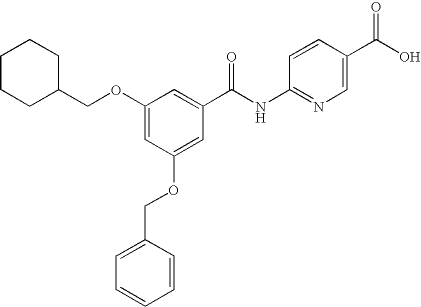 | 461 459 | — |
| 4 | 3 (b) | 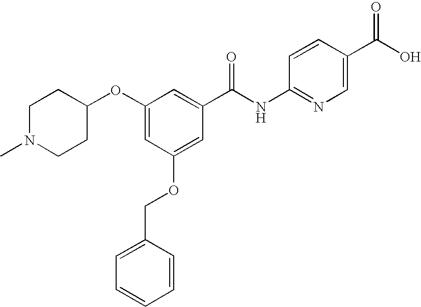 | 462 460 | -s |
| 5 | 3 (b) | 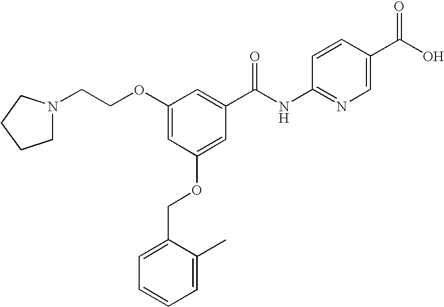 | 476 474 | — |
| 6 | 1 CM A AM C (c) | 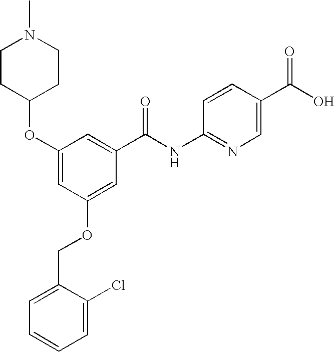 | 496/8 1 ×Cl | ¹H NMR δ (d₆-DMSO): 1.6-1.8 (m, 2H), 1.9-2.0 (m, 2H), 2.7-2.8 (m, 2H), 3.0-3.6 (2H signal obscured by HOD signal), 4.5-4.6 (m, 1H), 5.2 (s, 2H), 6.8 (s, 1H), 7.23 (s, 1H), 7.26 (s, 1H), 7.4 (m, 2H), 7.55 (m, 1H), 7.65 (m, 1H), 8.3 (s, 2H), 8.9 (s, 1H), 11.1 (s, 1H). |
| 7 | 6 (d) | 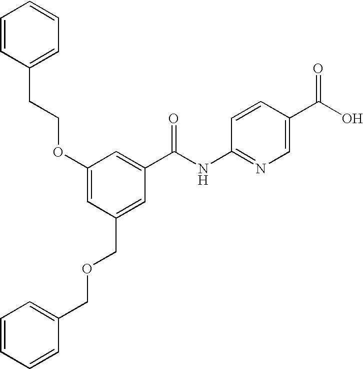 | 483 481 | ¹H NMR δ (d₆-DMSO): 4.58 (s, 4H), 4.62 (s, 4H), 7.25-7.45 (m, 10H), 7.6 (s, 1H), 7.95 (s, 2H), 8.32 (s, 2H), 8.88 (s, 1H), 11.2 (s, IH), 12.88-13.4 (bs, 1H). |
| 8 | 1 (e) | 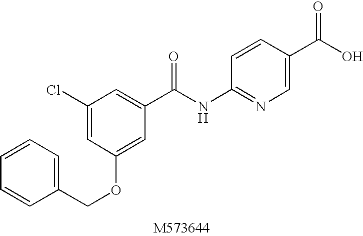 | 383 385 | ¹H NMR δ (d₆-DMSO): 5.22 (s, 2H), 7.30-7.49 (m, 6H), 7.62-7.70 (m, 2H), 8.25-8.35 (m, 2H), 8.65-8.90 (s, 1H), 11.25 (s, 1H), 13.16 (bs, 1H). |
| 9 | 7 | 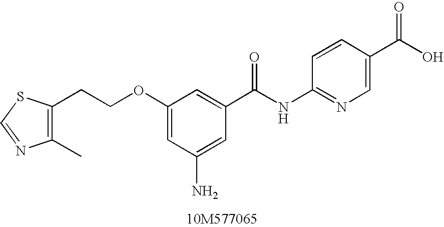 | | ¹H NMR δ (d₆-DMSO): 2.34 (s, 3H), 3.18 (dd, 2H), 4.13 (dd, 2H), 6.31 (m, 1H), 6.80 (m, 2H), 8.25 (s, 2H), 8.82 (s, 1H), 8.85 (s, 1H), 10.80 (bs, 1H). |
| 10 | 8 | 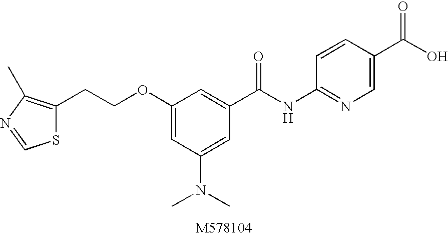 | | ¹H NMR δ (d₆-DMSO): 2.36 (s, 3H), 2.95 (m, 2H), 4.19 (dd, 2H), 6.39 (s, 1H), 6.92 (m, 2H), 6.99 (s, 1H), 8.27 (s, 2H), 8.83 (s, 1H), 8.88 (s, 1H), 11.02 (bs, 1H). |
| 11 | 9 | 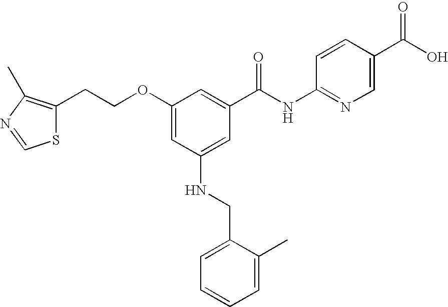 | | ¹H NMR δ (d₆-DMSO): 2.33 (m, 6H), 3.19 (dd, 2H), 4.13 (dd, 2H), 4.26 (s, 2H), 6.33 (s, 1H), 6.83 (s, 1H), 6.90 (s, 1H), 7.09-7.19 (m, 3H), 7.26 (s, 1H), 8.28 (s, 2H), 8.83 (s, 1H), 8.88 (s, 1H), 10.87 (s, 1H), 13.09 (bs, 1H). |
| 12 | 1 CM A AM C | 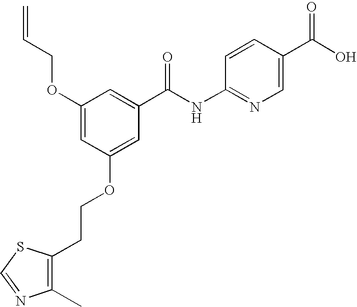 | | ¹H NMR δ (d₆-DMSO): 2.37 (s, 3H), 3.24 (dd, 2H), 4.20 (dd, 2H), 4.66 (d, 2H), 5.27 (d, 1H), 5.40 (d, 1H), 6.06 (m, 1H), 6.73 (s, 1H), 7.22 (s, 2H), 8.31 (s, 2H), 8.86 (m, 2H), 11.12 (s, 1H), 13.15 (bs, 1H). |
| 13 | 1 CM A AM B | 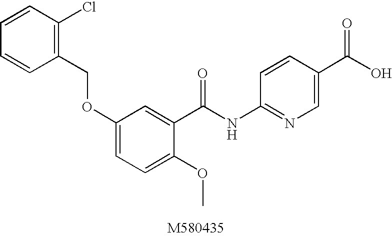 | 427 | ¹H NMR δ (d₆-DMSO): 3.82 (s, 3H), 3.91 (s, 3H), 5.18 (s, 2H), 7.20-7.28 (m, 2H), 7.32 -7.40 (m, 2H), 7.45-7.52 (m, 2H), 7.57-7.61 (m, 1H), 8.35 (s, 2H), 8.84 (s, 1H), 10.56 (s, 1H). |
| 14 | 1 CM A AM B | 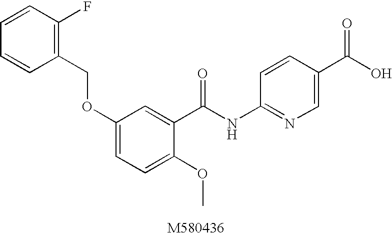 | 397 395 | ¹H NMR δ (d₆-DMSO): 3.94 (s, 3H), 5.18 (s, 2H), 7.18-7.28 (m, 4H), 7.38-7.42 (m, 1H), 7.50-7.58 (m, 2H), 8.30 (s, 2H), 8.81 (s, 1H), 10.73 (s, 1H). |
| 15 | 1 CM A AM B | 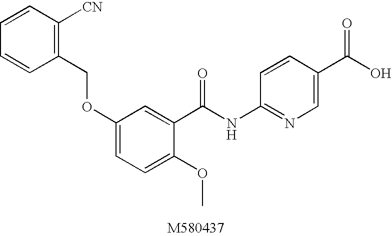 | 404 402 | ¹H NMR δ (d₆-DMSO): 3.95 (s, 3H), 7.21-7.33 (m, 2H), 7.53-7.59 (m, 2H), 7.65-7.72 (m, 2H), 7.89 (d, 1H), 8.27-8.36 (m, 2H), 8.83 (s, 1H), 10.78 (s, 1H). |
| 16 | 1 CM A AM B | 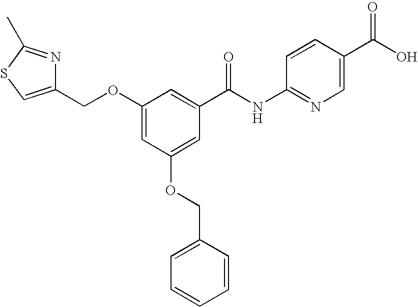 | | ¹H NMR δ (d₆-DMSO): 2.65 (s, 3H), 5.17 (s, 4H), 6.87 (m, 1H), 7.32 (m, 3H), 7.37 (m, 2H), 7.43 (m, 2H), 7.52 (s, 1H), 8.29 (m, 2H), 8.87 (s, 1H), 11.15 (s, 1H). |
| 17 | 1 CM A AM A | 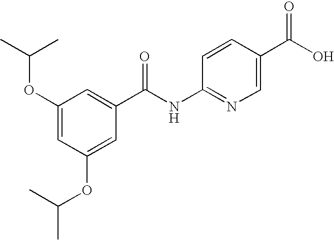 | 359 | ¹H NMR δ (d₆-DMSO): 1.13 (d, 12H), 4.62-4.72 (m, 2H), 6.61 (s, 1H), 7.14 (s, 2H), 8.27 (s, 2H), 8.84 (s, 1H), 11.08 (s, 1H). |
| 18 | 1 CM A AM A | 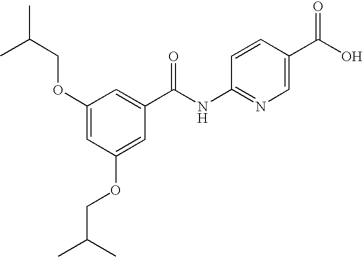 | 387 385 | ¹H NMR δ (d₆-DMSO): 0.98 (d, 12H), 1.96-2.14 (m, 1H), 3.81 (d, 4H), 6.63 (s, 1H), 7.19 (s, 2H), 8.27 (s, 2H), 8.82 (s, 1H), 11.18 (s, 1H), 13.25 (br s, 1H). |
| 19 | 1 CM A AM B | 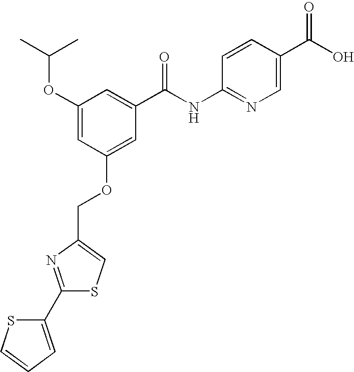 | | ¹H NMR δ (d₆-DMSO): 1.28 (d, 6H), 4.73 (m, 1H), 5.27 (s, 2H), 6.82 (s, 1H), 7.15 (t, 1H), 7.21 (s, 1H), 7.33 (s, 1H), 7.67 (m, 1H), 7.73 (m, 2H), 8.32 (s, 2H), 8.88 (s, 1H), 11.18 (s, 1H). |
| 20 | 1 CM A AM B | 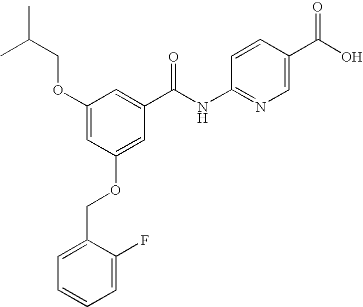 | 439 437 | ¹H NMR δ (d₆-DMSO): 0.98 (d, 12H), 1.97-2.14 (m, 1H), 3.80 (d, 4H), 5.20 (s, 2H), 6.80 (s, 1H), 7.19-7.25 (m, 3H), 7.31 (s, 1H), 7.39-7.43 (m, 1H), 7.57 (t, 1H), 8.28 (s, 2H), 8.84 (s, 1H), 11.12 (s, 1H). |
| 21 | 1 CM A AM B | 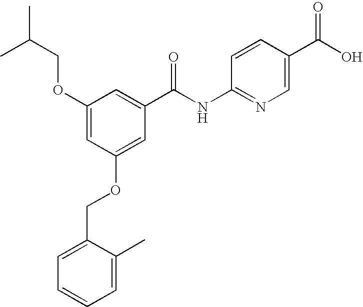 | 433 | ¹H NMR δ (d₆-DMSO): 0.99 (d, 6H), 1.97-2.14 (m, 1H), 2.32 (s, 3H), 3.80 (d, 2H), 5.16 (s, 2H), 6.80 (s, 1H), 7.19-7.23 (m, 4H), 7.31 (s, 1H), 7.39-7.42 (m, 1H), 8.30 (s, 2H), 8.84 (s, 1H), 11.10 (s, 1H). |
| 22 | 1 CM A AM B | 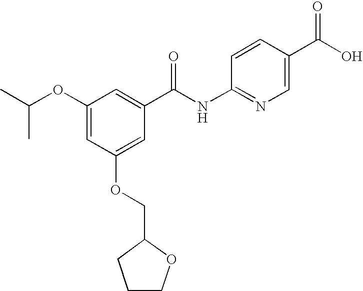 | | ¹H NMR δ (d₆-DMSO): 1.33 (d, 6H), 1.67-1.78 (m, 1H), 1.86-2.12 (m, 3H), 3.73 (m, 2H), 3.84 (m, 2H), 4.01-4.11 (m, 2H), 4.22 (m, 1H), 4.78 (m, 1H), 6.73 (s, 1H), 7.23 (m, 2H), 8.38 (s, 2H), 8.94 (s, 1H), 11.20 (s, 1H). |
| 23 | 1 CM A AM B | 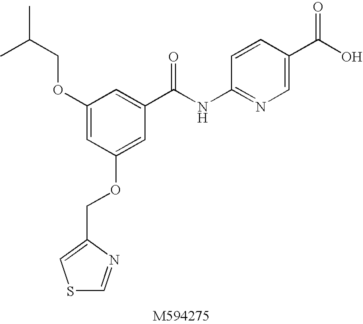 | 428 426 | ¹H NMR δ (d₆-DMSO): 0.99 (d, 6H), 1.97-2.13 (m, 1H), 3.80 (d, 2H), 5.28 (s, 2H), 6.80 (s, 1H), 7.21 (s, 1H), 7.31 (s, 1H), 7.78 (s, 1H), 8.30 (s, 2H), 8.84 (s, 1H), 9.10 (s, 1H), 11.10 (s, 1H). |
| 24 | 1 CM A AM B | 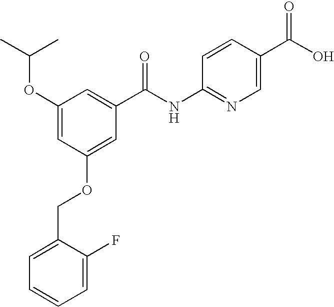 | | ¹H NMR δ (d₆-DMSO): 1.26 (d, 6H), 4.71 (m, 1H), 5.20 (s, 2H), 6.75 (m, 1H), 7.18-7.32 (m, 4H), 7.42 (m, 1H), 7.53 (m, 1H), 8.29 (m, 2H), 8.87 (s, 1H), 11.10 (s, 1H). |
| 25 | 1 CM A AM B | 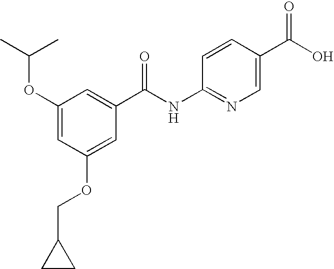 | 371 369 | ¹H NMR δ (d₆-DMSO): 0.01 (d, 2H), 0.23 (d, 2H), 0.90-0.99 (m, 1H), 0.98 (d, 6H), 3.79 (d, 2H), 4.48-5.12 (m, 1H), 6.36 (s, 1H), 6.83 (s, 2H), 8.00 (s, 2H), 8.58 (s, 1H), 10.77 (s, 1H). |
| 26 | 1 CM A AM B | 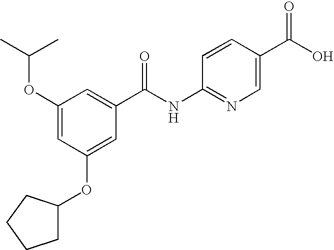 | 385 383 | ¹H NMR δ (d₆-DMSO): 1.12 (d, 6H), 1.52-1.61 (m, 2H), 1.60-1.78 (m, 4H), 1.82-1.97 (m, 2H), 4.65-4.75 (m, 1H), 4.88 (br t, 1H), 6.60 (s, 1H), 7.14 (d, 2H), 8.24 (s, 2H), 8.83 (s, 1H) 11.07 (s, 1H). |
| 27 | 1 CM A AM B | 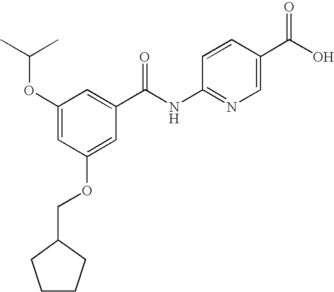 | 399 397 | ¹H NMR δ (d₆-DMSO): 1.12 (d, 6H), 1.12-1.38 (m, 2H), 1.43-1.61 (m, 4H), 1.68-1.80 (m, 2H), 2.12-2.36 (m, 1H), 3.86 (d, 2H), 4.65-4.75 (m, 1H), 6.61 (s, 1H), 7.18 (s, 2H), 8.24 (s, 2H), 8.83 (s, 1H), 11.07 (br s, 1H). |
| 28 | 1 CM A AM B | 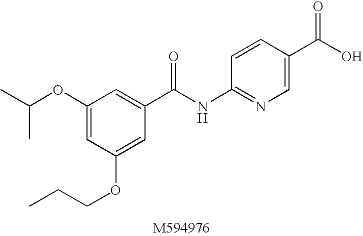 | 359.4 | ¹H NMR δ (d₆-DMSO): ): 1.98 (t, 3H), 1.25 (d, 6H), 1.65-1.82 (m, 2H), 4.00 (t, 2H), 4.66-4.79 (m, 1H), 6.65 (m, 1H), 7.18 (m, 2H), 8.32 (m, 2H), 8.89 (m, 1H), 11.12 (s, 1H), 13.12 (bs 1H) |
| 29 | 1 CM A AM B | 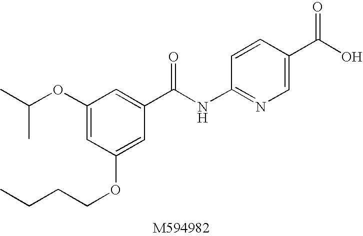 | 372 | ¹H NMR δ (d₆-DMSO): 0.95 (t, 3H), 1.27 (d, 6H), 1.35-1.54 (m, 2H), 1.61-1.80 (m, 2H), 4.03 (t, 2H), 4.654.79 (m, 1H), 6.65 (m, 1H), 7.18 (m, 2H), 8.32 (m, 2H), 8.89 (m, 1H) 11.15 (s, 1H), 13.2 (bs, 1H) |
| 30 | 1 CM A AM B | 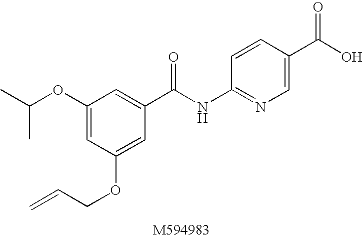 | 357.4 | ¹H NMR δ (d₆-DMSO): 1.26 (d, 6H), 4.65 (d, 2H), 4.67-4.80 (m, 1H), 5.26 (d, 1H), 5.42 (d, 1H), 5.95-6.15 (m, 1H), 6.70 (s, 1H), 7.20 (s, 2H), 8.32 (s, 2H), 8.89 (s, 1H), 11.15 (s, 1H), 13.20 (bs, 1H) |
| 31 | 1 CM A AM B (h) | 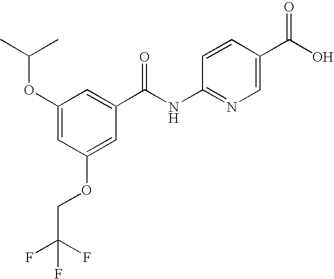 | | ¹H NMR (d₆-DMSO): 1.27 (d, 6H), 4.75 (m, 1H), 4.82 (q, 2H), 6.81 (2, 1H), 7.26 (s, 1H), 7.30 (s, 1H), 8.30 (s, 2H), 8.88 (s, 1H), 11.09 (s, 1H) |
| 32 | 1 CM A AM C | 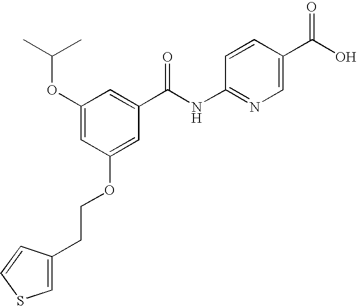 | | ¹H NMR δ (d₆-DMSO): 1.26 (d, 6H), 3.05 (dd, 2H), 4.25 (dd, 2H), 4.69 (m, 1H), 6.66 (m, 1H), 7.11 (d, 2H), 7.16 (s, 1H), 7.20 (s, 1H), 7.30 (m, 1H), 7.45 (dd, 1H), 8.27 (s, 2H), 8.85 (s, 1H), 11.09 (s, 1H). |
| 33 | 1 CM A AM B | 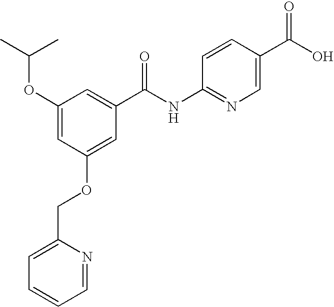 | | ¹H NMR δ (d₆-DMSO): 1.26 (d, 6H), 4.72 (m, 1H), 5.24 (s, 2H), 6.76 (m, 1H), 7.18 (s, 2H), 7.29 (s, 1H), 7.34 (m, 1H), 7.53 (d, 2H), 7.82 (td, 1H), 8.28 (m, 2H), 8.57 (m, 1H), 8.87 (s, 1H), 11.11 (s, 1H). |
| 34 | 10 | 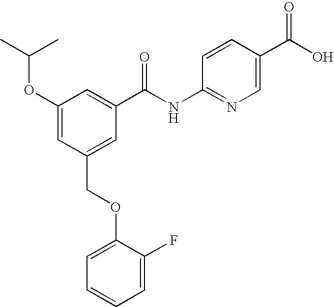 | | ¹H NMR δ (d₆-DMSO): 1.28 (d, 6H), 4.74 (m, 1H), 5.20 (s, 2H), 6.87-6.97 (m, 1H), 7.10 (m, 1H), 7.16-7.26 (m, 3H), 7.54 (s, 1H), 7.66 (s, 1H), 8.28 (s, 2H), 8.84 (s, 1H), 11.78 (bs, 1H). |
| 35 | 1 CM A AM C | 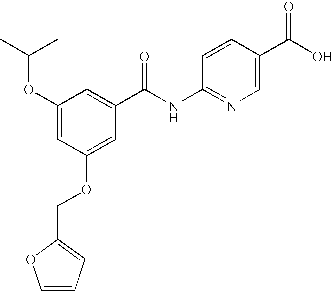 | | ¹H NMR δ (d₆-DMSO): 1.26 (d, 6H), 4.71 (m, 1H), 5.10 (s, 2H), 6.45 (m, 1H), 6.56 (m, 1H), 6.74 (m, 1H), 7.18 (s, 1H), 7.26 (s, 1H), 7.66 (m, 1H), 8.29 (m, 2H), 8.87 (s, 1H). |
| 36 | 1 CM A AM B | 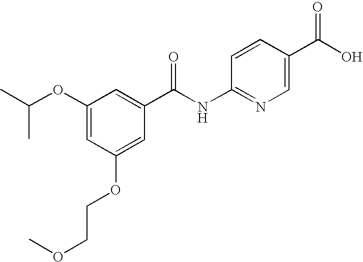 | 373 375 | ¹H NMR δ (d₆-DMSO): 1.25 (d, 6H), 3.35 (s, 3H), 3.7 (m, 2H), 4.15 (m, 2H), 4.74 (m, 1H), 6.7 (t, 1H), 7.2 (s, 2H), 8.3 (s, 2H), 8.9 (s, 1H), 11.15 (s, 1H), 13.2 (br s, 1H). |
| 37 | 1 CM A AM B | 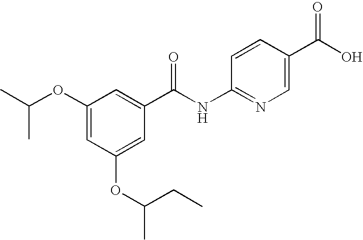 | 371 373 | ¹H NMR δ (d₆-DMSO): 0.95 (t, 3H), 1.25 (d, 6H +t, 3H), 1.65 (m, 2H), 4.5 (hept, 1H), 4.75 (hept, 1H), 6.65 (t, 1H), 7.2 (s, 2H), 8.3 (s, 2H), 8.9 (s, 1H), 11.15 (s, 1H), 13.2 (br s, 1H). |
| 38 | 1 CM A AM B | 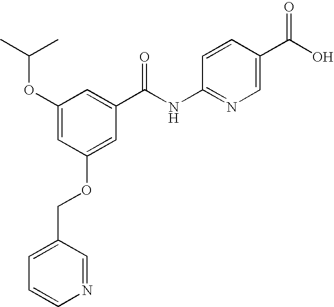 | | ¹H NMR δ (d₆-DMSO): 1.26 (d, 6H), 4.71 (m, 1H), 5.21 (s, 2H), 6.76 (m, 1H), 7.21 (s, 1H), 7.30 (s, 1H), 7.42 (m, 1H), 7.87 (m, 1H), 8.28 (m, 2H), 8.53 (m, 1H), 8.67 (s, 1H), 8.87 (s, 1H), 11.10 (s, 1H). |
| 39 | 1 CM A AM B | 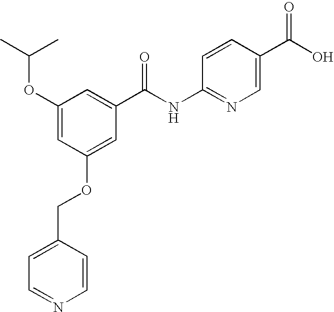 | | ¹H NMR δ (d₆-DMSO): 1.24 (d, 6H), 4.71 (m, 1H), 5.24 (s, 2H), 6.76 (m, 1H), 7.43 (m, 2H), 7.67 (m, 2H), 8.27 (m, 2H), 8.56 (m, 2H), 8.87 (s, 1H), 11.06 (bs, 1H). |
| 40 | 1 CM A AM B | 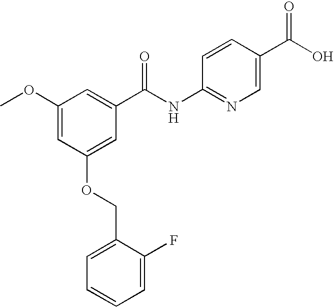 | 395 397 | ¹H NMR δ (d₆-DMSO): 3.85 (s, 3H), 5.25 (s, 2H), 6.85 (t, 1H), 7.2-7.3 (m, 3H), 7.35 (s, 1H), 7.45 (m, 1H), 7.6 (t of d, 1H), 8.3 (s, 2H), 8.9 (s, 1H), 11.15 (s, 1H), 13.2 (br s, 1H). |
| 41 | 12 | 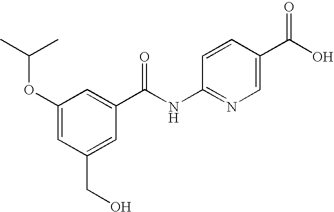 | | ¹H NMR δ (d₆-DMSO): 1.28 (d, 6H), 4.50 (s, 2H), 4.72 (m, 1H), 7.06 (s, 1H), 7.42 (s, 1H), 7.53 (s, 1H), 8.29 (s, 2H), 8.87 (s, 1H), 11.09 (bs, 1H). |
| 42 | 1 CM A AM B | 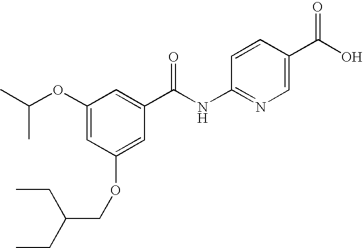 | 401 399 | ¹H NMR δ (d₆-DMSO 0.9 (t, 6H), 1.27-1.35 (d, 6H), 1.35-1.54 (m, 4H), 1.57-1.67 (m, 1H), 3.95 (d, 2H), 4.67-4.78 (m, 1H), 6.67 (m, 1H), 7.19 (m, 2H), 8.30 (app s, 2H), 8.90 (app s, 1H), 11.09 (s, 1H), 13.15 (s, 1H) |
| 43 | See Example K | 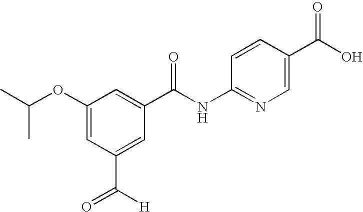 | | ¹H NMR δ (d₆-DMSO): 1.32 (d, 6H), 4.82 (m, 1H), 7.58 (m, 1H), 7.84 (m, 1H), 8.11 (s, 1H), 8.29 (s, 2H), 8.87 (s, 1H), 10.02 (s, 1H), 11.34 (bs, 1H). |
| 44 | 11 | 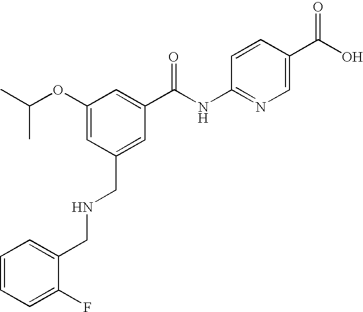 | | ¹H NMR δ (d₆-DMSO): 1.29 (d, 6H), 4.13 (d, 2H), 4.74 (m, 1H), 7.20-7.30 (m, 3H), 7.43 (m, 1H), 7.58 (m, 2H), 7.68 (s, 1H), 8.28 (s, 2H), 8.87 (s, 1H), 11.10 (bs, 1H). |
| 45 | See Example M | 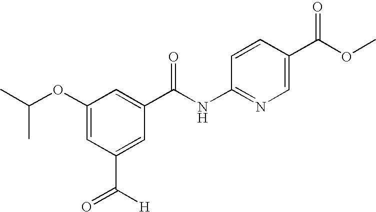 | | ¹H NMR δ (d₆-DMSO): 1.32 (d, 6H), 3.85 (s, 3H), 4.82 (m, 1H), 7.58 (m, 1H), 7.84 (m, 1H), 8.08 (s, 1H), 8.32 (s, 2H), 8.89 (s, 1H), 10.02 (s, 1H), 11.40 (bs, 1H). |
| 46 | 11 | 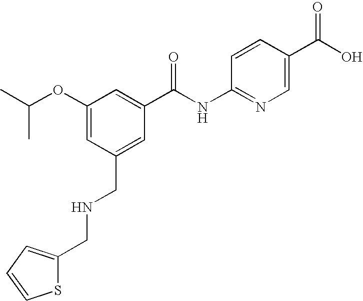 | | ¹H NMR δ (d₆-DMSO): 1.30 (d, 6H), 4.13 (s, 2H), 4.35. (s, 2H), 4.75 (m, 1H), 7.08 (m, 1H), 7.29 (m, 2H), 7.59 (m, 2H), 7.68 (s, 1H), 8.29 (s, 2H), 8.87 (s, 1H), 11.10 (bs, 1H). |
| 47 | 13 | 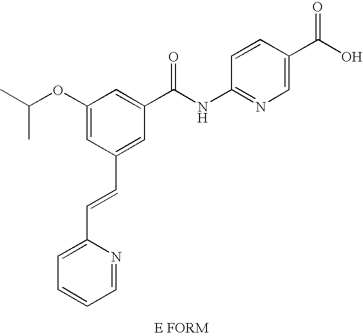 | | ¹H NMR δ (d₆-DMSO): 1.32 (d, 6H), 4.82 (m, 1H), 7.40 (s, 1H), 7.49-7.58 (m, 1H), 7.61 (d, 1H), 7.62 (m, 1H), 7.72 (m, 1H), 7.91 (s, 1H), 8.03 (d, 1H), 8.13 (d, 1H), 8.32 (m, 2H), 8.74 (m, 1H), 8.89 (m, 1H), 11.28 (bs, 1H). |
| 48 | 1 (f) | 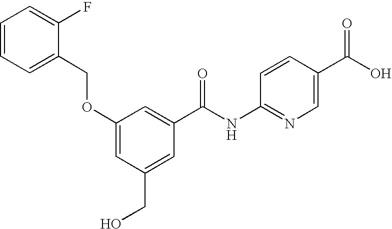 | 395 | ¹H NMR δ (d₆-DMSO): 4.53 (s, 2H), 5.22 (s, 2H), 5.20-5.38 br s 1H), 7.18-7.28 (m, 3H), 7.38-7.42 (m, 1H), 7.52-7.62 (m, 3H), 8.32 (s, 2H), 8.84 (s, 1H), 11.11 (s, 1H). |
| 49 | 1 CM A AM C | 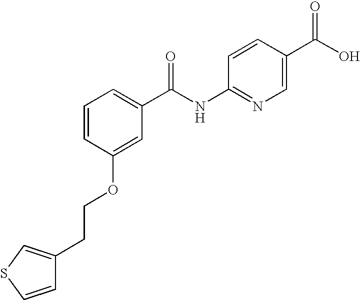 | 369.11 367.14 | ¹H NMR (d₆-DMSO): 3.08 (t, 2H), 4.29 (t, 2H), 7.15 (m, 2H), 7.32 (s, 1H), 7.41 (t, 1H), 7.46 (m, 1H), 7.61 (m, 2H), 8.30 (s, 2H), 8.87 (s, 1H), 11.12 (s, 1H), 13.06 (bs, 1H) |
| 50 | 14 | 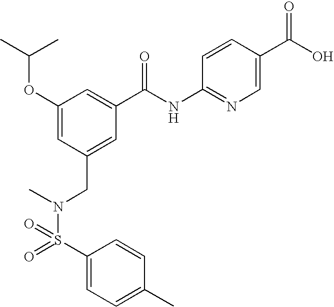 | 498 496 | ¹H NMR δ (d₆-DMSO): 1.23 (d, 6H), 2.40 (s, 3H), 2.58 (s, 3H), 4.13 (s, 2H), 4.62-4.72 (m, 1H), 7.70 (s, 1H), 7.41-7.52 (m, 4H), 7.73 (d, 2H), 8.31 (s, 2H), 8.84 (s, 1H), 11.16 (s, 1H). |
| 51 | 1 CM B AM C | 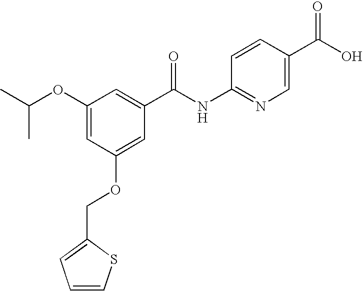 | 411 | ¹H NMR δ (d₆-DMSO): 1.25 (d, 6H), 4.7 (m, 1H), 5.35 (s, 2H), 6.5 (s, 1H), 7.0 (m, 1H), 7.2 (s, 2H), 7.3 (s, 1H), 7.55 (d, 1H), 8.3 (s, 2H), 8.9 (s, 1H), 11.1 (br s, 1H). |
| 52 | 1 CM B AM C | 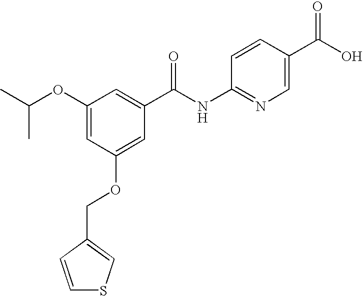 | | ¹H NMR δ (d₆-DMSO): 1.25 (d, 6H), 4.7 (m, 1H), 5.15 (s, 2H), 6.75 (s, 1H), 7.2 (m, 2H), 7.3 (s, 1H), 7.55-7.6 (m, 2H), 8.3 (s, 2H), 8.9 (s, 1H), 11.1 (br s, 1H). |
| 53 | 4 | 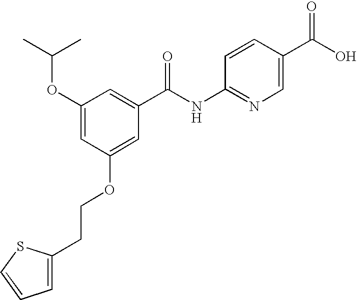 | 427.38 | ¹H NMR (d₆-DMSO): 1.27 (d, 6H), 3.26 (ap t, 2H), 4.26 (t, 2H), 4.71 (m, 1H), 6.67 (s, 1H), 2H), 7.34 (d, 1H), 8.29 11.11 (s, 1H) |
| 54 | 10 | 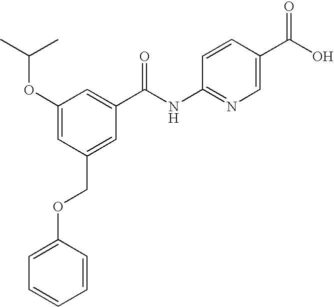 | 407 405 | ¹H NMR δ (d₆-DMSO): 1.15 (d, 6H), 4.69-4.80 (m, 1H), 5.14 (s, 2H), 6.95 (t, 1H), 7.01 (d, 2H), 7.18 (s, 1H), 7.26 (t, 2H), 7.52 (s, 1H), 7.63 (s, 1H), 8.30 (s, 2H), 8.84 (s, 1H), 11.13 (s, 1H). |
| 55 | 10 | 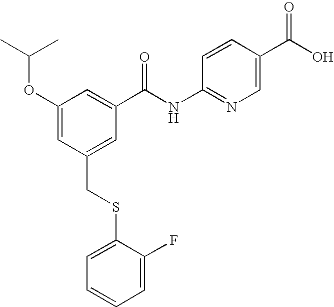 | 441 439 | ¹H NMR δ (d₆-DMSO): 1.15 (d, 6H), 4.22 (s, 2H), 4.61-4.71 (m, 1H), 7.08 (s, 1H), 7.10-7.20 (m, 2H), 7.20-7.28 (m, 1H), 7.41-7.48 (m, 2H), 7.59 (s, 1H), 8.28 (s, 2H), 8.84 (s, 1H), 11.09 (s, 1H). |
| 56 | 10 (f), (g) | 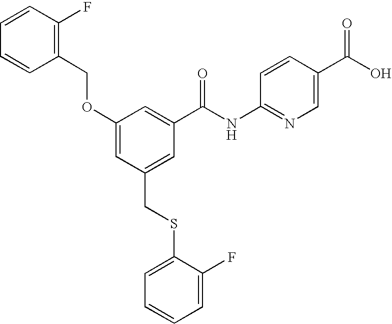 | 507 505 | ¹H NMR δ (d₆-DMSO): 4.22 (s, 2H), 5.20 (s, 2H), 7.10-7.30 (m, 6H), 7.39-7.44 (m, 2H), 7.56 (t, 1H), 7.62 (s, 2H), 8.30 (s, 2H), 8.84 (s, 1H), 11.11 (s, 1H). |
| 57 | 1 CM B AM B | 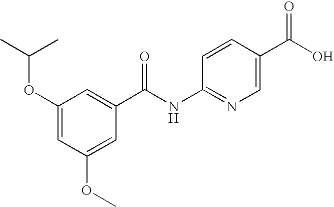 | 331 329 | δ_{H} (300MHz, DMSO-d₆) 1.25 (6H, d), 3.8 (3H, s), 4.7 (1H, hept), 6.65 (1H, m), 7.2 (2H, m), 8.3 (2H, s), 8.9 (1H, s), 11.1 (1H, br s), 13.1 (1H, br s). |
| 58 | 4 | 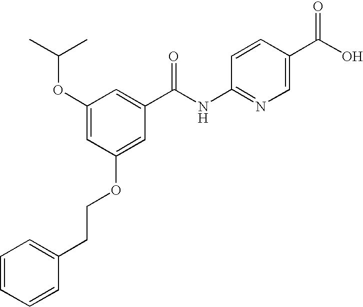 | | ¹H NMR (d₆-DMSO): 1.27 (d, 6H), 3.04 (t, 2H), 4.26 (t, 2H), 4.70 (m, 1H), 6.65 (s, 1H), 7.14-7.38 (m, 7H), 8.29 (s, 2H), 8.87 (s, 1H), 11.09 (s, 1H) |
| 59 | 4 | 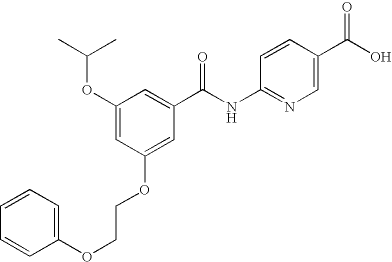 | | δ_{H} ¹H NMR (d₆- DMSO): 1.28 (d, 6H), 4.32 (m, 2H), 4.39 (m, 2H), 4.72 (m, 1H), 6.72 (s, 1H), 6.88-7.02 (m, 3H), 7.19 (s, 1H), 7.22-7.34 (m, 3H), 7.30 (s, 2H), 8.88 (s, 1H), 11.11 (s, 1H) |
| 60 | 15 | 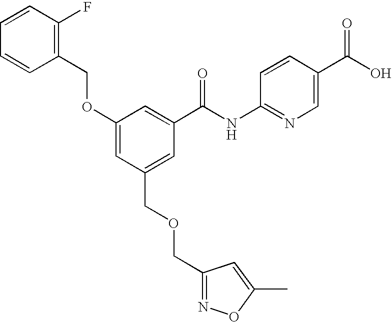 | 492 490 | δ_{H} (300MHz, d₆-dmso) 2.40 (s, 3H); 4.58 (s, 4H), 5.22 (s, 2H); 6.26 (s, 1H); 7.21-7.30 (m, 3H); 7.38-7.45 (m, 1H); 7.55-7.60 (ap d, 1H); 7.60 (s, 1H); 7.64 (s, 1H); 8.32 (s, 2H); 8.86 (s, 1H); 11.16 (br s, 1H) |
| 61 | 4 | 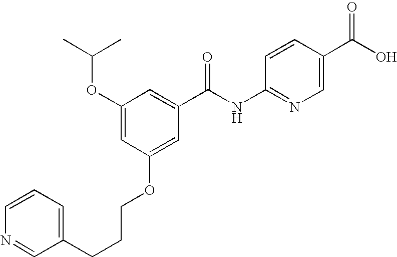 | | δ_{H} ¹H NMR (d₆- DMSO): 1.27 (d, 6H), 2.04 (m, 2H), 2.78 (t, 2H), 4.03 (t, 2H), 4.72 (m, 1H), 6.65 (s, 1H), 7.18 (s, 2H), 7.30 (dd, 1H), 7.66 (d, 1H), 8.29 (s, 2H), 8.39 (d, 1H), 8.46 (s, 1H), 8.88 (s, 1H), 11.08 (s, 1H) |
| 62 | 16 | 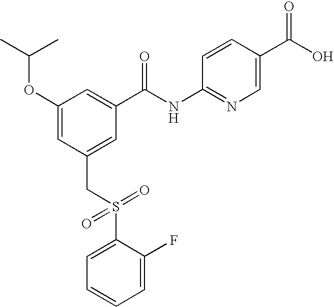 | 473 471 | (300MHz, d₆-dmso) 1.12 (d, 6H); 4.58-4.66 (m, 1H); 4.79 (s, 2H); 6.98 (s, 1H); 7.30-7.41 (m, 2H); 7.43 (s, 1H); 7.48-7.63 (m, 2H); 7.72-7.81 (m, 1H); 8.30 (s, 2H); 8.86 (S, 1H); 11.08 (br s, 1H) |
| 63 | 1 CM C AM A (i) | 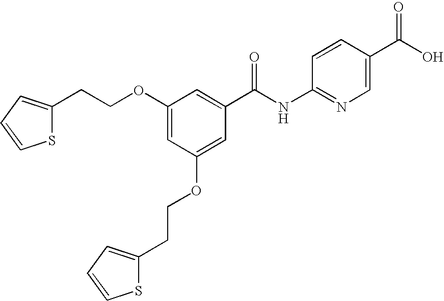 | 493 495 95% | δ_{H} (300MHz, DMSO-d₆) 3.25 (4H, t, obscured by HOD signal), 4.25 (4H, t), 6.75 (1H, m), 6.95 (4H, m), 7.25 (2H, d), 7.35 (2H, d), 8.3 (2H, d), 8.85 (1H, d), 11.1 (1H, br s). |
| 64 | 1 CM C AM C (j) | 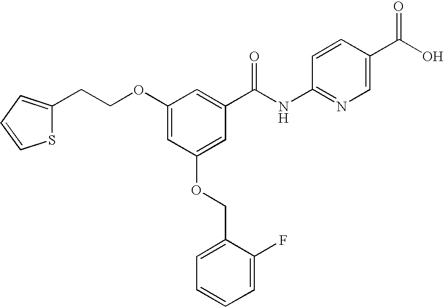 | 491 493 98% | δ_{H} (300MHz, DMSO-d₆) 3.25 (2H, t, obscured by HOD signal), 4.25 (2H, t), 5.2 (2H, s), 6.8 (1H, m), 7.0 (2H, m), 7.25 (3H, m), 7.35 (2H, m), 7.4 (1H, m), 7.6 (1H, m), 8.3 (2H, m), 8.85 (1H, d), 11.1 (1H, br s). |
| 65 | 1 CM C AM C (j) | 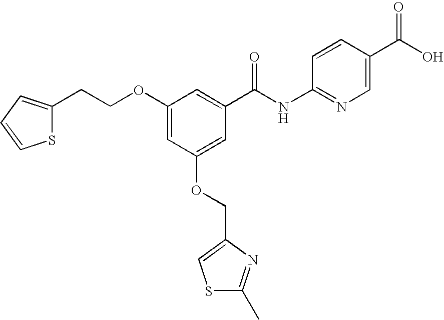 | 494 496 98% | δ_{H} (300MHz, DMSO-d₆) 2.65 (3H, s), 3.25 (2H, t, obscured by HOD signal), 4.25 (2H, t), 5.2 (2H, s), 6.85 (1H, s), 6.95 (2H, m), 7.25 (1H, s), 7.35 (2H, m), 7.55 (1H, s), 8.3 (2H, m), 8.9 (1H, m), 11.1 (1H, br s). |
| 66 | 8 | 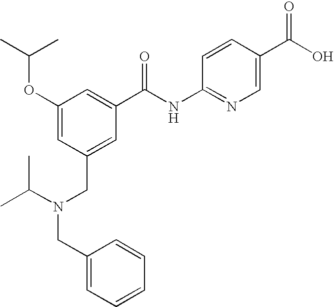 | 462 460 | |
| 67 | 1 CM C AM C (j) | 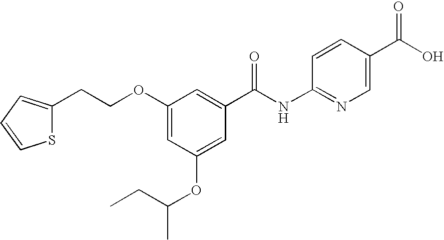 | 439 441 97.7% | δ_{H} (300MHz, DMSO-d₆) 0.9 (3H, t), 1.1 (3H, d), 1.6 (2H, m), 3.25 (2H, t, obscured by HOD signal), 4.25 (2H, t), 4.5 (1H, hex), 6.65 (1H, m), 7.0 (2H, m), 7.2 (2H, d), 7.35 (1H, d), 8.3 (2H, s), 8.9 (1H, s), 11.1 (1H, br s). |
| 68 | 1 CM C AM C (j) | 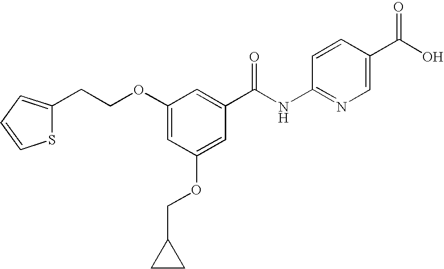 | 437 439 98% | δ_{H} (300MHz, DMSO-d₆) 0.0 (2H, m), 0.25 (2H, m), 0.95 (1H, m), 3.0 (2H, t, obscured by HOD signal), 3.6 (2H, d), 4.0 (2H, t), 6.4 (1H, m), 6.7 (2H, m), 6.9 (2H, d), 7.05 (1H, m), 8.0 (2H, s), 8.6 (1H, s), 10.8 (1H, br s). NB Spectrum shifted by approx 0.3 ppm. |
| 69 | 1 CM C AM C | 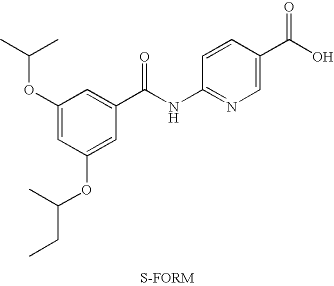 | 373 371 | δ_{H} (300MHz, DMSO-d₆) 0.92 (3H, t), 1.22 (3H, d), 1.28 (6H, d), 1.53-1.69 (2H, br m), 4.49 (1H, m), 4.71 (1H, m), 6.62 (1H, m), 7.15 (2H, m), 8.28 (2H, m), 8.87 (1H, s), 11.08 (1H, br s). |
| 70 | 1 CM C AM C | 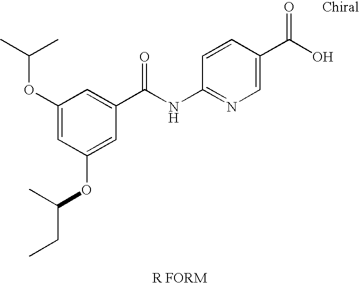 | 373 371 | δ_{H} (300MHz, DMSO-d₆) 0.92 (3H, t), 1.23 (3H, d), 1.28 (6H, d), 1.53-1.71 (2H, br m), 4.48 (1H, m), 4.72 (1H, m), 6.63 (1H, m), 7.15 (2H, m), 8.28 (2H, m), 8.87 (1H, s), 11.08 (1H, br s). |
| 71 | 17 | 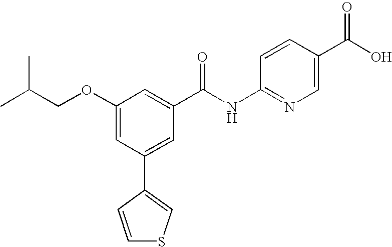 | 397 395 | — |
| 72 | 4 | 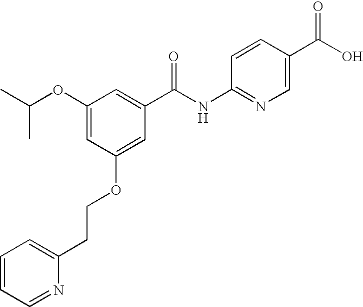 | 422 420 | ¹H NMR (d₆-DMSO): 1.26 (d, 6H), 3.19 (t, 2H), 4.42 (t, 2H), 4.70 (m, 1H), 6.64 (s, 1H), 7.17 (d, 2H), 7.23 (m, 1H), 7.37 (d, 1H), 7.72 (t, 1H), 8.29 (s, 2H), 8.50 (d, 1H), 8.86 (s, 1H), 11.10 (s, 1H) |
| 73 | 14 | 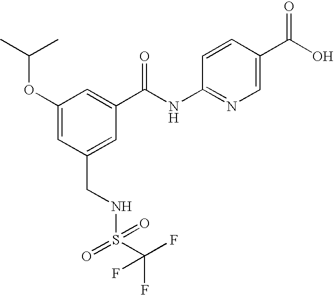 | 462 460 | (400MHz, d₆-dmso) 1.3 (d, 6H); 4.39 (d, 2H); 4.70-4.78 (m, 1H); 7.09 (s, 1H); 7.55 (d, 2H); 8.31 (s, 2H); 8.88 (s, 1H); 9.95 (t, 1H); 11.14 (s, 1H), 13.18 (br s, 1H). |
| 74 | 4 | 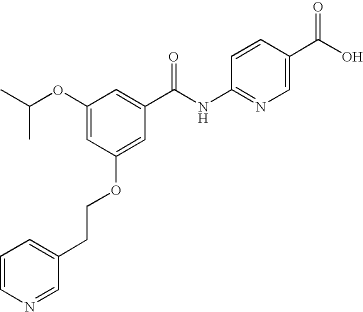 | 422.45 420.42 | δ_{H} ¹H NMR (d₆- DMSO): 1.26 (d, 6H), 3.06 (t, 2H), 4.28 (t, 2H), 4.70 (m, 1H), 6.66 (s, 1H), 7.18 (d, 2H), 7.34 (dd, 1H), 7.76 (d, 1H), 8.29 (s, 2H), 8.43 (d, 1H), 8.55 (s, 1H), 8.86 (s, 1H), 11.08 (s, 1H) |
| 75 | 4 | 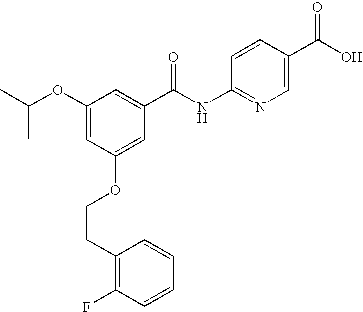 | 439.43 437.42 | δ_{H} ¹H NMR (d₆- DMSO): 1.28 (d, 6H), 3.10 (t, 2H), 4.28 (t, 2H), 4.72 (m, 1H), 6.67 (s, 1H), 7.19 (m, 4H), 7.31 (m, 1H), 7.44 (t, 1H), 8.31 (s, 2H), 8.89 (s, 1H), 11.11 (s, 1H) |
| 76 | 4 | 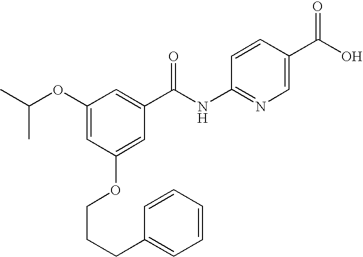 | 435.45 433.44 | δ_{H} ¹H NMR (d₆- DMSO): 1.29 (d, 6H), 2.04 (m, 2H), 2.77 (t, 2H), 4.05 (t, 2H), 4.73 (m, 1H), 6.68 (s, 1H), 7.20 (s, 3H), 7.22-7.35 (m, 4H), 8.31 (s, 2H), 8.90 (s, 1H), 11.11 (s, 1H) |
| 77 | 4 | 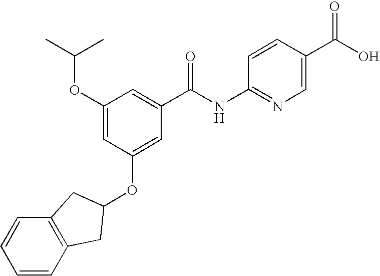 | 433 431 | δ_{H} (300MHz, DMSO-d₆) 1.26 (6H, d), 3.03 (2H, dd), 3.39 (2H, dd), 4.82 (1H, m), 5.34 (1H, m), 6.65 (1H, m), 7.13-7.20 (4H, br m), 7.27 (2H, m), 8.30 (2H, s), 9.87 (1H, s), 11.10 (1H, brs). |
| 78 | 18 | 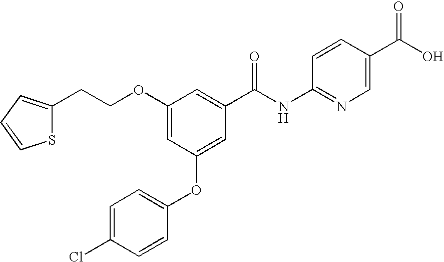 | 495/ 497 (MH)⁺for Cl isotopes | δ_{H} (300MHz, DMSO-d₆) 3.27 (2H, t), 4.30 (2H, t), 6.85 (1H, m), 6.98 (2H, m), 7.10 (2H, m), 7.22 (1H, m), 7.33 (1H, m), 7.46 (3H, m), 8.28 (2H, m), 8.88 (1H, s), 11.19 (1H, br s). |
| 79 | 4 | 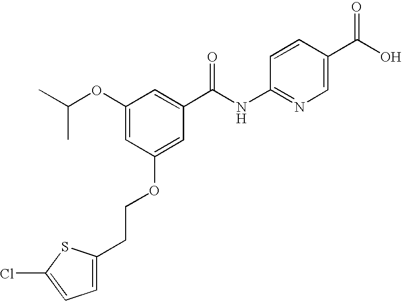 | 461.37 459.31 | δ_{H} ¹H NMR (d₆- DMSO): 1.27 (d, 6H), 3.20 (t, 2H), 4.23 (t, 2H), 4.71 (m, 1H), 6.67 (s, 1H), 6.85 (d, 1H), 6.95 (d, 1H), 7.19 (d, 2H), 8.29 (s, 2H), 8.87 (s, 1H), 11.10 (s, 1H) |
| 80 | 4 | 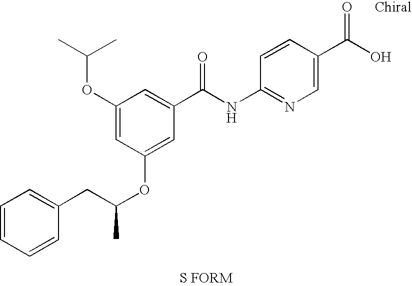 | 435 433 | δ_{H} (300MHz, DMSO-d₆) 1.22 (3H, d), 1.28 (6H, d), 2.83-3.03 (2H, br m), 4.67 (1H, m), 4.80 (1H, m), 6.62 (1H, s), 7.13-7.21 (3H, br m), 7.28 (4H, m), 8.30 (2H, s), 8.89 (1H, s), 11.08 (1H, br s). |
| 81 | 4 | 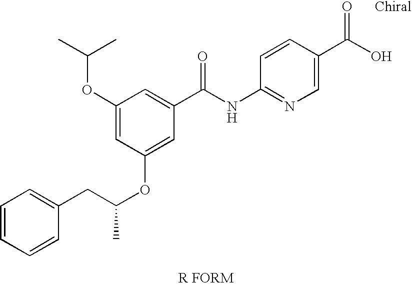 | 435 433 | δ_{H} (300MHz, DMSO-d₆) 1.23 (3H, d), 1.27 (6H, d), 2.83-3.02 (2H, br m), 4.67 (1H, m), 4.80 (1H, m) 6.61 (1H, s), 7.13-7.22 (3H, br m), 7.27 (4H, m), 8.29 (2H, s), 8.88 (1H, s), 11.08 (1H, br s). |

(a) The free phenol was alkylated alkylated as described in Routes 2 or 3 with methyl (3-bromomethyl) benzoate, and the resulting di- or tri-ester hydrolysed to the corresponding di- or tri-acid.

(b) The second alkyl group was introduced via a Mitsonobu reaction (see Alkylation Method C)

(c) The first alkyl group was introduced using sodium hydride as base and DMF as solvent.

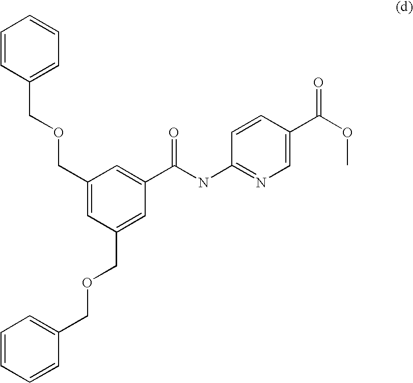

The requisite methyl ester starting material was prepared by a standard oxalyl chloride coupling of 3,5 dihydroxymethyl benzoic acid and the appropriate amine (see Example A); ¹H NMR 6 (d₆-DMSO): 3.88 (s, 3H) 4.58 (s, 2H) 4.62 (s, 2H) 7.24-7.42 (m, 10H) 7.6 (s, 1H) 7.95 (s, 2H) 8.35 (s, 2H) 8.91 (s, 1H) 11.22 (s, 1H M/Z 497 (M+H)⁺, 495 (M−H).

The requisite acid starting material was prepared by hydrolysis of the corresponding ester under standard conditions (see Example F):

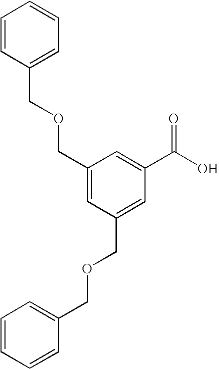

¹H NMR δ (d₆-DMSO): 4.62 (s, 2H) 4.68 (s, 2H) 7.32-7.46 (m, 10H) 7.64 (s, 1H) 7.92 (s, 2H) 13.05 (bs, 1H); m/z 380 (M+H)⁺.

The requisite ester starting material was prepared by alkylation of methyl 3,5 dihydroxymethyl benzoate using sodium hydride/THF and benzyl bromide (see Example F):

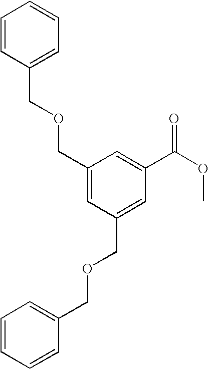

¹H NMR δ (d₆-DMSO): 3.85 (s, 3H) 4.54 (s, 2H) 4.6 (s, 2H) 7.24-7.39 (m, 10H) 7.59 (s, 1H) 7.85 (s, 2H); m/z 394 (M+NH4)⁺.

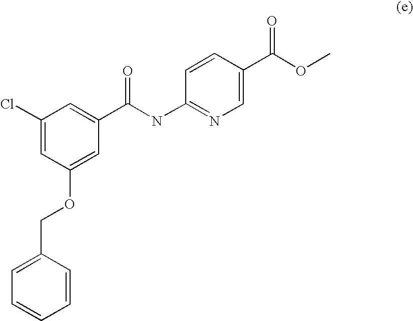

¹H NMR δ (d₆-DMSO): 3.86 (s, 3H), 5.22 (s, 2H), 7.30-7.49 (m, 6H), 7.63-7.69 (m, 2H), 8.28-8.36 (m, 2H), 8.90 (s, 1H); LCMS (ESI+) 397, 399 (MH⁺), (ESI−) 395, 397 (M−H).

The intermediate ester was prepared from commercially available starting materials as outlined below:

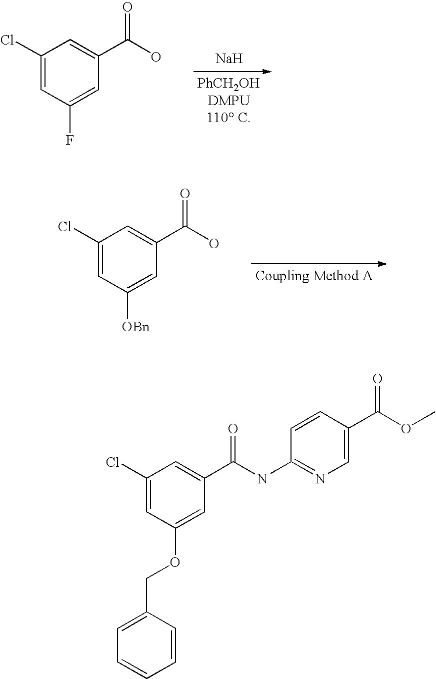

(f) The requisite methyl 2-[3-(2-fluorobenzyloxy)-5-hydroxymethyl] benzoyl amino-5-pyridine carboxylate starting material was prepared by a method analagous to that described in Example M:

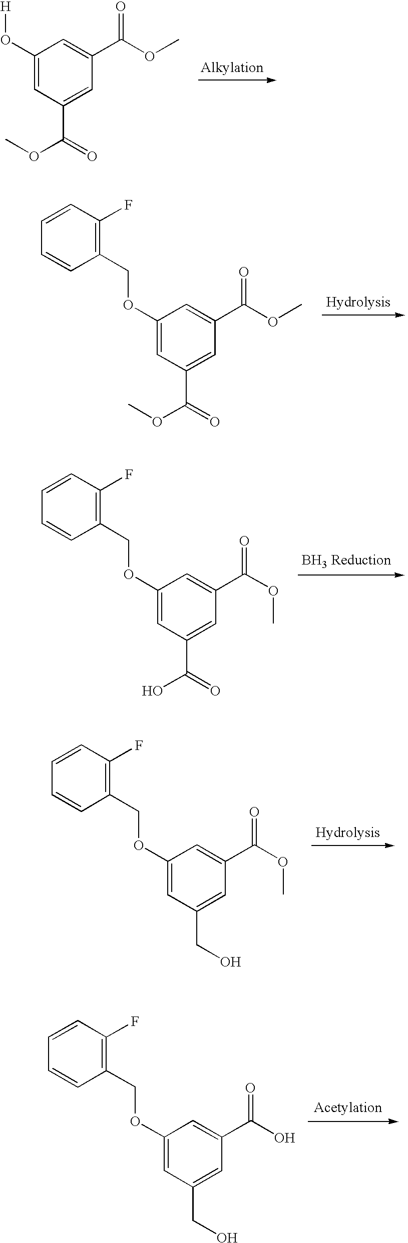

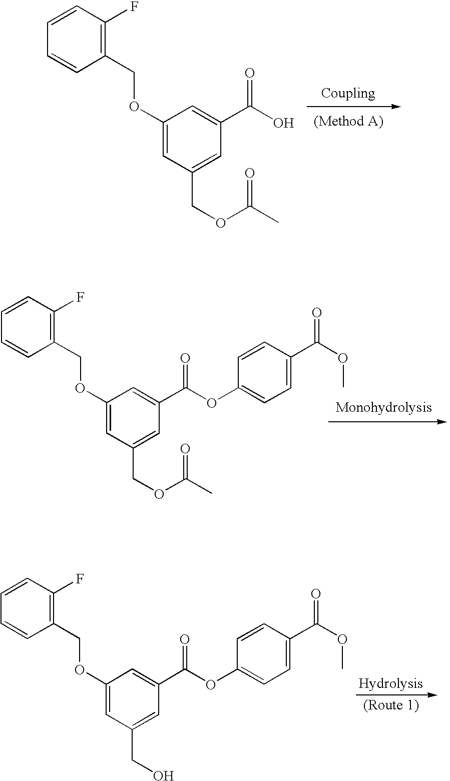

(g) Prepared by the method described in Example J (Mitsonobu reaction), starting from the methyl 2-[3-(2-fluorobenzyloxy)-5-hydroxymethyl] benzoyl amino-5-pyridine carboxylate intermediate (generic preparation described in footnote (f)).

(h) Generic Alkylation Method B was performed using the triflate of 2,2,2-trifluoroethanol as alkylating agent.

(i) The requisite methyl 3,5 di-[2-(2-thienyl) ethoxy] benzoate starting material was prepared in a manner essentially similar to that given in generic Alkylation Method A, using Mitsonobu alkylation conditions (triphenyl phosphine/DEAD).

(j) The requisite methyl 3-(Ar)alkyl-5-[2-(2-thienyl) ethoxy]benzoate starting material was prepared according to generic Alkylation Method C, starting from methyl 3-hydroxy-5-[2-(2-thienyl) ethoxy] benzoate which was prepared using Mitsonobu alkylation conditions (triphenyl phosphine/DEAD).

### EXAMPLE T—FURTHER EXAMPLES

The following table lists examples T₁ to T₁₀₅ which were made using analogous methods se described above. In this table:

(1) Route refers to method of preparation of final compound, as follows:

| | |
|---|---|
| Route 1 | see Example A; |
| Route 2 | see Example B; |
| Route 3 | see Example C; |
| Route 4 | see Example D; |
| Route 5. | see Example E; and |
| Route 6 | see Example F. |

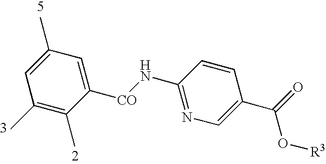

| No. | Route | 2 | 3 | 5 | MH+ | M − H |
|---|---|---|---|---|---|---|
| 1 | 1 | H | Benzyloxy | Benzyloxy | 455 | |
| 2 | 1 | H | Methoxy | β-Napthylmethoxy | 429 | |
| 3 | 1 | H | Methoxy | Isothiazol-4-ylmethoxy | 386 | 384 |
| 4 | 1 | H | (2-Methylbenzyl)oxy | (1- | 473 | |
| | | | | Methyl-Imidazol-2-yl)methoxy | | |
| 5 | 1 | H | Methoxy | (5-Methyl-Isoxazol-3-yl) | 384 | 382 |
| | | | | methoxy | | |
| 6 | 1 | H | CF₃ | CF₃ | 379 | 377 |
| 7 | 1 | H | Ethoxy | Ethoxy | | 329 |
| 8 | 1 | H | Methoxy | (2-Methylpyrid-3-yl)methyloxy | 394 | 392 |
| 9 | 1 | H | (2-Chlorobenzyl)oxy | (1-Methylpiperazin-4-yl) | | |
| | | | | methoxy | | |
| 10 | 1 | O- | H | Methylthio | 395 | 393 |
| | | Benzyl | | | | |
| 11 | 1 | Cl | H | Methylthio | 323 | 321 |
| 12 | 1 | I | H | I | 495 | 493 |
| 13 | 1 | Br | H | Isopropoxy | 379 | 377 |
| 14 | 1 | Cl | H | Cl | 311 | |
| 15 | 1 | Cl | H | I | 403 | |
| 16 | 1 | H | H | 2-Cyanophenoxy | | |
| 17 | 1 | H | H | 2-Chlorobenzyloxy | | |
| 18 | 1 | H | H | Phenoxy | 335 | 333 |
| 19 | 2 | H | (2-Difluoromethoxy)benzyloxy | (2-Difluoromethoxy)benzyloxy | 587 | 585 |
| 20 | 2 | H | 2,6-Dichloro-benzyloxy | (2,6-Dichloro)benzyloxy | | |
| 21 | 2 | H | 2-Chloro-5- | 2-Chloro-5- | 659 | 657 |
| | | | trifluoromethyl-benzyloxy | trifluoromethyl-benzyloxy | | |
| 22 | 2 | H | 2-Chloro-6-fluoro-benzyloxy | 2-Chloro-6-fluoro-benzyloxy | 559 | 557 |
| 23 | 2 | H | 2-Fluoro-5-trifluoromethyl- | 2-Fluoro-5-trifluoromethyl- | 627 | 625 |
| | | | benzyloxy | benzyloxy | | |
| 24 | 2 | H | 2-Trifluoromethyl-benzyloxy | 2-Trifluoromethyl-benzyloxy | 591 | 589 |
| 25 | 2 | H | 3-Chloro-2-fluoro-benzyloxy | 3-Chloro-2-fluoro-benzyloxy | 559 | 557 |
| 26 | 2 | H | 2,5-Difluoro-benzyloxy | 2,5-Difluoro-benzyloxy | 527 | 525 |
| 27 | 2 | H | 2-Cyano-benzyloxy | 2-Cyano-benzyloxy | 505 | 503 |
| 28 | 2 | H | 2,3-Difluoro-benzyloxy | 2,3-Difluoro-benzyloxy | 527 | 525 |
| 29 | 2 | H | 3-Cyano-benzyloxy | 3-Cyano-benzyloxy | | 503 |
| 30 | 2 | H | (2-Methylpyrid-3-yl)methoxy | (2-Methylpyrid-3-yl)methoxy | 485 | 483 |
| 31 | 2 | H | (5-Methyl-Isoxazol-3-yl) | (5-MethylIsoxazol-3-yl) | 465 | 463 |
| | | | methoxy | methoxy | | |
| 32 | 2 | H | 4-Carboxybenzyloxy | 4-Carboxybenzyloxy | | 541 |
| 33 | 2 | H | (1,2,5-Thiadiazol-3-yl)methoxy | (1,2,5-Thiadiazol-3-yl)methoxy | | 469 |
| 34 | 2 | H | 2-Chlorobenzyloxy | 2-Chlorobenzyloxy | 523 | |
| 35 | 2 | H | 2-Bromobenzyloxy | 2-Bromobenzyloxy | | |
| 36 | 2 | H | 2-Methylbenzyloxy | 2-Methylbenzyloxy | 483 | 481 |
| 37 | 2 | H | 2-Fluorobenzyloxy | 2-Fluorobenzyloxy | 491 | 489 |
| 38 | 2 | H | 3-Chlorobenzyloxy | 3-Chlorobenzyloxy | 523 | |
| 39 | 2 | H | 3-Methoxybenzyloxy | 3-Methoxybenzyloxy | | |
| 40 | 2 | H | 3-carboxybenzyloxy | 3-carboxybenzyloxy | | |
| 41 | 3 | H | OH | Benzyloxy | 365 | 363 |
| 42 | 3 | H | 2-Bromobenzyloxy | 2-Cyanobenzyloxy | 558 | |
| 43 | 3 | H | 2-Chlorobenzyloxy | 2-Cyanobenzyloxy | 514 | |
| 44 | 3 | H | 2-Methylbenzyloxy | 2-Cyanobenzyloxy | 494 | 492 |
| 45 | 3 | H | 2-Nitrobenzyloxy | 2-Cyanobenzyloxy | 525 | 523 |
| 46 | 3 | H | 3-Fluoro-6-methyl-benzyloxy | 2-Cyanobenzyloxy | 512 | 510 |
| 47 | 3 | H | 2-Trifluoromethyl-benzyloxy | 2-Cyanobenzyloxy | 548 | 546 |
| 48 | 3 | H | 2,6-Difluoro-benzyloxy | 2-Cyanobenzyloxy | 516 | |
| 49 | 3 | H | 2-Fluorobenzyloxy | 2-Cyanobenzyloxy | 498 | 496 |
| 50 | 3 | H | 2-Iodobenzyloxy | Benzyloxy | 581 | 579 |
| 51 | 3 | H | 2-Bromo-5-fluoro-benzyloxy | 2-Cyanobenzyloxy | | |
| 52 | 3 | H | 2-Chloro-6-fluoro-3- | Benzyloxy | 521 | 519 |
| | | | methyl-benzyloxy | | | |
| 53 | 3 | H | 3-Fluoro-6-methyl-benzyloxy | Benzyloxy | 487 | 485 |
| 54 | 3 | H | 2,5-Difluoro-benzyloxy | 2-Cyanobenzyloxy | 516 | 514 |
| 55 | 3 | H | 2-Cyanobenzyloxy | Benzyloxy | 480 | 478 |
| 56 | 3 | H | 2-Bromo-benzyloxy | Benzyloxy | 533 | |
| 57 | 3 | H | 2,5-Dichloro-benzyloxy | Benzyloxy | 523 | |
| 58 | 3 | H | (5-MethylIsoxazol-3-yl) | 2-Methylbenzyloxy | 474 | 472 |
| | | | methoxy | | | |
| 59 | 3 | H | 2,6-Difluoro-benzyloxy | Benzyloxy | 491 | |
| 60 | 3 | H | 3-Methoxybenzyloxy | 2-Cyanobenzyloxy | 510 | |
| 61 | 3 | H | Pyrid-2-ylmethoxy | 2-Methylbenzyloxy | 470 | 468 |
| 62 | 3 | H | 3-Methylbenzyloxy | 2-Cyanobenzyloxy | 494 | 492 |
| 63 | 3 | H | (2-Methylthiazol-4-yl)methoxy | 2-Methylbenzyloxy | 490 | 488 |
| 64 | 3 | H | (1S)-phenylethoxy | Benzyloxy | 469 | 467 |
| 65 | 3 | H | 2-(4-Methylthiazol-yl)ethoxy | 2-Methylbenzyloxy | | |
| 66 | 3 | H | 3-Chlorobenzyloxy | 2-Cyanobenzyloxy | 514 | |
| 67 | 3 | H | Cyclopentyloxy | Benzyloxy | 433 | 431 |
| 68 | 3 | H | 3-carboxybenzyloxy | 2-Cyanobenzyloxy | 524 | |
| 69 | 3 | H | 2-Carboxybenzyloxy | 2-Cyanobenzyloxy | 524 | |
| 70 | 3 | H | Cyclohexyloxy | Benzyloxy | 461 | 459 |
| 71 | 3 | H | 3-Cyanobenzyloxy | 2-Cyanobenzyloxy | 505 | |
| 72 | 3 | H | n-Propoxy | Benzyloxy | 407 | 405 |
| 73 | 3 | H | (1R)Phenylethoxy | Benzyloxy | 469 | 467 |
| 74 | 3 | H | 2,3,5-Trifluorobenzyloxy | Benzyloxy | 509 | 507 |
| 75 | 3 | H | 2-Pheny-Ibenzyloxy | Benzyloxy | 531 | 529 |
| 76 | 3 | H | Allyloxy | Benzyloxy | 405 | 403 |
| 77 | 3 | H | (2-Methylpyrid-3-yl)methoxy | 2-Methylbenzyloxy | 484 | 482 |
| 78 | 3 | H | Thiazol-4-ylmethoxy | 2-Methylbenzyloxy | | |
| 79 | 3 | H | Pyrid-3-ylmethoxy | 2-Methylbenzyloxy | | |
| 80 | 3 | H | (6-Methylpyrid-2-yl)methoxy | 2-Methylbenzyloxy | | |
| 81 | 3 | H | (5-MethylIsoxazol-3-yl) | Benzyloxy | 460 | |
| | | | methoxy | | | |
| 82 | 3 | H | 2-Methyl-3-trifluoromethyl- | 2-Cyanobenzyloxy | 562 | 560 |
| | | | benzyloxy | | | |
| 83 | 3 | H | Isopropoxy | Benzyloxy | 407 | 405 |
| 84 | 3 | H | Cyclopropylmethoxy | Benzyloxy | 419 | 417 |
| 85 | 3 | H | 2-(Phenylsulphonylmethyl) | 2-Cyanobenzyloxy | 634 | |
| | | | benzyloxy | | | |
| 86 | 3 | H | 2-(Pyrid-2-yl)ethoxy | 2-Methylbenzyloxy | 484 | |
| 87 | 3 | H | Methoxy | Benzyloxy | 379 | 377 |
| 88 | 3 | H | OH | 2-Cyanobenzyloxy | 390 | 388 |
| 89 | 3 | H | 2-(N-morpholino)ethoxy | 2-Cyanobenzyloxy | 503 | |
| 90 | 3 | H | (1- | Benzyloxy | 462 | 460 |
| | | | Methylpiperazin-4-yl)methoxy | | | |
| 91 | 3 | H | 2-(N-morpholino)ethoxy | Benzyloxy | 478 | |
| 92 | 3 | H | 2-(Pyrid-4-yl)ethoxy | 2-Methylbenzyloxy | 484 | 482 |
| 93 | 3 | H | (4,6-Dimethoxypyrimid-2-yl) | 2-Methylbenzyloxy | 531 | 529 |
| | | | methoxy | | | |
| 94 | 3 | H | Carboxymethoxy | 2-Methylbenzyloxy | 437 | 435 |
| 95 | 4 | H | Isopropoxy | 2-(4-Methylthiazol-5-yl)ethoxy | 442 | 440 |
| 96 | 4 | H | Isopropoxy | 2-Methylbenzyloxy | 421 | 419 |
| 97 | 4 | H | Isopropoxy | (5-Methyl-Isoxazol-3-yl) | 412 | 410 |
| | | | | methoxy | | |
| 98 | 4 | H | Isopropoxy | Isobutoxy | 373 | 371 |
| 99 | 5 | H | 2-Methylbenzoylamino | 2-Methylbenzoylamino | 509 | |
| 100 | 6 | H | Phenoxymethyl | Phenoxymethyl | 455 | |
| 101 | 5 | H | Acetoxy | (2-Methyl)benzyloxy | 435 | |
| 102 | 5 | H | H | (2-Chloro)benzyloxy | | |
| 103 | 5 | H | 2-Difluoromethoxy-Benzyloxy | 2-Difluoromethoxy-Benzyloxy | 601 | |
| 104 | 5 | H | 2-Methylbenzyloxy | 2-Cyanobenzyloxy | 508 | 506 |
| 105 | 5 | H | 2-(N-morpholino)ethoxy | Benzyloxy | 492 | |

In Examples 1-100 R³ is H; in Examples 101-105 R³ is methyl.

Biological

Tests:

The biological effects of the compounds of the invention may be tested in the following way:

(1) Enzymatic activity of GLK may be measured by incubating GLK, ATP and glucose. The rate of product formation may be determined by coupling the assay to a G-6-P dehydrogenase, NADP/NADPH system and measuring the increase in optical density at 340 nm (Matschinsky et al 1993).

(2) A GLK/GLKRP binding assay for measuring the binding interactions between

GLK and GLKRP. The method may be used to identify compounds which modulate GLK by modulating the interaction between GLK and GLKRP. GLKRP and GLK are incubated with an inhibitory concentration of F-6-P, optionally in the presence of test compound, and the extent of interaction between GLK and GLKRP is measured. Compounds which either displace F-6-P or in some other way reduce the GLK/GLKRP interaction will be detected by a decrease in the amount of GLK/GLKRP complex formed. Compounds which promote F-6-P binding or in some other way enhance the GLK/GLKRP interaction will be detected by an increase in the amount of GLK/GLKRP complex formed. A specific example of such a binding assay is described below

GLK/GLKRP Scintillation Proximity Assay

Recombinant human GLK and GLKRP were used to develop a “mix and measure” 96 well SPA (scintillation proximity assay). (A schematic representation of the assay is given in FIG. 3). GLK (Biotinylated) and GLKRP are incubated with streptavidin linked SPA beads (Amersham) in the presence of an inhibitory concentration of radiolabelled [3H]F-6-P (Amersham Custom Synthesis TRQ8689), giving a signal as depicted in FIG. 3. Compounds which either displace the F-6-P or in some other way disrupt the GLK/GLKRP binding interaction will cause this signal to be lost.

Binding assays were performed at room temperature for 2 hours. The reaction mixtures contained 50 mM Tris-HCl (pH=7.5), 2 mM ATP, 5 mM MgCl₂, 0.5 mM DTT, recombinant biotinylated GLK (0.1 mg), recombinant GLKRP (0.1 mg), 0.05 mCi [3H] F-6-P (Amersham) to give a final volume of 100 ml. Following incubation, the extent of GLK/GLKRP complex formation was determined by addition of 0.1 mg/well avidin linked SPA beads (Amersham) and scintillation counting on a Packard TopCount NXT.

The exemplified compounds described above were found to have an activity of at least 40% activity at 10 μm when tested in the GLK/GLKRP scintillation proximity assay.

(3) A F-6-P/GLKRP binding assay for measuring the binding interaction between GLKRP and F-6-P. This method may be used to provide further information on the mechanism of action of the compounds. Compounds identified in the GLK/GLKRP binding assay may modulate the interaction of GLK and GLKRP either by displacing F-6-P or by modifying the GLK/GLKRP interaction in some other way. For example, protein-protein interactions are generally known to occur by interactions through multiple binding sites. It is thus possible that a compound which modifies the interaction between GLK and GLKRP could act by binding to one or more of several different binding sites.

The F-6-P/GLKRP binding assay identifies only those compounds which modulate the interaction of GLK and GLKRP by displacing F-6-P from its binding site on GLKRP.

GLKRP is incubated with test compound and an inhibitory concentration of F-6-P, in the absence of GLK, and the extent of interaction between F-6-P and GLKRP is measured. Compounds which displace the binding of F-6-P to GLKRP may be detected by a change in the amount of GLKRP/F-6-P complex formed. A specific example of such a binding assay is described below

F-6-P/GLKRP Scintillation Proximity Assay

Recombinant human GLKRP was used to develop a “mix and measure” 96 well scintillation proximity assay. (A schematic representation of the assay is given in FIG. 4). FLAG-tagged GLKRP is incubated with protein A coated SPA beads (Amersham) and an anti-FLAG antibody in the presence of an inhibitory concentration of radiolabelled [3H]F-6-P. A signal is generated as depicted in FIG. 4. Compounds which displace the F-6-P will cause this signal to be lost. A combination of this assay and the GLK/GLKRP binding assay will allow the observer to identify compounds which disrupt the GLK/GLKRP binding interaction by displacing F-6-P.

Binding assays were performed at room temperature for 2 hours. The reaction mixtures contained 50 mM Tris-HCl (pH=7.5), 2 mM ATP, 5 mM MgCl₂, 0.5 mM DTT, recombinant FLAG tagged GLKRP (0.1 mg), Anti-Flag M2 Antibody (0.2 mg) (IBI Kodak), 0.05 mCi [3H] F-6-P (Amersham) to give a final volume of 100 ml. Following incubation, the extent of F-6-P/GLKRP complex formation was determined by addition of 0.1 mg/well protein A linked SPA beads (Amersham) and scintillation counting on a Packard TopCount NXT.

Production of Recombinant GLK and GLKRP:

Preparation of mRNA

Human liver total mRNA was prepared by polytron homogenisation in 4M guanidine isothiocyanate, 2.5 mM citrate, 0.5% Sarkosyl, 100 mM b-mercaptoethanol, followed by centrifugation through 5.7M CsCl, 25 mM sodium acetate at 135,000 g (max) as described in Sambrook J, Fritsch EF & Maniatis T, 1989.

Poly A⁺ mRNA was prepared directly using a FastTrack™ RNA isolation kit (Invitrogen).

PCR Amplification of GLK and GLKRP cDNA Sequences

Human GLK and GLKRP cDNA was obtained by PCR from human hepatic mRNA using established techniques described in Sambrook, Fritsch & Maniatis, 1989. PCR primers were designed according to the GLK and GLKRP cDNA sequences shown in Tanizawa et al 1991 and Bonthron, D. T. et al 1994 (later corrected in Warner, J. P. 1995).

Cloning in Bluescript II Vectors

GLK and GLKRP cDNA was cloned in E. coli using pBluescript II, (Short et al 1998) a recombinant cloning vector system similar to that employed by Yanisch-Perron C et al (1985), comprising a colEI-based replicon bearing a polylinker DNA fragment containing multiple unique restriction sites, flanked by bacteriophage T3 and T7 promoter sequences; a filamentous phage origin of replication and an ampicillin drug resistance marker gene.

Transformations

E. Coli transformations were generally carried out by electroporation. 400 ml cultures of strains DH5a or BL21(DE3) were grown in L-broth to an OD 600 of 0.5 and harvested by centrifugation at 2,000 g. The cells were washed twice in ice-cold deionised water, resuspended in 1 ml 10% glycerol and stored in aliquots at −70° C. Ligation mixes were desalted using Millipore V series™ membranes (0.0025 mm) pore size). 40 ml of cells were incubated with 1 ml of ligation mix or plasmid DNA on ice for 10 minutes in 0.2 cm electroporation cuvettes, and then pulsed using a Gene Pulser™ apparatus (BioRad) at 0.5 kVcm⁻¹, 250 mF, 250 ?. Transformants were selected on L-agar supplemented with tetracyline at 10 mg/ml or ampicillin at 100 mg/rml.

Expression

GLK was expressed from the vector pTB375NBSE in E. coli BL21 cells, producing a recombinant protein containing a 6-His tag immediately adjacent to the N-terminal methionine. Alternatively, another suitable vector is pET21(+)DNA. Novagen, Cat number 697703. The 6-His tag was used to allow purification of the recombinant protein on a column packed with nickel-nitrilotriacetic acid agarose purchased from Qiagen (cat no 30250).

GLKRP was expressed from the vector pFLAG CTC (IBI Kodak) in E. coli BL21 cells, producing a recombinant protein containing a C-terminal FLAG tag. The protein was purified initially by DEAE Sepharose ion exchange followed by utilisation of the FLAG tag for final purification on an M2 anti-FLAG immunoaffinity column purchased from Sigma-Aldrich (cat no. A1205).

Biotinylation of GLK:

GLK was biotinylated by reaction with biotinamidocaproate N-hydroxysuccinimide ester (biotin-NHS) purchased from Sigma-Aldrich (cat no. B2643). Briefly, free amino groups of the target protein (GLK) are reacted with biotin-NHS at a defined molar ratio forming stable amide bonds resulting in a product containing covalently bound biotin. Excess, non-conjugated biotin-NHS is removed from the product by dialysis. Specifically, 7.5 mg of GLK was added to 0.31 mg of biotin-NHS in mL of 25 mM HEPES pH=7.3, 0.1.5M KCl, 1 mM dithiothreitol, 1 mM EDTA, 1 mM MgCl₂ (buffer A). This reaction mixture was dialysed against 100 mL of buffer A containing a further 22 mg of biotin-NHS. After 4 hours excess biotin-NHS was removed by extensive dialysis against buffer A.

Pharmaceutical Compositions

The following illustrate representative pharmaceutical dosage forms of the invention as defined herein (the active ingredient being termed “Compound X”), for therapeutic or prophylactic use in humans:

| (a) | Tablet I | mg/tablet |
|---|---|---|
| | Compound X | 100 |
| | Lactose Ph.Eur | 182.75 |
| | Croscarmellose sodium | 12.0 |
| | Maize starch paste (5% w/v paste) | 2.25 |
| | Magnesium stearate | 3.0 |
| (b) | Tablet II | mg/tablet |
| | Compound X | 50 |
| | Lactose Ph.Eur | 223.75 |
| | Croscarmellose sodium | 6.0 |
| | Maize starch | 15.0 |
| | Polyvinylpyrrolidone (5% w/v paste) | 2.25 |
| | Magnesium stearate | 3.0 |
| (c) | Tablet III | mg/tablet |
| | Compound X | 1.0 |
| | Lactose Ph.Eur | 93.25 |
| | Croscarmellose sodium | 4.0 |
| | Maize starch paste (5% w/v paste) | 0.75 |
| | Magnesium stearate | 1.0 |
| (d) | Capsule | mg/capsule |
| | Compound X | 10 |
| | Lactose Ph.Eur | 488.5 |
| | Magnesium | 1.5 |
| (e) | Injection I | (50 mg/ml) |
| | Compound X | 5.0% w/v |
| | 1 M Sodium hydroxide solution | 15.0% v/v |
| | 0.1 M Hydrochloric acid (to adjust pH = to 7.6) | |
| | Polyethylene glycol 400 | 4.5% w/v |
| | Water for injection to 100% | |
| (f) | Injection II | (10 mg/ml) |
| | Compound X | 1.0% w/v |
| | Sodium phosphate BP | 3.6% w/v |
| | 0.1 M Sodium hydroxide solution | 15.0% v/v |
| | Water for injection to 100% | |
| | | (1 mg/ml, |
| (g) | Injection III | buffered to pH = 6) |
| | Compound X | 0.1% w/v |
| | Sodium phosphate BP | 2.26% w/v |
| | Citric acid | 0.38% w/v |
| | Polyethylene glycol 400 | 3.5% w/v |
| | Water for injection to 100% | |
| (h) | Aerosol I | mg/ml |
| | Compound X | 10.0 |
| | Sorbitan trioleate | 13.5 |
| | Trichlorofluoromethane | 910.0 |
| | Dichlorodifluoromethane | 490.0 |
| (i) | Aerosol II | mg/ml |
| | Compound X | 0.2 |
| | Sorbitan trioleate | 0.27 |
| | Trichlorofluoromethane | 70.0 |
| | Dichlorodifluoromethane | 280.0 |
| | Dichlorotetrafluoroethane | 1094.0 |
| (j) | Aerosol III | mg/ml |
| | Compound X | 2.5 |
| | Sorbitan trioleate | 3.38 |
| | Trichlorofluoromethane | 67.5 |
| | Dichlorodifluoromethane | 1086.0 |
| | Dichlorotetrafluoroethane | 191.6 |
| (k) | Aerosol IV | mg/ml |
| | Compound X | 2.5 |
| | Soya lecithin | 2.7 |
| | Trichlorofluoromethane | 67.5 |
| | Dichlorodifluoromethane | 1086.0 |
| | Dichlorotetrafluoroethane | 191.6 |
| (l) | Ointment | ml |
| | Compound X | 40 mg |
| | Ethanol | 300 μl |
| | Water | 300 μl |
| | 1-Dodecylazacycloheptan-2-one | 50 μl |
| | Propylene glycol | to 1 ml |

Note

The above formulations may be obtained by conventional procedures well known in the pharmaceutical art. The tablets (a)-(c) may be enteric coated by conventional means, for example to provide a coating of cellulose acetate phthalate. The aerosol formulations (h)-(k) may be used in conjunction with standard, metered dose aerosol dispensers, and the suspending agents sorbitan trioleate and soya lecithin may be replaced by an alternative suspending agent such as sorbitan monooleate, sorbitan sesquioleate, polysorbate 80, polyglycerol oleate or oleic acid.

### REFERENCES

1 Printz, R. L., Magnuson, M. A. and Granner, D. K. (1993) Annual Review of Nutrition 13, 463-96

2 DeFronzo, R. A. (1988) Diabetes 37, 667-87

3 Froguel, P., Zouali, H., Vionnet, N., Velho, G., Vaxillaire, M., Sun, F., Lesage, S., Stoffel, M., Takeda, J. and Passa, P. (1993) New England Journal of Medicine 328, 697-702

4 Bell, G. I., Pilkis, S. J., Weber, I. T. and Polonsky, K. S. (1996) Annual Review of Physiology 58, 171-86

5 Velho, G., Petersen, K. F., Perseghin, G., Hwang, J. H., Rothman, D. L., Pueyo, M. E., Cline, G. W., Froguel, P. and Shulman, G. I. (1996) Journal of Clinical Investigation 98, 1755-61

6 Christesen, H. B., Jacobsen, B. B., Odili, S., Buettger, C., Cuesta-Munoz, A., Hansen, T., Brusgaard, K., Massa, O., Magnuson, M. A., Shiota, C., Matschinsky, F. M. and Barbetti, F. (2002) Diabetes 51, 1240-6

7 Glaser, B., Kesavan, P., Heyrnan, M., Davis, E., Cuesta, A., Buchs, A., Stanley, C. A., Thornton, P. S., Perrnutt, M. A., Matschinsky, F. M. and Herold, K. C. (1998) New England Journal of Medicine 338, 226-30

8 Caro, J. F., Triester, S., Patel, V. K., Tapscott, E. B., Frazier, N. L. and Dohm, G. L. (1995) Hormone & Metabolic Research 27, 19-22

9 Desai, U. J., Slosberg, E. D., Boettcher, B. R., Caplan, S. L., Fanelli, B., Stephan, Z., Gunther, V. J., Kaleko, M. and Connelly, S. (2001) Diabetes 50, 2287-95

10 Shiota, M., Postic, C., Fujimoto, Y., Jetton, T. L., Dixon, K., Pan, D., Grimsby, J., Grippo, J. F., Magnuson, M. A. and Cherrington, A. D. (2001) Diabetes 50, 622-9

11 Ferre, T., Pujol, A., Riu, E., Bosch, F. and Valera, A. (1996) Proceedings of the National Academy of Sciences of the United States of America 93, 7225-30

12 Seoane, J., Barbera, A., Telemaque-Potts, S., Newgard, C. B. and Guinovart, J. J. (1999) Journal of Biological Chemistry 274, 31833-8

13 Moore, M. C., Davis, S. N., Mann, S. L. and Cherrington, A. D.,(2001) Diabetes Care 24, 1882-7

14 Alvarez, E., Roncero, I., Chowen, J. A., Vazquez, P. and Blazquez, E. (2002) Journal of Neurochemistry 80, 45-53

15 Lynch, R. M., Tompkins, L. S., Brooks, H. L., Dunn-Meynell, A. A. and Levin, B. E. (2000) Diabetes 49, 693-700

16 Roncero, I., Alvarez, E., Vazquez, P. and Blazquez, E. (2000) Journal of Neurochemistry 74, 1848-57

17 Yang, X. J., Kow, L. M., Funabashi, T. and Mobbs, C. V. (1999) Diabetes 48, 1763-1772

18 Schuit, F. C., Huypens, P., Heimberg, H. and Pipeleers, D. G. (2001) Diabetes 50, 1-11

19 Levin, B. E. (2001) International Journal of Obesity 25

20 Alvarez, E., Roncero, I., Chowen, J. A., Thorens, B. and Blazquez, E. (1996) Journal of Neurochemistry 66, 920-7

21 Mobbs, C. V., Kow, L. M. and Yang, X. J. (2001) American Journal of Physiology—Endocrinology & Metabolism 281, E649-54

22 Levin, B. E., Dunn-Meynell, A. A. and Routh, V. H. (1999) American Journal of Physiology 276, R1223-31

23 Spanswick, D., Smith, M. A., Groppi, V. E., Logan, S. D. and Ashford, M. L. (1997) Nature 390, 521-5

24 Spanswick, D., Smith, M. A., Mirshamsi, S., Routh, V. H. and Ashford, M. L. (2000) Nature Neuroscience 3, 757-8

25 Levin, B. E. and Dunn-Meynell, A. A. (1997) Brain Research 776, 146-53

26 Levin, B. E., Govek, E. K. and Dunn-Meynell, A. A. (1998) Brain Research 808, 317-9

27 Levin, B. E., Brown, K. L. and Dunn-Meynell, A. A. (1996) Brain Research 739, 293-300

28 Rowe, I. C., Boden, P. R. and Ashford, M. L. (1996) Journal of Physiology 497, 365-77

29 Fujimoto, K., Sakata, T., Arase, K., Kurata, K., Okabe, Y. and Shiraishi, T. (1985) Life Sciences 37, 2475-82

30 Kurata, K., Fujimoto, K. and Sakata, T. (1989) Metabolism: Clinical & Experimental 38, 46-51

31 Kurata, K., Fujimoto, K., Sakata, T., Etou, H. and Fukagawa, K. (1986) Physiology & Behavior 37, 615-20

1. A method for the treatment or prevention of a disease or medical condition mediated through GLK, comprising administering a compound of Formula (I) or a salt, solvate, or prodrug thereof, 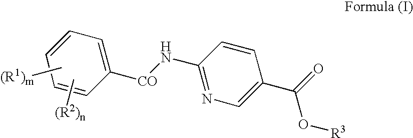
wherein
each R¹ is independently selected from OH, —(CH₂)₁₋₄H, —CH₃₋ₐFₐ, —(CH₂)₁₋₄CH₃₋ₐFₐ, halo, C₁₋₆ alkyl, C₂₋₆ alkenyl, C₂₋₆ alkynyl, NO₂, NH₂, —NH—C₁₋₄ alkyl, —N-di-(C₁₋₄ alkyl), CN, or formyl;
each R² is the group Y—X—;
R³ is selected from hydrogen or C₁₋₆ alkyl;
each R⁴ is independently selected from halo, —CH₃₋ₐFₐ, CN, NO₂, NH₂, C₁₋₆ alkyl, —OC₁₋₆ alkyl, —COOH, —C(O)OC₁₋₆ alkyl, OH, phenyl, or R⁵—X¹;
R⁵ is selected from hydrogen, C₁₋₆ alkyl, —CH₃₋ₐFₐ, phenyl, naphthyl, heterocyclyl, and C₃₋₇ cycloalkyl, and is optionally substituted with halo, C₁₋₆ alkyl, —CH₃₋ₐFₐ, CN, NO₂, NH₂, COOH, or —C(O)OC₁₋₆ alkyl, and each phenyl, naphthyl or heterocyclyl ring in R⁵ is optionally substituted with halo, CH₃₋ₐFₐ, CN, NO₂, NH₂, C₁₋₆ alkyl, —OC₁₋₆ alkyl, COOH, —C(O)OC₁₋₆ alkyl, or OH;
R⁶ is independently selected from hydrogen, C₁₋₆ alkyl or —C₂₋₄ alkyl-O—C₁₋₄ alkyl;
each X and X¹ is a linker independently selected from —O-Z-, —O-Z-O-Z-, —C(O)O-Z-, —OC(O)-Z-, —S-Z-, —SO-Z-, —SO₂-Z-, —N(R⁶)-Z-, —N(R⁶)SO₂-Z-, —SO₂N(R⁶)-Z-, —(CH₂)₁₋₄—, —CH═CH-Z-, —C≡C-Z-, —N(R⁶)CO-Z-, —CON(R⁶)-Z-, —C(O)N(R⁶)S(O)₂-Z-, —S(O)₂N(R⁶)C(O)-Z-, —C(O)-Z-, or a direct bond;
each Y is independently selected from aryl-Z¹-, heterocyclyl-Z¹-, C₃₋₇ cycloalkyl-Z¹-, C₁₋₆ alkyl, C₂₋₆ alkenyl, C₂₋₆ alkynyl, or —(CH₂)₁₋₄CH₃₋ₐFₐ, wherein each Y is independently optionally substituted with up to three R⁴ groups;
each Z¹ is independently a direct bond or a group of the formula —(CH₂)ₚ—C(R⁶)₂—(CH₂)_{q}—;
each Z¹ is independently a direct bond or a group of the formula —(CH₂)ₚ—C(R⁶)₂—(CH₂)_{q}—;
each a is independently 1, 2, or 3;
m is 0, 1, or 2;
n is 0, 1, 2, 3, or 4;
and n+m>0;
p is an integer between 0 and 2;
q is an integer between 0 and 2; and
p+q<4.

2. A method of claim 1 wherein the compound is administered together with a pharmaceutically acceptable diluent or carrier.

3. A compound of Formula (Ib) or a salt, solvate or prodrug thereof 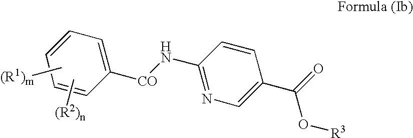
wherein
each R¹ is independently selected from OH, —(CH₂)₁₋₄OH, —CH₃₋ₐFₐ, —(CH₂)₁₋₄CH₃₋ₐFₐ, halo, C₁₋₆ alkyl, C₂₋₆ alkenyl, C₂₋₆ alkynyl, NO₂, NH₂, —NH—C₁₋₄ alkyl, —N-di-(C₁₋₄ alkyl), CN, or formyl;
each R² is the group Y—X—;
R³ is selected from hydrogen or C₁₋₆ alkyl;
each R⁴ is independently selected from halo, —CH₃₋ₐFₐ, CN, NO₂, NH₂, C₁₋₆ alkyl, —OC₁₋₆alkyl, —COOH, —C(O)OC₁₋₆alkyl, OH, phenyl, or R⁵—X;
R⁵ is selected from hydrogen, C₁₋₆ alkyl, —CH₃₋ₐFₐ, phenyl, naphthyl, heterocyclyl, or C₃₋₇ cycloalkyl, and is optionally substituted with halo, C₁₋₆alkyl, —CH₃₋ₐFₐ, CN, NO₂, NH₂, COOH or —C(O)OC₁₋₆ alkyl, and each phenyl, naphthyl or heterocyclyl ring in R⁵ is optionally substituted with halo, CH₃₋ₐFₐ, CN, NO₂, NH₂, C₁₋₆ alkyl, —OC₁₋₆alkyl, COOH, —C(O)OC₁₋₆alkyl, or OH;
R⁶ is independently selected from hydrogen, C₁₋₆alkyl or —C₂₋₄ alkyl-O—C₁₋₄ alkyl;
each X and X¹ is a linker independently selected from —O-Z-, —C(O)O-Z-, —OC(O)-Z-, —S-Z-, —SO-Z-, —SO₂-Z-, —N(R⁶)-Z-, —N(R⁶)SO₂-Z-, —SO₂N(R⁶)-Z-, —(CH₂)₁₋₄—, —CH═CH-Z-, —C≡C-Z-, —N(R⁶)CO-Z-, —CON(R⁶)-Z-, —C(O)N(R⁶)S(O)₂-Z-, —S(O)₂N(R⁶)C(O)-Z-, —C(O)-Z-, or a direct bond;
each Y is independently selected from aryl-Z¹-, heterocyclyl-Z¹-, C₃₋₇cycloalkyl-Z¹-, C₁₋₆alkyl, C₂₋₆alkenyl, C₂₋₆alkynyl, or —(CH₂)₁₋₄CH₃₋ₐFₐ, wherein each Y is independently optionally substituted with up to three R⁴ groups;
each Z is independently a direct bond or a group of the formula —(CH₂)ₚ—C(R⁶)₂—(CH₂)_{q}—;
each Z¹ is independently a direct bond or a group of the formula —(CH₂)ₚ—C(R⁶)₂—(CH₂)_{q}—;
each a is independently 1, 2, or 3;
m is 0, 1, or 2;
n is 0, 1, 2, 3, or 4;
and n+m>0;
p is an integer between 0 and 2;
q is an integer between 0 and 2; and
p+q<4.
with the proviso that
(i) when R³ is hydrogen or methyl, m is 1 and n is 0, then R¹ cannot be 2-halo or 2-methyl;
(ii) when R³ is hydrogen or methyl, m is 2 and n is 0, then (R¹)ₘ is other than di-C₁₋₄ alkyl, di-halo, or mono-halo-mono-C₁₋₄ alkyl;
(iii) when R³ is hydrogen, methyl, or ethyl, m is 0, n is 1, R² is a substituent at the 2-position or 4-position, and X is —O— or a direct bond, then Y cannot be methyl, phenyl, or benzyl, and R⁴ (when present) cannot be methyl or trifluoromethyl;
(iv) when R³ is hydrogen, m is 0, n is 2, and X is a direct bond, then (R²)ₘ is other than 2,4-diphenyl;
(v) when R³ is hydrogen, m is 0 and n is 3, then at least one R² must be other than methoxy; and
(vi) the following compound is excluded: ethyl 6-[(3-tert-butyl-2-hydroxy-6-methyl-5-nitrobenzoyl)amino]nicotinate.

4. A compound according to claim 3, wherein m is 0 or 1 and n is 1 or 2.

5. A compound according to claim 4, wherein n+m is 2 and the R¹ and/or R² groups are substituents at the 3- and 5-positions.

6. A compound according to claim 3, wherein each R¹ is independently selected from OH, —CH₃₋ₐFₐ, halo, C₁₋₄ alkyl, or CN.

7. A compound according to claim 3, wherein
each R² is the group Y—X—;
each X is independently selected from —O-Z-, —S-Z-, —SO-Z-, —SO₂-Z-, —CON(R⁶)-Z-, —SO₂N(R⁶)-Z-, and —CH═CH-Z-;
each Y is independently selected from phenyl-Z¹-, naphthyl-Z¹-, heterocyclyl-Z¹-, C₃₋₇ cycloalkyl-Z¹-, C₁₋₆ alkyl, and C₂₋₆ alkenyl; and
each Y is independently optionally substituted with R⁴.

8. A compound according to claim 3, wherein each R⁴ is independently selected from halo, —CH₃₋ₐFₐ, CN, NO₂, C₁₋₆ alkyl, —OC₁₋₆ alkyl, —COOH, —C(O)OC₁₋₆ alkyl, OH, or phenyl.

9. A compound of Formula (II) or a salt, solvate, or prodrug thereof 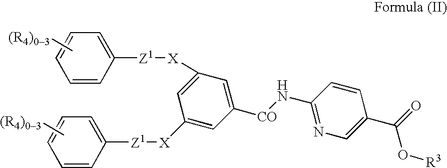
wherein
R³ is selected from hydrogen and C₁₋₆ alkyl;
each R⁴ is independently selected from halo, —CH₃₋ₐFₐ, CN, NO₂, NH₂, C₁₋₆ alkyl, —OC₁₋₆ alkyl, —COOH, —C(O)OC₁₋₆ alkyl, OH, phenyl, or R⁵—X;
R⁵ is selected from hydrogen, C₁₋₆ alkyl, —CH₃₋ₐFₐ, phenyl, naphthyl, heterocyclyl, or C₃₋₇ cycloalkyl, and is optionally substituted with halo, C₁₋₆ alkyl, —CH₃₋ₐFₐ, CN, NO₂, NH₂, COOH or —C(O)OC₁₋₆ alkyl, and each phenyl, naphthyl, or heterocyclyl ring in R⁵ is optionally substituted with halo, CH₃₋ₐFₐ CN, NO₂, NH₂, C₁₋₆ alkyl, —OC₁₋₆ alkyl, COOH, —C(O)OC₁₋₆ alkyl, or OH;
R₆ is independently selected from hydrogen, C₁₋₆ alkyl, or —C₂₋₄ alkyl-O—C₁₋₄ alkyl;
X is a linker independently selected from —O-Z-, —C(O)O-Z-, —OC(O)-Z-, —S-Z-, —SO-Z-, —SO₂-Z-, —N(R⁶)-Z-, —N(R⁶)SO₂-Z-, —SO₂N(R⁶)-Z-, —(CH₂)₁₋₄—, —CH═CH-Z-, —C≡C-Z-, —N(R⁶)CO-Z-, —CON(R⁶)-Z-, —C(O)N(R⁶)S(O)₂-Z-, —S(O)₂N(R⁶)C(O)-Z-, —C(O)-Z-, or a direct bond;
each Z is independently a direct bond or a group of the formula —(CH₂)ₚ—C(R⁶)₂—(CH₂)_{q}—;
each Z¹ is independently a direct bond or a group of the formula —(CH₂)ₚ—C(R⁶)₂—(CH₂)_{q}—.

10. A compound of Formula (IIa) or a salt, solvate, or prodrug thereof 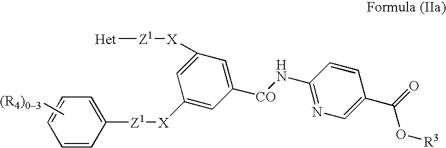
wherein
Het is a monocyclic heterocyclyl, optionally substituted with up to three groups selected from R⁴; and R³ is selected from hydrogen or C₁₋₆ alkyl;
each R⁴ is independently selected from halo, —CH₃₋ₐFₐ, CN, NO₂, NH₂, C₁₋₆ alkyl, —OC₁₋₆ alkyl, —COOH, —C(O)OC₁₋₆ alkyl, OH, phenyl, or R⁵—X;
R⁵ is selected from hydrogen, C₁₋₆ alkyl, —CH₃₋ₐFₐ, phenyl, naphthyl, heterocyclyl, or C₃₋₇ cycloalkyl, and is optionally substituted with halo, C₁₋₆ alkyl, —CH₃₋ₐFₐ, CN, NO₂, NH₂, COOH, or —C(O)OC₁₋₆ alkyl, and each phenyl, naphthyl, or heterocyclyl ring in R⁵ is optionally substituted with halo, CH₃₋ₐFₐ, CN, NO₂, NH₂, C₁₋₆alkyl, —OC₁₋₆ alkyl, COOH, —C(O)OC₁₋₆ alkyl, or OH;
R⁶ is independently selected from hydrogen, C₁₋₆ alkyl, or —C₂₋₄ alkyl-O—C₁₋₄ alkyl;
X is a linker independently selected from —O-Z-, —C(O)O-Z-, —OC(O)-Z-, —S-Z-, —SO-Z-, —SO₂-Z-, —N(R⁶)-Z-, —N(R⁶)SO₂-Z-, —SO₂N(R⁶)-Z-, —(CH₂)₁₋₄—, —CH═CH-Z-, —C≡C-Z-, —N(R⁶)CO-Z-, —CON(R⁶)-Z-, —C(O)N(R⁶)S(O)₂-Z-, —S(O)₂N(R⁶)C(O)-Z-, —C(O)-Z-, or a direct bond;
each Z is independently a direct bond or a group of the formula —(CH₂)ₚ—C(R⁶)₂—(CH₂)_{q}—;
each Z¹ is independently a direct bond or a group of the formula —(CH₂)ₚ—C(R⁶)₂—(CH₂)_{q}—.

11. A compound of Formula (Ilf) or a salt, solvate or prodrug thereof 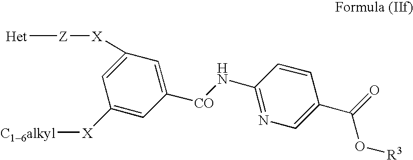
wherein
Het is a monocyclic heterocyclyl that is independently optionally substituted with up to three R⁴ groups;
C₁₋₆ alkyl is independently optionally substituted with up to three R⁴ groups;
the C₁₋₆ alkyl group optionally contains a double bond;
R³ is selected from hydrogen or C₁₋₆ alkyl;
each R⁴ is independently selected from halo, —CH₃₋ₐFₐ, CN, NO₂, NH₂, C₁₋₆ alkyl, —OC₁₋₆ alkyl, —COOH, —C(O)OC₁₋₆alkyl, OH, phenyl, or R⁵—X;
R⁵ is selected from hydrogen, C₁₋₆ alkyl, —CH₃₋ₐFₐ, phenyl, naphthyl, heterocyclyl, or C₃₋₇ cycloalkyl, and is optionally substituted with halo, C₁₋₆ alkyl, —CH₃₋ₐFₐ, CN, NO₂, NH₂, COOH, or —C(O)OC₁₋₆ alkyl, and each phenyl, naphthyl, or heterocyclyl ring in R⁵ is optionally substituted with halo, CH₃₋ₐFₐ, CN, NO₂, NH₂, C₁₋₆ alkyl, —OC₁₋₆ alkyl, COOH, —C(O)OC₁₋₆ alkyl, or OH;
X is a linker independently selected from —O-Z-, —C(O)O-Z-, —OC(O)-Z-, —S-Z-, —SO-Z-, —SO₂-Z-, —N(R⁶)-Z-, —N(R⁶)SO₂-Z-, —SO₂N(R⁶)-Z-, —(CH₂)₁₋₄—, —CH═CH-Z-, —C≡C-Z-, —N(R⁶)CO-Z-, —CON(R⁶)-Z-, —C(O)N(R⁶)S(O)₂-Z-, —S(O)₂N(R⁶)C(O)-Z-, —C(O)-Z-, or a direct bond; and
each Z is independently a direct bond or a group of the formula —(CH₂)ₚ—C(R⁶)₂—(CH₂)_{q}—.

12. A compound according to any one of claims 9 to 11, wherein
X is independently selected from: —O-Z-, SO₂N(R⁶)-Z-, or —N(R⁶)-Z-;
Z is a direct bond or —CH₂—;
Z¹ is selected from a direct bond, —CH₂——(CH₂)₂—, or 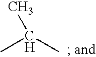
R³ is selected from hydrogen or C₁₋₆ alkyl;or a salt, solvate or prodrug thereof.

13. A pharmaceutical composition comprising a compound according to any one of claims 3, 9, 10, or 11 or a salt, solvate or prodrug thereof, together with a pharmaceutically acceptable diluent or carrier.

14. A method for the treatment or prevention of a disease or medical condition mediated through GLK, comprising administering a compound of claim 1 or a salt, prodrug, or solvate thereof, with the proviso that when R³ is hydrogen or methyl, m is 2, and n is 0, then (R¹)ₘ is other than di-C₁₋₄ alkyl.

15. A process for the preparation of a compound of Formula (I) 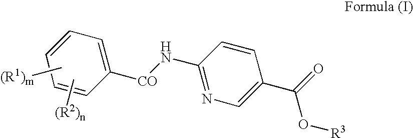
wherein
each R¹ is independently selected from OH, —(CH₂)₁₋₄OH, —CH₃₋ₐFₐ, —(CH₂)₁₋₄CH₃₋ₐFₐ, halo, C₁₋₆alkyl, C₂₋₆alkenyl, C₂₋₆alkynyl, NO₂, NH₂, —NH—C₁₋₄ alkyl, —N-di-(C₁₋₄ alkyl), CN, or formyl;
each R² is the group Y—X—;
R³ is selected from hydrogen or C₁₋₆ alkyl;
each R⁴ is independently selected from halo, —CH₃₋ₐFₐ, CN, NO₂, NH₂, C₁₋₆ alkyl, —OC₁₋₆ alkyl, —COOH, —C(O)OC₁₋₆ alkyl, OH, phenyl, or R⁵—X;
R⁵ is selected from hydrogen, C₁₋₆ alkyl, —CH₃₋ₐFₐ, phenyl, naphthyl, heterocyclyl, or C₃₋₇ cycloalkyl, and is optionally substituted with halo, C₁₋₆ alkyl, —CH₃₋ₐFₐ, CN, NO₂, NH₂, COOH, or —C(O)OC₁₋₆ alkyl, and each phenyl, naphthyl, or heterocyclyl ring in R⁵ is optionally substituted with halo, CH₃₋ₐFₐ, CN, NO₂, NH₂, C₁₋₆ alkyl, —OC₁₋₆ alkyl, COOH, —C(O)OC₁₋₆ alkyl, or OH;
R⁶ is independently selected from hydrogen, C₁₋₆ alkyl, or —C₂₋₄ alkyl-O—C₁₋₄ alkyl;
each X is a linker independently selected from —O-Z-, —O-Z-O-Z-, —C(O)O-Z-, —OC(O)-Z-, —S-Z-, —SO-Z-, —SO₂-Z-, —N(R⁶)-Z-, —N(R⁶)SO₂-Z-, —SO₂N(R⁶)-Z-, —(CH₂)₁₋₄—, —CH═CH-Z-, —C≡C-Z-, —N(R⁶)CO-Z-, —CON(R⁶)-Z-, —C(O)N(R⁶)S(O)₂-Z-, —S(O)₂N(R⁶)C(O)-Z-, —C(O)-Z-, or a direct bond;
each Y is independently selected from aryl-Z¹-, heterocyclyl-Z¹-, C₃₋₇cycloalkyl-Z¹-, C₁₋₆ alkyl, C₂₋₆alkenyl, C₂₋₆ alkynyl, or —(CH₂)₁₋₄CH₃₋ₐFₐ, wherein Y is independently optionally substituted with up to three R⁴ groups;
each Z is independently a direct bond or a group of the formula —(CH₂)ₚ—C(R⁶)₂—(CH₂)_{q}—;
each Z¹ is independently a direct bond or a group of the formula —(CH₂)ₚ—C(R⁶)₂—(CH₂)_{q}—;
each a is independently 1, 2, or 3;
m is 0, 1, or 2;
n is 0, 1, 2, 3, or 4;
and n+m>0;
p is an integer between 0 and 2;
q is an integer between 0 and 2; and
p+q<4;
which comprises
(a) reaction of a compound of Formula (IIIa) with a compound of Formula (IIIb), 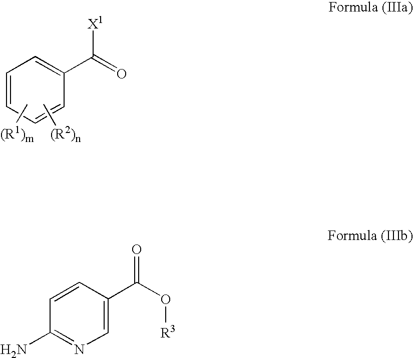
or, wherein X¹ is a leaving group
(b) for compounds of Formula (I), wherein R³ is hydrogen, deprotection of a compound of Formula (IIIc), 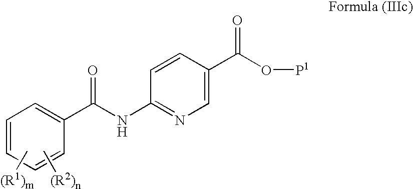
wherein P¹ is a protecting group;
(c) for compounds of Formula (I), wherein n is 1, 2, 3, or 4, reaction of a compound of Formula (IIId) with a compound of Formula (IIIe),
Y—X″
Formula (IIId) 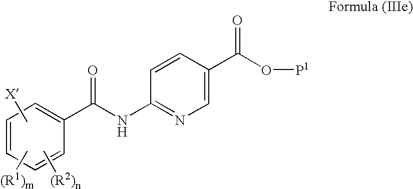
wherein X′ and X″ comprise groups which, when reacted together form the group X;
(d) for a compound of Formula (I), wherein n is 1, 2, 3 or 4 and X or X¹ is —SO-Z- or —SO₂-Z-, oxidation of the corresponding compound of Formula (I) wherein X or X¹ respectively is —S-Z-;
(e) reaction of a compound of Formula (IIIf) with a compound of Formula (IIIg), 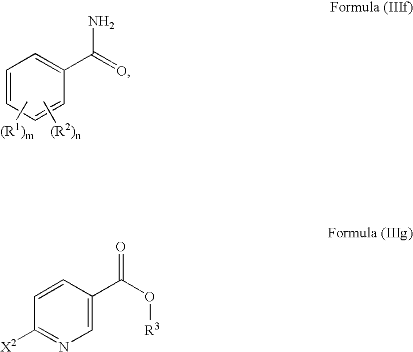
wherein X² is a leaving group;
and thereafter, if necessary:
i) converting a compound of Formula (I) into another compound of Formula (I);
ii) removing any protecting groups;
iii) forming a salt, prodrug or solvate thereof.
